(19) United States
(12) Patent Application Publication
(10) Pub. No.: US 2019/0099209 A1
(43) Pub. Date: Apr. 4, 2019

(54) BIPOLAR ELECTRODE SALINE LINKED CLOSED LOOP MODULATED VACUUM SYSTEM

(71) Applicant: Ethicon LLC, Guaynabo, PR (US)

(72) Inventors: David A. Witt, Maineville, OH (US); David C. Yates, West Chester, OH (US); Jeffrey D. Messerly, Cincinnati, OH (US); Meghan R. Tighe, Cypress, TX (US); Monica L. Rivard, Cincinnati, OH (US); Chad P. Boudreaux, Cincinnati, OH (US); Cory G. Kimball, Hamilton, OH (US); Frederick E. Shelton, IV, Hillsboro, OH (US)

(21) Appl. No.: 15/720,810

(22) Filed: Sep. 29, 2017

(51) Int. Cl. A61B 18/08

(52) U.S. Cl. CPC A61B 18/082; A61B 2018/00595

(57) ABSTRACT

An end effector of an electrosurgical device may include a fluid discharge port, a fluid aspiration port, and at least two electrodes, in which the electrodes are disposed on a surface of a body of the end effector. The end effector body may include channels fluidically coupled to the fluid discharge port. The end effector body may also include channels to receive the electrodes. The electrodes may be helically wound about the end effector body. The electrodes may interdigitate. An electrosurgical device may include the end effector which is fluidically, mechanically, and electrically coupled to a handle assembly by a shaft assembly. The shaft assembly may be bendable and assume a bent configuration upon the application of a force orthogonal to a longitudinal axis of the shaft assembly. The shaft assembly may retain the bent configuration until the application of a countering force.

### BACKGROUND

Many internal surgical procedures require the removal of tissue as part of the surgical procedure. The removal of such tissue invariably results in severing multiple blood vessels leading to localized blood loss. Significant blood loss may comprise the patient's health by potentially leading to hypovolemic shock. Even minor blood loss may complicate the surgery by resulting in blood pooling into the surgical site, thereby obscuring the visibility of the tissue from the surgeons and surgical assistants. The problem of blood loss into the surgical site may be especially important in broad area surgeries, such as liver resection, in which multiple blood vessels may be severed during the procedure.

Typically, an electrosurgical cautery device is used to seal the blood vessels, thereby preventing blood loss. Such electrosurgical cautery devices may include bipolar devices that incorporate a pair of electrodes that are powered by RF (radiofrequency) energy to heat and cauterize the tissue and blood vessels. Direct application of the electrodes to the tissue may lead to unwanted effects such as localized tissue charring and fouling of the electrodes by charred tissue matter sticking to them.

A method to reduce charring and fouling may include introducing a saline fluid into the surgical site to irrigate the site. Alternatively, the saline fluid may be heated by the electrodes to form a steam to cauterize the tissue. In this manner, the tissue is not placed in direct contact with the electrodes and electrode fouling is prevented. Although a saline fluid may be used, any electrically conducting fluid (for example, an aqueous mixture containing ionic salts) may be used to promote steam-based cauterization. After the steam cauterizes the tissue by transferring its heat thereto, the steam may condense to water. The resulting water may be used to clear the surgical site of unwanted material such as the remnants of the cauterized tissue. An aspirator may be used to remove the mixture of water and tissue remnants. It may be difficult and inefficient for the surgeon to cauterize and aspirate the tissue especially if separate devices are required. Thus, a device incorporating the cauterization and aspiration functions is desirable.

The incorporation of both a saline source and an evacuation source for aspiration into a bipolar electrosurgical cautery instrument may be problematic. If the aspirator operates continuously, then the saline may not reside in contact with the electrodes long enough to be heated and form steam. If the saline source operates continuously, then excess saline may be delivered to the surgical site and obscure the area from the surgeon. It is possible to have a device with multiple actuators to allow the surgeon to selectively emit a fluid to be vaporized by the electrodes and evacuate the surgical site. However, such multiple actuators may be clumsy to use and lead to hand and finger fatigue during a long surgical procedure.

Therefore, it is desirable to have a device that permits a surgeon to effectively and efficiently provide steam cauterization and tissue mixture aspiration to a surgical site without requiring excessive manipulation of the surgical device.

### SUMMARY

In one aspect, an end effector of an electrosurgical device may include an end effector body having a longitudinal body axis, at least one distal fluid discharge port disposed in the end effector body, at least one distal fluid aspiration port disposed in the end effector body, a first electrode disposed on a first portion of a surface of the end effector body, and a second electrode disposed on a second portion of the surface of the end effector body, in which the at least one distal fluid discharge port is configured to discharge a fluid therefrom and the end effector body is configured to direct the discharged fluid to contact a surface of the first electrode and a surface of the second electrode.

In one aspect of the end effector, the first electrode includes at least one first electrode component disposed parallel to the longitudinal body axis and the second electrode has at least one second electrode component disposed parallel to the longitudinal body axis.

In an aspect of the end effector, the first electrode includes at least one first electrode component disposed helically along the longitudinal body axis and the second electrode has at least one second electrode component disposed helically along the longitudinal body axis.

In an aspect of the end effector, the first electrode has at least one circular first electrode component disposed orthogonal to the longitudinal body axis and the second electrode has at least one circular second electrode component disposed orthogonal to the longitudinal body axis.

In an aspect of the end effector, the end effector body has one or more body features.

In an aspect of the end effector, the one or more body features are configured to direct a flow of the discharged fluid about the surface of the end effector body.

In an aspect of the end effector, the body features have one or more protruding features from the surface of the end effector body.

In an aspect of the end effector, the one or more protruding features have one more protruding features helically disposed about the end effector body and oriented along the longitudinal body axis.

In an aspect of the end effector, the one or more protruding features have one or more raised rings from the surface of the end effector body.

In an aspect of the end effector, at least a portion of the first electrode is disposed on a surface of the one or more protruding features.

In an aspect of the end effector, at least a portion of the second electrode is disposed on a surface of the one or more protruding features.

In an aspect of the end effector, the body features have one or more channels in the surface of the end effector body.

In an aspect of the end effector, the one or more channels have one more channels helically disposed within the surface of the end effector body and oriented along the longitudinal body axis.

In an aspect of the end effector, the one or more channels include one or more channels disposed parallel to the longitudinal body axis of the end effector body.

In an aspect of the end effector, at least a portion of the first electrode is disposed within the one or more channels.

In an aspect of the end effector, at least a portion of the second electrode is disposed within the one or more channels.

In an aspect of the end effector, the at least one distal fluid aspiration port is disposed at a distal end of the end effector body.

In an aspect of the end effector, at least a portion of the end effector body is tapered towards a distal end of the end effector body.

In one aspect, an electrosurgical device includes an end effector having an end effector body, a first electrode disposed on a first portion of a surface of the end effector body, a second electrode disposed on a second portion of the surface of the end effector body, at least one fluid discharge port disposed on a surface of the end effector body, and at least one fluid aspiration port disposed at a distal end of the end effector body. The electrosurgical device may also include a shaft having a longitudinal shaft axis, in which a distal shaft end is in mechanical communication with a proximal end of the end effector body. The electrosurgical device may further include a housing having a longitudinal housing axis, the housing further including a fluid source port configured to receive a first fluid from a first fluid source and fluidically coupled to the at least one distal fluid discharge port, a fluid evacuation port configured to deliver a second fluid to a vacuum source and fluidically coupled to the at least one distal fluid aspiration port, a first fluid control fluidically coupled to the at least one fluid discharge port, and a combination control, configured to regulate a flow of the first fluid to the first fluid control and to regulate an amount of power delivered to the first electrode and the second electrode from a power source.

In an aspect of the electrosurgical device, a distal end of the housing is in mechanical communication with a proximal end of the shaft end configured so that the longitudinal housing axis forms an acute angle with respect to the longitudinal shaft axis.

In an aspect, the electrosurgical device may further include an end effector unit composed of the end effector body and an end effector body extension mechanically coupled to a proximal portion of the end effector body.

In an aspect of the electrosurgical device, the end effector unit is disposed within an interior space of the shaft and at least a portion of an evacuation tube is disposed within an interior space of the end effector unit.

In an aspect of the electrosurgical device, the end effector unit has one or more fluid vents disposed proximate to an outer surface of the at least portion of the evacuation tube thereby creating a fluid space defined by the outer surface of the evacuation tube and an inner surface of the end effector body extension.

In an aspect of the electrosurgical device, the fluid space is fluidically coupled to the at least one fluid discharge port.

In an aspect of the electrosurgical device, the evacuation tube is fludically coupled to the fluid evacuation port.

In an aspect of the electrosurgical device, the evacuation tube is electrically conducting and the first electrode is electrically coupled to the evacuation tube.

In an aspect of the electrosurgical device, the end effector body has one or more channels configured to receive the first electrode and the second electrode.

In one aspect, an electrosurgical device includes an end effector having an end effector body, a first electrode disposed on a first portion of a surface of the end effector body, a second electrode disposed on a second portion of the surface of the end effector body, at least one fluid discharge port disposed at a distal end of the end effector body, and at least one fluid aspiration port disposed at the distal end of the end effector body. The electrosurgical device may further include a shaft having a longitudinal shaft axis, wherein a distal shaft end is in mechanical communication with a proximal end of the end effector body, and wherein the shaft is configured to assume a bent configuration upon receiving an application of a first force orthogonal to a longitudinal axis of the shaft. The electrosurgical device may further include a housing having a fluid source port configured to receive a first fluid from a first fluid source and fluidically coupled to the at least one distal fluid discharge port, and a fluid evacuation port configured to deliver a second fluid to a vacuum source and fluidically coupled to the at least one distal fluid aspiration port.

In an aspect of the electrosurgical device, the shaft is configured to remain in the bent configuration after the removal of the first force applied to the shaft.

In an aspect of the electrosurgical device, the shaft is configured to assume an unbent configuration and upon receiving an application of a second force to the shaft, wherein the second force is an opposing force to the first force.

In an aspect of the electrosurgical device, the first electrode disposed on the first portion of the surface of the end effector body is helically wound about a longitudinal axis of the end effector body, and the second electrode disposed on the second portion of the surface of the end effector body is helically wound about the longitudinal axis of the end effector body.

In an aspect of the electrosurgical device, the first electrode disposed on the first portion of the surface of the end effector body has a first plurality of legs disposed on the surface of the end effector body and parallel to a longitudinal axis of the end effector body, and the second electrode disposed on the second portion of the surface of the end effector body has a second plurality of legs disposed on the surface of the end effector body and parallel to the longitudinal axis of the end effector body.

### BRIEF DESCRIPTION OF THE FIGURES

The features of the various aspects are set forth with particularity in the appended claims. The various aspects, however, both as to organization and methods of operation, together with advantages thereof, may best be understood by reference to the following description, taken in conjunction with the accompanying drawings as follows:

FIG. 1 illustrates a perspective view of one aspect of an electrosurgical device.

FIG. 2 illustrates an expanded view of one aspect of an end effector of the electrosurgical device depicted in FIG. 1.

FIG. 3 illustrates a side perspective view of one aspect of the electrosurgical device depicted in FIG. 1.

FIGS. 4, 5, and 6 illustrate plan views of the bottom, side, and top, respectively, of one aspect of the electrosurgical device depicted in FIG. 1.

FIG. 7 illustrates a partial sectional perspective view of one aspect of the electrosurgical device depicted in FIG. 1.

FIG. 8 illustrates a side perspective view of a surgical device with spreadable bipolar electrodes, according to some aspects of the present disclosure.

FIG. 9 illustrates a side plan view and a top plan view of the surgical device depicted in FIG. 8 along with two perspective views of a distal end of the surgical device of FIG. 8, according to some aspects of the present disclosure.

FIGS. 10A and 10B illustrate a perspective view of a distal end of the surgical device of FIG. 8, depicting the spreadable bipolar electrodes in a closed state and in an open state, respectively, according to some aspects of the present disclosure.

FIG. 11 depicts one example control mechanism of an surgical device having spreadable electrodes as depicted in FIGS. 8-10, according to some aspects of the present disclosure.

FIG. 12 depicts an exterior view of another aspect of an electrosurgical device according to some aspects of the present disclosure.

FIG. 13 depicts an exterior view of a Yankauer suction device.

FIGS. 14A,B depict interior views of the electrosurgical device depicted in FIG. 12 according to some aspects of the present disclosure.

FIG. 15A depicts another interior views of the electrosurgical device depicted in FIG. 12 according to some aspects of the present disclosure.

FIGS. 15B,C depict alternative aspects of an end effector of the electrosurgical device depicted in FIG. 12 according to some aspects of the present disclosure.

FIGS. 16A,B depict expanded views of aspects of device control components of the electrosurgical device depicted in FIG. 12 according to some aspects of the present disclosure.

FIG. 17 is a sectional perspective view of a distal end of the electrosurgical device depicted in FIG. 12 illustrating a fluid flow therethrough, according to some aspects of the present disclosure.

FIG. 18 is a sectional perspective view of a proximal end of the electrosurgical device depicted in FIG. 12 illustrating a fluid flow therethrough, according to some aspects of the present disclosure.

FIG. 19 depicts a further perspective view of a proximal end of the electrosurgical device depicted in FIG. 12 illustrating a fluid flow therethrough, according to some aspects of the present disclosure.

FIG. 20A depicts an exploded view of handle components of the electrosurgical device depicted in FIG. 12 according to some aspects of the present disclosure.

FIG. 20B depicts an exploded view of shaft and end effector components of the electrosurgical device depicted in FIG. 12 according to some aspects of the present disclosure.

FIGS. 21A-F depict, in perspective view, sequential assembly drawings of one aspect of shaft and end effector components of the electrosurgical device depicted in FIG. 20B according to some aspects of the present disclosure.

FIG. 22A depicts, in a side perspective view, an assembly drawing of a second aspect of shaft and end effector components of an electrosurgical device having an end effector as depicted in FIG. 15C, according to some aspects of the present disclosure.

FIG. 22B depicts a perspective view of an assembled end effector as depicted in FIG. 15C, according to some aspects of the present disclosure.

FIG. 22C depicts a side perspective view of an assembled shaft assembly of a electrosurgical device having an end effector as depicted in FIG. 15C, according to some aspects of the present disclosure.

FIGS. 23A-C depict, in perspective view, sequential assembly drawings of an aspect of fluid flow components of the electrosurgical device depicted in FIG. 20A according to some aspects of the present disclosure.

FIG. 23D depicts, in partial cross-sectional perspective view, the assembled fluid flow components of FIGS. 23A-C disposed within a handle portion of the electrosurgical device depicted in FIG. 20A according to some aspects of the present disclosure.

FIGS. 24A-C depict, in perspective view, sequential assembly drawings of an aspect of fluid control components of the electrosurgical device depicted in FIG. 20A according to some aspects of the present disclosure.

FIGS. 25A-C depict, in perspective view, sequential assembly drawings of an aspect of electrical components of the electrosurgical device depicted in FIG. 20A according to some aspects of the present disclosure.

FIG. 25D depicts, in perspective view, the assembled electrical components depicted in FIGS. 25A-C as disposed within the assembled electrosurgical device depicted in FIG. 20A, wherein a top portion of a handle assembly of the electrosurgical device is shown in transparent view, according to some aspects of the present disclosure.

FIG. 26A depicts a side perspective view of another aspect of an electrosurgical device that includes an end effector having helically wound electrodes according to some aspects of the present disclosure.

FIG. 26B depicts an expanded perspective view of the end effector of the electrosurgical device depicted in FIG. 26A, according to some aspects of the present disclosure.

FIG. 27A depicts a side plan view of the electrosurgical device depicted in FIG. 26A, according to some aspects of the present disclosure.

FIGS. 27B,C depict side sectional views of the electrosurgical device depicted in FIG. 26A, according to some aspects of the present disclosure.

FIG. 27D depicts a top perspective view of the electrosurgical device depicted in FIG. 26A, according to some aspects of the present disclosure.

FIG. 27E depicts a top perspective sectional view of the electrosurgical device depicted in FIG. 27D, according to some aspects of the present disclosure.

FIGS. 28A,B depict a side sectional view of a proximal end of the electrosurgical device depicted in FIG. 27D, according to some aspects of the present disclosure.

FIG. 29A depicts an expanded view of the end effector of the electrosurgical device depicted in FIG. 26A according to some aspects of the present disclosure.

FIGS. 29B,C depict side cross sectional views of the end effector depicted in FIG. 29A according to some aspects of the present disclosure.

FIG. 30A depicts a side plan view of another aspect of an end effector having helically wound electrodes according to some aspects of the present disclosure.

FIG. 30B depicts a distal end plan view of the end effector depicted in FIG. 30A, according to some aspects of the present disclosure.

FIG. 31 depicts an expanded view of an end effector having helically wound electrodes according to some aspects of the present disclosure.

FIG. 32A depicts another aspect of an electrosurgical device that includes an end effector having interdigitated electrodes according to some aspects of the present disclosure.

FIG. 32B depicts an expanded perspective view of the end effector of the electrosurgical device depicted in FIG. 32A, according to some aspects of the present disclosure.

FIGS. 32C,D depict a distal and a proximal perspective view, respectively, of the electrosurgical device depicted in FIG. 32A, according to some aspects of the present disclosure.

FIG. 32E depicts a side sectional view of the electrosurgical device depicted in FIG. 32A, according to some aspects of the present disclosure.

FIGS. 32F,G depict side sectional views of the end effector of the electrosurgical device depicted in FIG. 32A, according to some aspects of the present disclosure.

FIG. 33 depicts an expanded view of the end effector of the electrosurgical device depicted in FIG. 32A according to some aspects of the present disclosure.

FIG. 34 depicts an expanded view of an end effector having interdigitated electrodes according to some aspects of the present disclosure.

FIG. 35 depicts another aspect of an end effector, according to some aspects of the present disclosure.

FIG. 36 depicts another aspect of an electrosurgical device, according to some aspects of the present disclosure.

FIG. 37 depicts a top plan view and a side plan view of an aspect of an electrosurgical device having a flexible shaft according to some aspects of the present disclosure.

FIG. 38 depicts an aspect of a handle assembly for a modular electrosurgical device according to some aspects of the present disclosure.

FIG. 39A depicts a top perspective view of a modular electrosurgical device according to some aspects of the present disclosure.

FIGS. 39B,C depict a side and a top plan view, respectively, of the modular electrosurgical device depicted in FIG. 39A, according to some aspects of the present disclosure.

FIG.39D depicts an exploded perspective view of the modular electrosurgical device depicted in FIG. 39A, according to some aspects of the present disclosure.

FIG. 39E depicts a top and a side plan view of the modular electrosurgical device depicted in FIG. 39A having a first type of an end effector, according to some aspects of the present disclosure.

FIG. 39F depicts a top and a side perspective view of the modular electrosurgical device depicted in FIG. 39A lacking a modular end effector, according to some aspects of the present disclosure.

FIGS. 40A,B depict assembly drawings of portions of the modular electrosurgical device depicted in FIGS. 39A-C according to some aspects of the present disclosure.

FIG. 40C depicts a bottom plan view of the modular electrosurgical device depicted in FIGS. 39A-C according to some aspects of the present disclosure.

FIGS. 41A,B depict side exploded perspective views of a disassembled modular electrosurgical device as depicted in FIGS. 39A-C according to some aspects of the present disclosure.

FIG. 41C depicts sequential assembly drawings, in side perspective view, of the modular electrosurgical device as depicted in FIG. 38 according to some aspects of the present disclosure.

FIG. 42 depicts a user holding the modular electrosurgical device depicted in FIGS. 39A-C according to some aspects of the present disclosure.

FIGS. 43A-C depict perspective views of aspects of an end effector interface for the modular electrosurgical device depicted in FIGS. 39A-C according to some aspects of the present disclosure.

FIG. 44 depicts an end plan view of an aspect of an end effector interface for the modular electrosurgical device depicted in FIG. 38 according to some aspects of the present disclosure.

FIG. 45 depicts end plan views of end effector interfaces for a variety of modular end effectors configured for use with the modular electrosurgical device depicted in FIG. 38 according to some aspects of the present disclosure.

FIGS. 46 and 47 depict plan views of aspects of a variety of modular end effectors configured for use with the modular electrosurgical device depicted in FIG. 38 according to some aspects of the present disclosure.

FIGS. 48A,B depict distal and proximal perspective views, respectively, of the variety of modular end effectors configured for use with the modular electrosurgical device as depicted in FIGS. 46 and 47 according to some aspects of the present disclosure.

FIG. 49 depicts a distal perspective view of modular end effector (d) as depicted in FIG. 48A according to some aspects of the present disclosure.

FIG. 50 depicts a distal perspective view of modular end effector (c) as depicted in FIG. 48A according to some aspects of the present disclosure.

FIGS. 51A,B,C depict top plan views of modular end effectors (a), (b), and (c), respectively, as depicted in FIG. 48A according to some aspects of the present disclosure.

FIG. 52 depicts a top plan view of modular end effector (d) as depicted in FIG. 48A according to some aspects of the present disclosure.

FIG. 53 depict distal perspective views of modular end effectors (a)-(d) as depicted in FIG. 48A according to some aspects of the present disclosure.

FIGS. 54A,B depict a top plan view and a side perspective view, respectively, of an aspect of a conical modular end effector according to some aspects of the present disclosure.

FIGS. 55A,B depict a distal end plan view and a bottom perspective view, respectively, of a three-face modular end effector according to some aspects of the present disclosure.

FIGS. 56A,B depict a top plan view and a side perspective view, respectively, of a second aspect of a conical modular end effector according to some aspects of the present disclosure.

FIGS. 57A,B,C,D depict, respectively, a distal end perspective view, a top plan view, a side plan view, and a side cross-sectional view, of representative modular end effectors (a), (b), and (c) as depicted in FIG. 48A according to some aspects of the present disclosure.

FIG. 58 depicts, a distal end perspective view of modular end effector (f) as depicted in FIG. 48A according to some aspects of the present disclosure.

FIGS. 59A-C depict various perspective views of modular end effector (f) as depicted in FIG. 48A according to some aspects of the present disclosure.

FIGS. 59D-F depict various plan views of modular end effector (f) as depicted in FIG. 48A according to some aspects of the present disclosure.

FIG. 60 depicts a distal end perspective view of modular end effector (e) as depicted in FIG. 48A according to some aspects of the present disclosure.

FIGS. 61A and B depict a bottom plan view and a side plan view, respectively, of modular end effector (e) as depicted in FIG. 48A according to some aspects of the present disclosure.

FIGS. 61C and D depict a bottom perspective view and a distal end perspective view, respectively, of modular end effector (e) as depicted in FIG. 48A according to some aspects of the present disclosure.

FIG. 62A depicts a perspective view of an aspect of an end effector having recessed electrodes according to some aspects of the present disclosure.

FIG. 62B depicts a cross-sectional view of the aspect of the end effector depicted in FIG. 62A according to some aspects of the present disclosure.

FIG. 63A schematically depicts a tissue contacted by an end effector of an electrosurgical device, in which the electrodes directly contact the tissue according to some aspects of the present disclosure.

FIG. 63B schematically depicts a tissue contacted by an end effector of an electrosurgical device, in which the electrodes are recessed below the surface of the end effector according to some aspects of the present disclosure.

### DETAILED DESCRIPTION

Applicant of the present application owns the following patent applications filed concurrently herewith and which are each herein incorporated by reference in their respective entireties:

Attorney Docket No. END8257USNP/170168, titled IMPROVING SALINE CONTACT WITH ELECTRODES, by inventors Mark A. Davison et al., filed on even date herewith.

Attorney Docket No. END8258USNP/170169, titled SYSTEMS AND METHODS FOR MANAGING FLUID AND SUCTION IN ELECTROSURGICAL SYSTEMS, by inventors David A. Witt et al., filed on even date herewith.

Attorney Docket No. END8259USNP/170170, titled FLEXIBLE ELECTROSURGICAL INSTRUMENT, by inventors David A. Witt et al., filed on even date herewith.

As disclosed above, an electrosurgical device may incorporate functions to cauterize and aspirate tissues during a broad area surgical procedure. In some electrosurgical devices, energized electrodes may be used to perform the cauterization procedure. However, as also disclosed above, the electrodes of such devices may be susceptible to fouling by the tissue contacted by the electrodes during cauterization. It may be appreciated that cauterization of tissue may be accomplished by exposing the tissue to a heated material other than the electrodes. As also disclosed above, in one non-limiting example, a fluid, such as a saline fluid, may be heated by the electrodes and the heated fluid or steam may then be used to cauterize the tissue. The saline, or other conductive fluid, may be heated by an electrical current flowing between the electrodes. In this manner, the temperature used to cauterize the tissue may be limited by the temperature of the steam (for example, at around 100° C.) thereby reducing the potential of tissue charring. Further, the surrounding tissue may be moistened by the steam, thereby preventing desiccation due to their proximity to a heated device. Additionally, the steam, upon losing heat by contacting the tissue, may condense to water, and the water may then be used to irrigate the surgical site. In this manner, a saline fluid may be used for the dual purposes of cauterization and irrigation, thereby increasing the efficiency of the cauterization procedure.

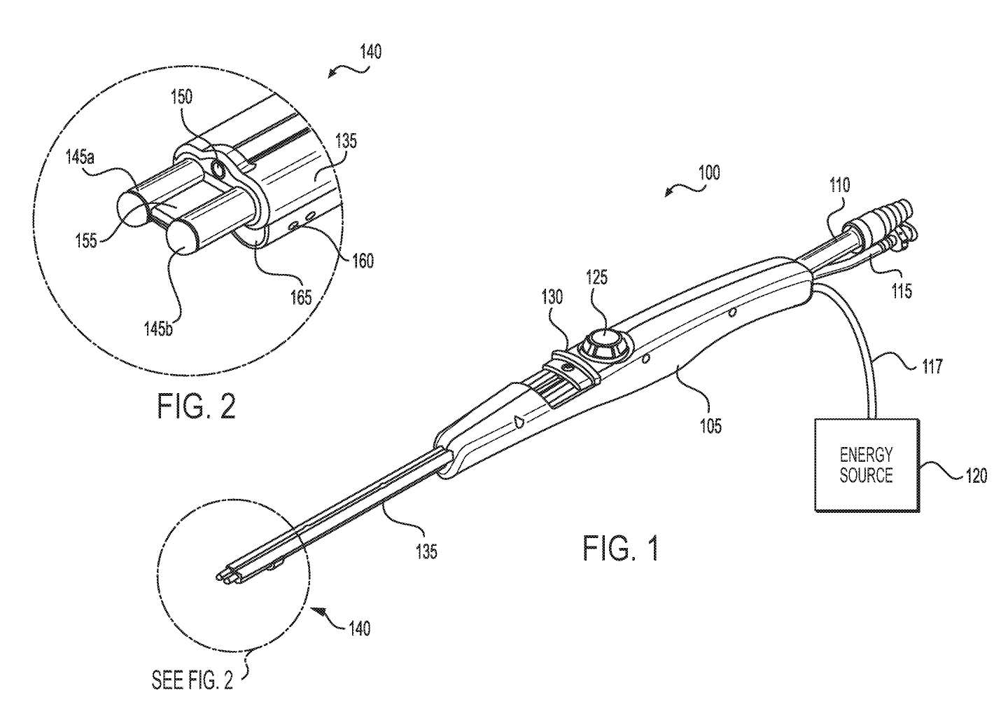

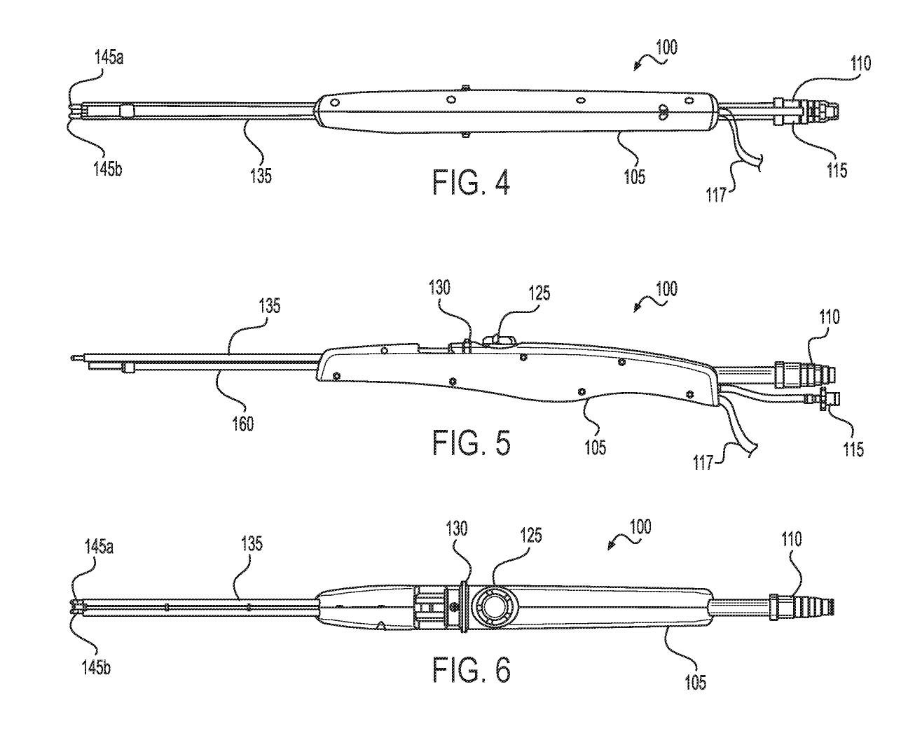

FIGS. 1-7 depict views of one example of such an electrosurgical device 100. For FIGS. 1-7, common reference numbers refer to common components within the figures.

The electrosurgical device 100 may include a housing 105 with a shaft 135 extending distally from the housing 105. The housing 105 may include, on a proximal end, a proximal fluid source port 115 and a proximal fluid evacuation port 110. In some electrosurgical device systems, the proximal fluid source port 115 may be placed in fluid communication with a source of a fluid, for example saline, buffered saline, Ringer's solution, or other electrically conducting fluids such as aqueous fluids containing ionic salts. The fluid source may operate as a gravity feed source or it may include components to actively pump the fluid into the proximal fluid source port 115. An actively pumping fluid source may include, without limitation, a power supply, a pump, a fluid source, and control electronics to allow a user to actively control the pumping operation of the actively pumping fluid source. In some electrosurgical device systems, the fluid evacuation port 110 may be placed in fluid communication with a vacuum source. The vacuum source may include a power supply, a pump, a storage component to store material removed by the vacuum source, and control electronics to allow a user to actively control the pumping operation of the vacuum source.

In addition, the housing 105 may include a connector 116 to which a cable 117 of an energy source 120 may be attached. The energy source 120 may be configured to supply energy (for example RF or radiofrequency energy) to the electrodes 145a,b. The energy source 120 may include a generator configured to supply power to the electrosurgical device 100 through external means, such as through the cable 117. In certain instances, the energy source 120 may include a microcontroller coupled to an external wired generator. The external generator may be powered by AC mains. The electrical and electronic circuit elements associated with the energy source 120 may be supported by a control circuit board assembly, for example. The microcontroller may generally comprise a memory and a microprocessor (“processor”) operationally coupled to the memory. The electronic portion of the energy source 120 may be configured to control transmission of energy to electrodes 145a,b at the end effector 140 of the electrosurgical device 100. It should be understood that the term processor as used herein includes any suitable microprocessor, microcontroller, or other basic computing device that incorporates the functions of a computer's central processing unit (CPU) on an integrated circuit or at most a few integrated circuits. The processor may be a multipurpose, programmable device that accepts digital data as input, processes it according to instructions stored in its memory, and provides results as output. It is an example of sequential digital logic, as it has internal memory. Processors operate on numbers and symbols represented in the binary numeral system. The energy source 120 may also include input devices to allow a user to program the operation of the energy source 120.

The housing 105 may also include one or more activation devices to permit a user to control the functions of the electrosurgical device 100. In some non-limiting example, the electrosurgical device 100 may include a metering valve 125 that may be activated by a user to control an amount of fluid flowing through the electrosurgical device and provide, at the distal end, an amount of the fluid to the end effector 140. In some non-limiting examples, the metering valve 125 may also permit the user to control an amount of energy supplied by the energy source 120 to the electrodes 145a,b at the end effector 140. As an example, the metering valve 125 may comprise a screw activation pinch valve to regulate the flow of fluid through the electrosurgical device 100. Additionally, the metering valve 125 may have a push-button activation function to permit current to flow from the energy source 120 to the electrodes 145a,b upon depression of the push-button by a user. It may be recognized that in some non-limiting examples, the housing 105 may include a metering valve 125 to allow regulation of fluid flow through the electrosurgical device 100 and a separate energy control device to control the amount of current sourced to the electrodes 145a,b.

The housing 105 may also be attached to a shaft 135 at a distal end of the housing 105. An end effector 140 may be associated with a distal end of the shaft 135. The end effector 140 may include electrodes 145a,b that may be in electrical communication with the energy source 120 and may receive electrical power therefrom. In some non-limiting examples, a first electrode 145a may receive electrical energy of a first polarity (such as a positive polarity) from the energy supply 120 and the second electrode 145b may receive electrical energy of a second and opposing polarity (such as a negative polarity) from the energy supply 120. Alternatively, the first electrode 145a may be connected to a ground terminal of the energy supply 120, and the second electrode 145b may be connected to a varying AC voltage terminal of the energy supply 120. The electrodes 145a,b may extend beyond the distal end of the shaft 135. The extended ends of the electrodes 145a,b be separated by a diverter 155. The diverter 155 may contact the first electrode 145a at a first edge of the diverter 155, and the diverter 155 may contact the second electrode 145b at a second edge of the diverter 155. The diverter 155 may comprise an electrically insulating material and/or a heat resistant material, which may include, without limitation a plastic such as a polycarbonate or a ceramic. The diverter 155 may be deformable or non-deformable. In some non-limiting examples, the housing 105 may include a mechanism to control a shape of a deformable diverter 155.

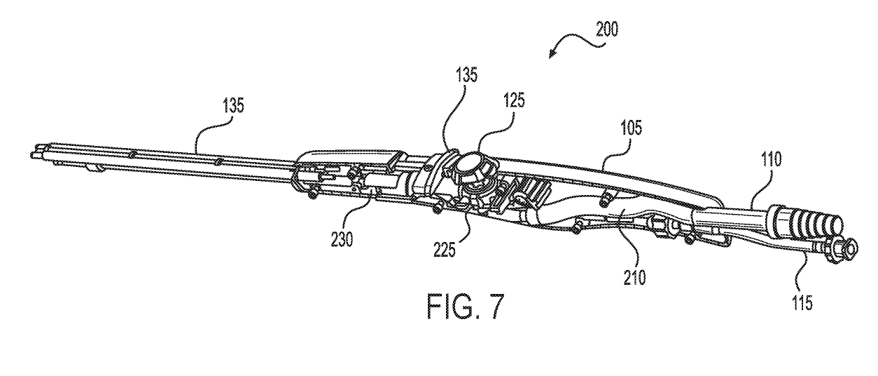

The end effector 140 may also include a fluid discharge port 150 that may be in fluid communication with the fluid source port 115 through a first fluid path. The first fluid path, such as a source fluid path, may permit the fluid to flow from the fluid source port 115 to the fluid discharge port 150. In some non-limiting examples, the fluid discharge port 150 may be positioned above the diverter 155 so that a fluid emitted by the fluid discharge port 150 may be collected on a top surface of the diverter 155. The end effector may also include a fluid aspiration port 165 that may be in fluid communication with the fluid evacuation port 110 through a second fluid path. The second fluid path, such as an aspirated fluid path (see 210 in FIG. 7), may permit a liquid mixture generated at the surgical site to flow from the fluid aspiration port 165 to the fluid evacuation port 110. The liquid mixture may then be removed from the electrosurgical device 100 by the vacuum source and stored in the storage component for later removal.

In some non-limiting examples, the fluid aspiration port 165 may be formed at the distal end of an aspiration tube 160. The aspiration tube 160 may also form part of the aspirated fluid path 210. The aspiration tube 160 may be located within the shaft 135 or it may be located outside of and beneath the shaft 135. An aspiration tube 160 located outside of the shaft 135 may be in physical communication with an external surface of the shaft 135. In some examples, the aspiration tube 160 may have a fixed location with respect to the shaft 135. In some alternative examples, the aspiration tube 160 may be extendable in a distal direction with respect to the shaft 135. Extension of the extendable aspiration tube 160 may be controlled by means of an aspiration tube control device. As one non-limiting example, the aspiration tube control device may comprise a slide switch 130. The slide switch 130, in a first position (for example, in a proximal position), may cause the aspiration tube 160 to remain in a first or retracted position in which the aspiration port 165 is located essentially below the fluid discharge port 150. However, the slide switch 130 in a second position (for example in a distal position), may cause the aspiration tube 160 to extend in a distal direction to a fully extended position so that the aspiration port 165 is located distal from and beneath the fluid discharge port 150. In one example, the slide switch 130 may preferentially position the aspiration tube 160 in one of two positions, such as the retracted position and the fully extended position. It may be recognized, however, that the slide switch 130 may also permit the aspiration tube 160 to assume any position between the retracted position and the fully extended position. Regardless of the position of the aspiration tube 160 as disclosed above, the aspiration port 165 may be maintained at a location beneath a plane defined by the top surface of the diverter 155. In this manner, the diverter 155 is configured to prevent fluid emitted by the fluid discharge port 150 from directly being removed at the aspiration port 165.

FIG. 7 presents partial interior views of an electrosurgical device 200. In addition to the components disclosed above with respect to FIGS. 1-6, the electrosurgical device 200 includes an aspirated fluid path 210 that forms a fluid connection between the proximal fluid evacuation port 110 and the distal fluid aspiration port 165. Also illustrated are valve components 225 of the metering valve 125 and control components 230 of the aspiration tube such as, for example, a slide switch 130. Fluid discharge port 150, electrodes 145a,b, fluid aspiration port 165, and a portion of housing 105 are also illustrated in FIG. 7.

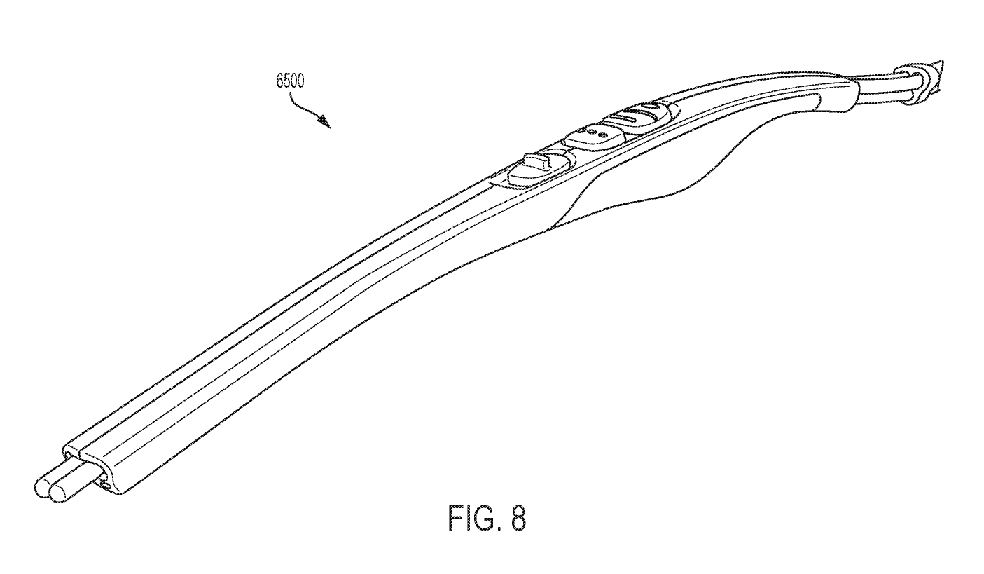

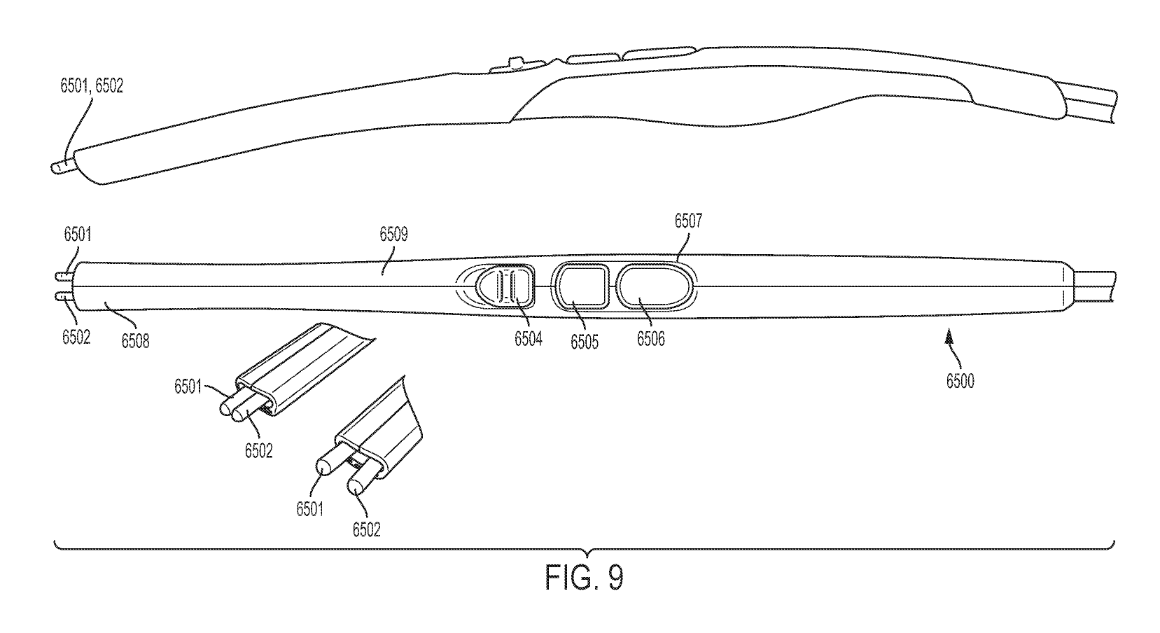

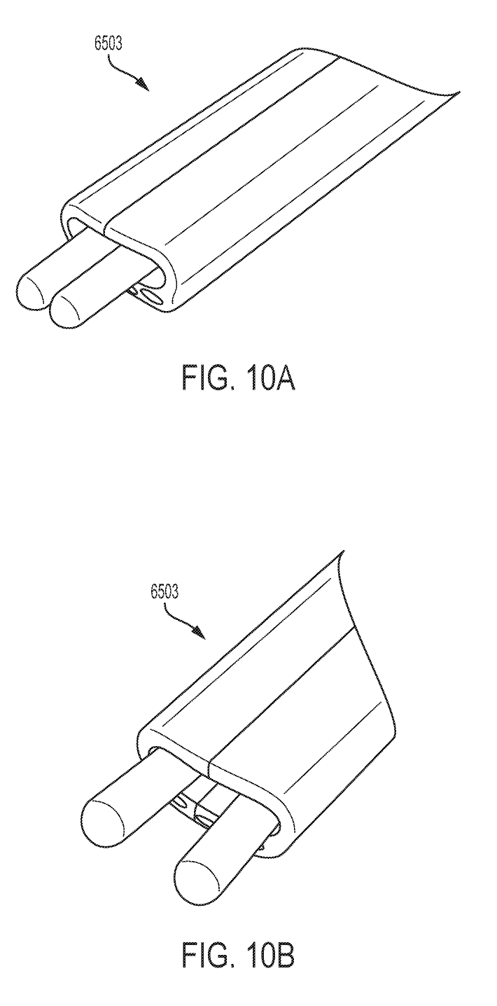

FIGS. 8-10 illustrate different views of another embodiment of a surgical device 6500 with spreadable bipolar electrodes 6501 and 6502 at the end effector 6503. In some embodiments as shown in these figures, a slider control may be provided in the handle to adjust the electrode aperture. For example, a sliding button 6504 may be provided to adjust the distance between the tips of the two electrodes 6501 and 6502. As shown in FIG. 9, the surgical device 6500 may have a length of 310 mm from the tip of the electrodes 6501 and 6502 to the end of the handle body 6509 as indicated in the figure. The distal end 6508 may have a width of 14 mm. The two electrodes 6501 and 6502 may have a minimum distance of 4 mm, as depicted in FIG. 10A, and a maximum distance of 8.5 mm, as depicted in FIG. 10B. Each of the two electrode probes 6501 and 6502 may have a diameter 3.5 mm. The sliding button 6504 controls the spreading of the electrodes 6501 and 6502 by sliding along the longitudinal direction a of the elongate handle body 6509. A power or coagulation button 6505, an irrigation button 6506, and a suction button 6507 are provided separately from the electrode spread button 6504.

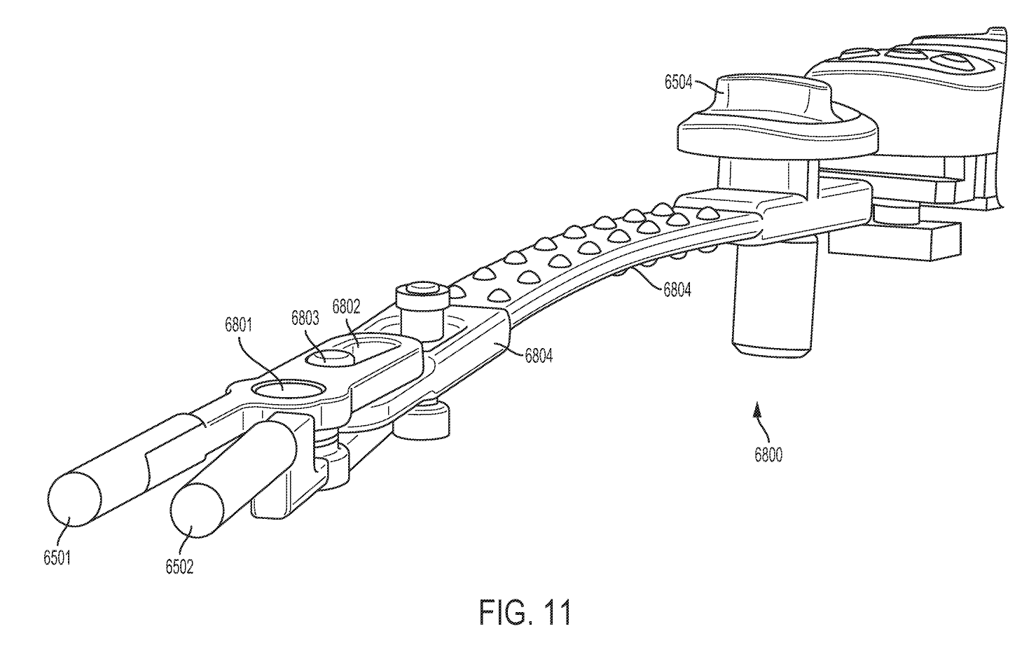

FIG. 11 depicts a view of one example control mechanism 6800 of the surgical device 6500 with the spreadable electrodes 6501 and 6502. The electrodes 6501 and 6502 are pivotably connected to each other by a connecting member 6801. A slot 6802 is provided on one electrode 6501. An elongate connector 6804 connects the electrode spread button at the proximal end and the electrode 6501 at the distal end. The connector 6804 includes a pin 6803 at the distal end, and the pin 6803 slidably fits in the slot 6802. When the pin 6803 slides along the slot 6802, the distance between the electrodes 6501 and 6502 is adjusted. For example, when the electrode spread button 6504 is slid backward towards the proximal end of the surgical device 6500, the connector 6804 pulls back the pin 6803 in the slot 6802, and the electrodes 6501 and 6502 are closed up. When the electrode spread button 6504 is slid forward towards the distal end of the surgical device 6500, the connector 6804 pushes forward the pin 6803 in the slot 6802, and the electrodes 6501 and 6502 spread farther from each other.

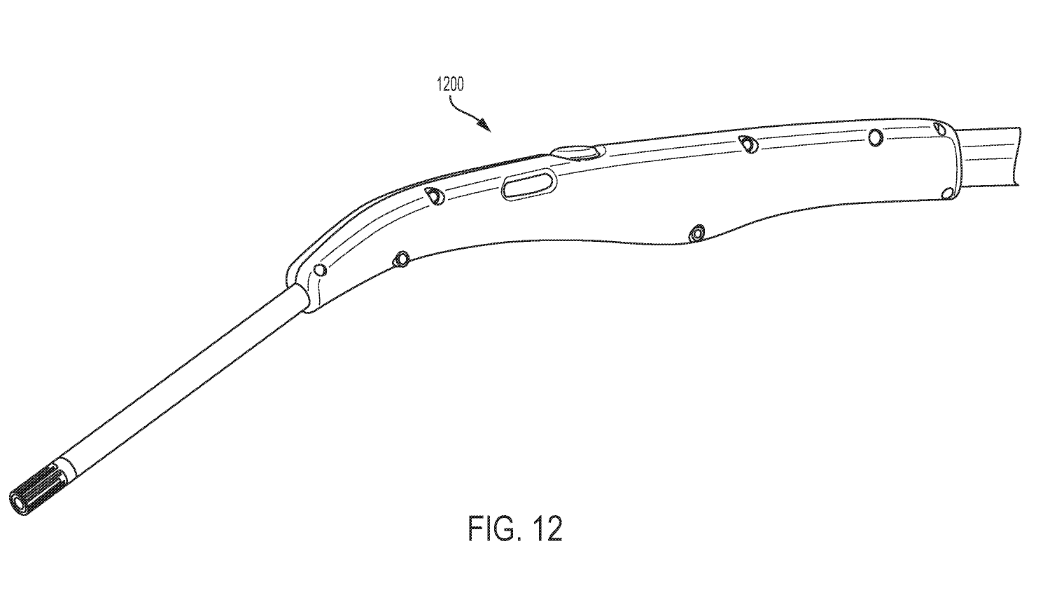

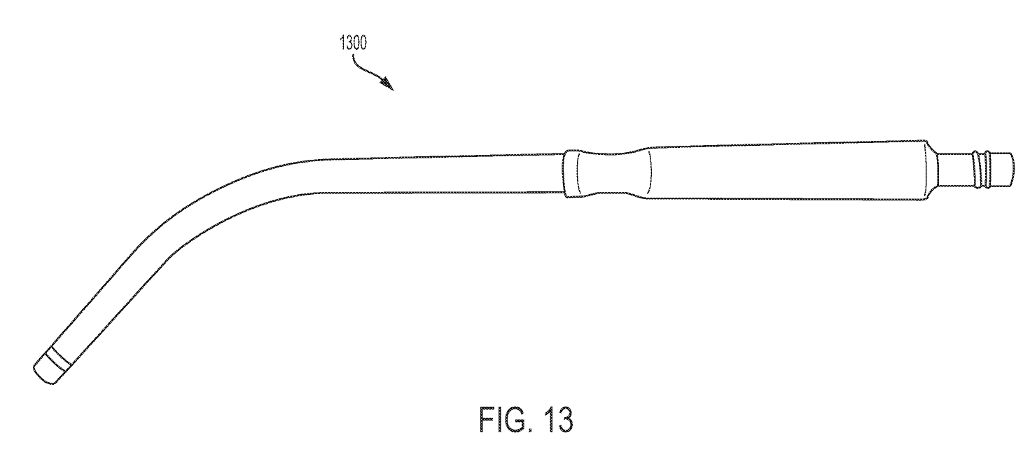

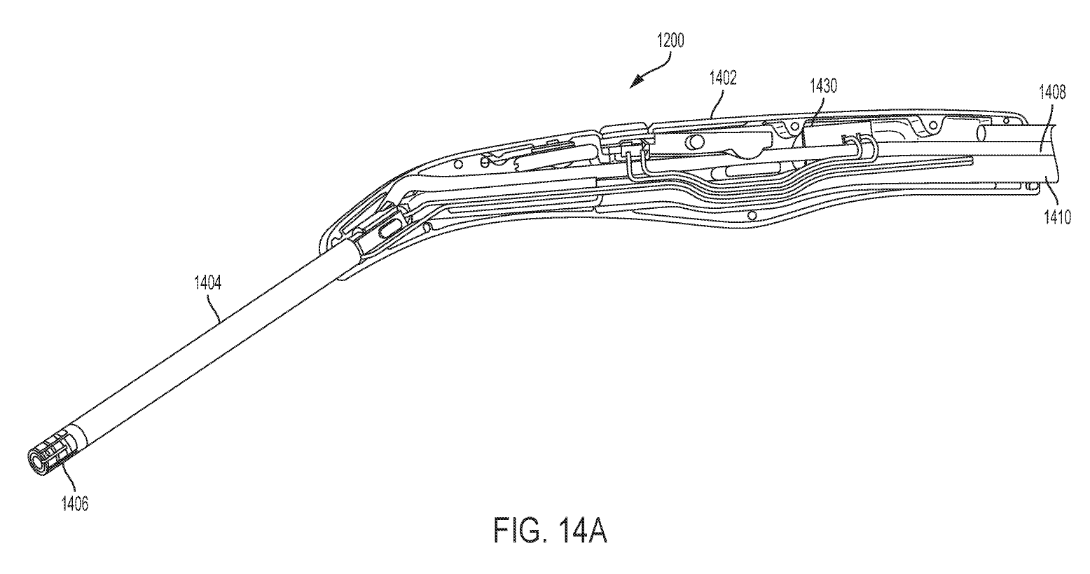

FIG. 12 depicts another aspect of an electrosurgical device 1200 configured to deliver a fluid to a surgical site and to remove fluid and other materials from such a site. It may be noted that the electrosurgical device 1200 bears a resemblance to a Yankauer suction device 1300 depicted in FIG. 13. A health care professional who may use the electrosurgical device 1200 may find such a similarly helpful in that the professional may already be used to handling the Yankauer device 1300.

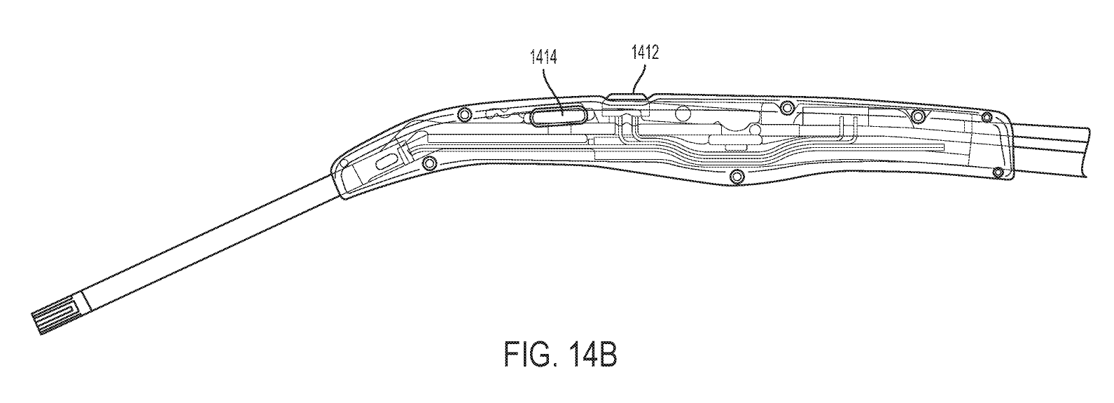

FIGS. 14A, B depict partial cross-sectional views of electrosurgical device 1200. Included in FIGS. 14A,B are a shaft assembly 1404, an end effector 1406, and a housing 1402. Additional components of the electrosurgical device 1200 include a fluid source path 1408 and a fluid aspiration path 1410. The fluid source path 1408 may provide a path for a fluid to flow through the electrosurgical device 1200 and enter the surgical site. This fluid, as disclosed above, may be used to permit a current flow between a positive and a negative electrode disposed on the end effector 1406. As a result, heated fluid or steam may be provided in the surgical site to cauterize tissue. Additionally, the fluid may be used to lubricate and clean the electrodes, thereby prevent fouling of the electrodes with a resultant reduction of efficiency. Further, the fluid or steam may be used to cleanse the surgical site of debris. The fluid aspiration path 1410 may permit a vacuum to be applied to the surgical site, thereby removing debris and excess water.

Additional aspects depicted in FIGS. 14A,B may include user control for the function of the electrosurgical device 1200. Such controls may include a fluid flow control 1414 and a combination power and fluid source control 1412. The combination control 1412 may be activated by a user to supply power to the electrodes on the end effector 1406 and at the same time permit a source of the fluid to flow through the electrosurgical device 1200. The fluid flow control 1414 may be adjusted by a user to regulate an amount of fluid flowing to the surgical site. In some aspects, the electrosurgical device 1200 may also include certain electronic memory devices such a device identification chip 1430. The identification chip 1430 may store information in any appropriate electronic form or format that may be accessed by medical device controller. The information may identify the type of medical device, a lot number, a serial number, and/or additional information related to proper or acceptable levels of power to be applied to the electrodes by the medical device controller, for example.

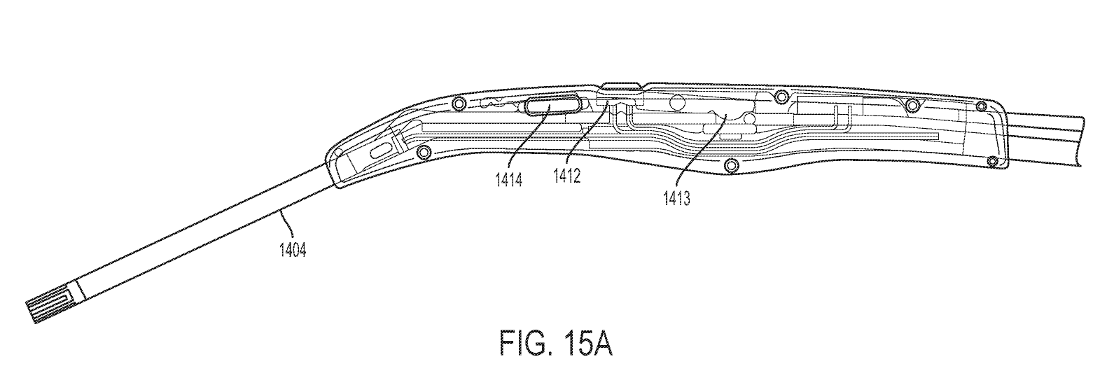

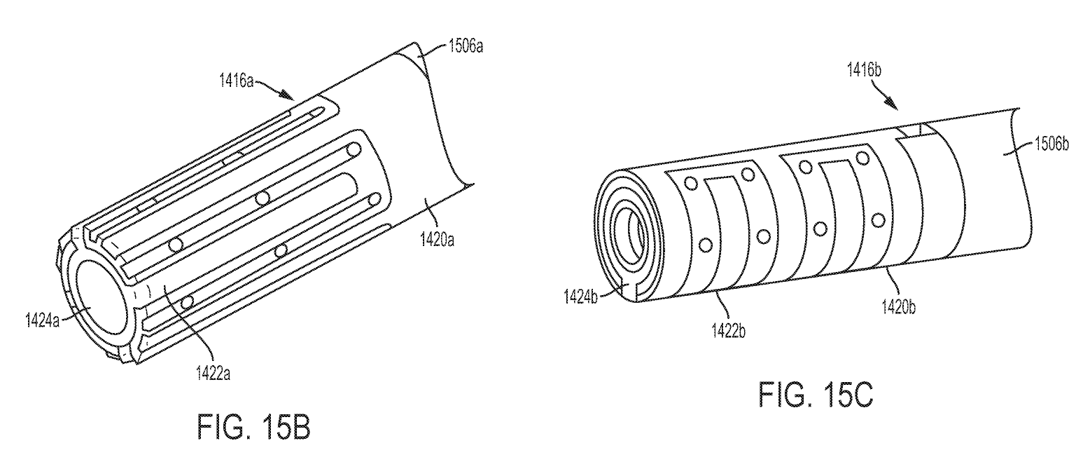

FIGS. 15A-C depict additional details of the electrosurgical device 1200. In FIG. 15A, along with the shaft assembly 1404 and the fluid flow control 1414, FIGS. 15A depicts some additional details of the combination control 1412, specifically a pinch valve 1413 that may allow or restrict the fluid flow to the fluid flow control 1414. FIGS. 15B and 15C depict alternative aspects of an end effector disposed at a distal end of the shaft assembly 1404.

FIG. 15B depicts a first end effector 1506a that is composed of interdigitated electrodes 1416a. The interdigitated electrodes 1416a are composed of a positive voltage electrode 1420a and a negative electrode 1422a. Each of the positive voltage electrode 1420a and a negative electrode 1422a comprise portions that are disposed on a surface of the first end effector 1506a and along a longitudinal axis of the first end effector 1506a. On a distal surface of the first end effector 1506a is an end ring 1424a that may be in electrical communication with the negative electrode 1422a.

FIG. 15C depicts a second end effector 1506b that is composed of multiple ring electrodes 1416b. The multiple ring electrodes 1416b are composed of a positive voltage electrode 1420b and a negative electrode 1422b. Each of the positive voltage electrode 1420b and a negative electrode 1422b comprise circular portions that are disposed on a surface of the first end effector 1506b and are orthogonal to a longitudinal axis of the first end effector 1506b. On a distal surface of the first end effector 1506b is an end ring 1424b that may be in electrical communication with the positive electrode 1422b.

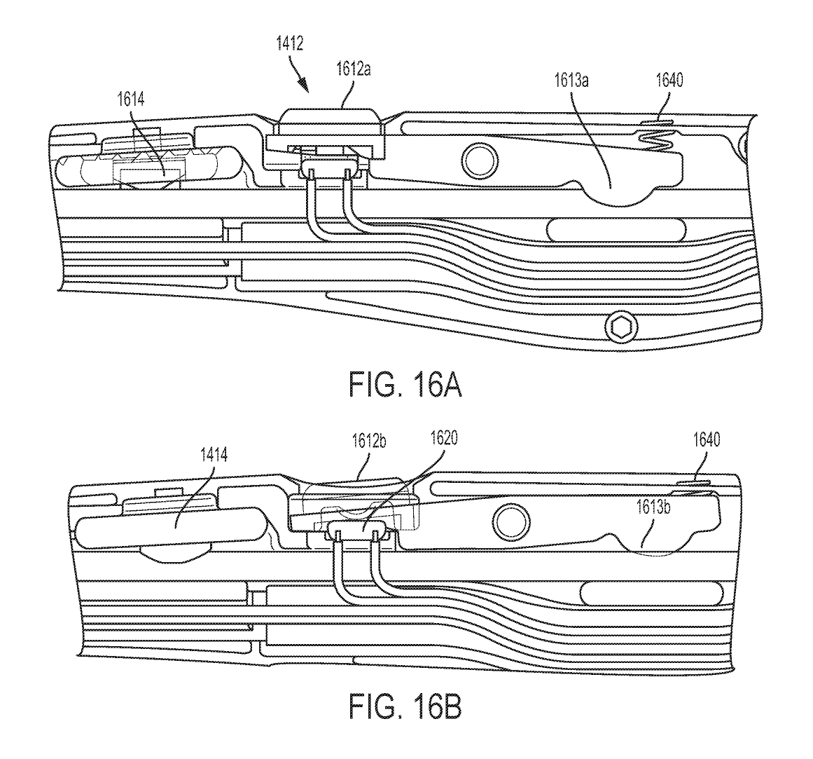

FIGS. 16A,B depict some of the operations of the combination control 1412. The combination control may include a button that is normally in a raised position (1612a). In this position, an electrode power switch 1620 may be in an “off” configuration, preventing power from being supplied to the electrodes. At the same time, a pinch valve may be in a closed position 1613a, thereby constricting a fluid path and restricting fluid from flowing to the fluid flow control 1414. When the button on the combination control is depressed by a user, to a depressed position (1612b), the electrode power switch 1620 may be placed in an “on” configuration, thereby permitting electrical power to be delivered to the electrodes. At the same time, the pinch valve may be placed in an open position 1613b, thereby permitting fluid to flow to the fluid flow control 1414. In this manner, the electrodes receive the fluid when activated and the user is not required to activate separate controls. The combination control 1412 may be normally maintained in the raised or off position 1612a by means of a bias spring 1640.

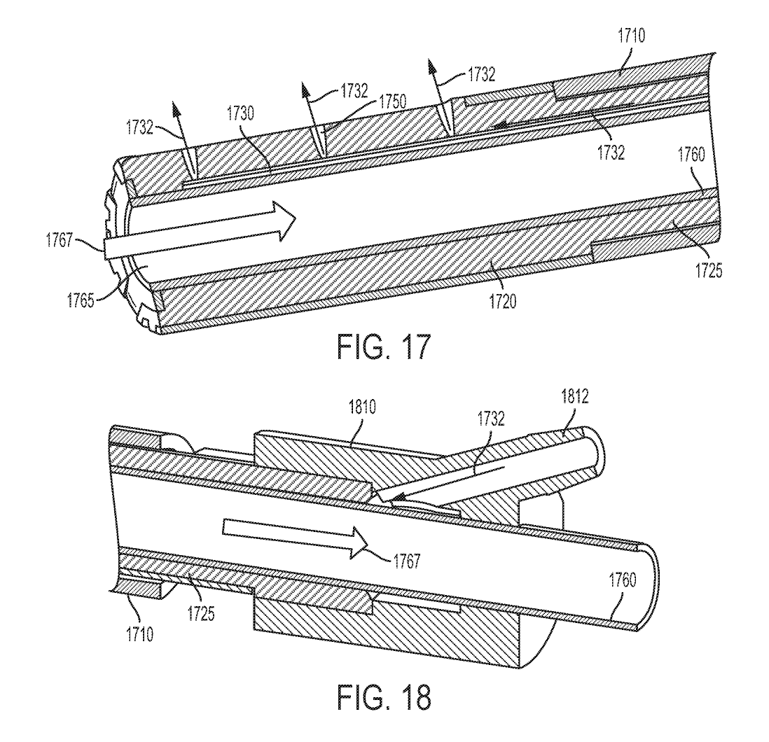

FIGS. 17 and 18 depict fluid flow paths through the electrosurgical device 1200. FIG. 17 depicts fluid flow at a distal end of the shaft assembly 1404 and FIG. 18 depicts fluid flow at a proximal end of the shaft assembly 1404. FIG. 17 depicts a cross sectional view of the end effector. The end effector may be composed of an end effector body 1720 and an end effector body extension 1725. The end effector body 1720 may be disposed at the distal end of a shaft 1710, and the end effector body extension 1725 may disposed at least in part within the shaft 1710. An aspiration tube 1760 may be disposed within an interior of both the end effector body 1720 and the end effector body extension 1725. The aspiration tube 1760 may be in fluid communication with an aspiration port 1765 disposed at the distal end of the end effector body 1720. Fluid and additional debris may flow (aspiration flow 1767) through the aspiration port 1765 and be transported via the aspiration tube 1760 to a vacuum source in fluid communication with the aspiration tube 1760 and configured to remove such material.

Fluid, supplied by a fluid source, may flow through the combination control 1412 and the fluid flow control 1414. The fluid may be directed to a fluid flow space 1730 that may be created between an exterior surface of the aspiration tube 1760 and an interior surface of both the end effector body 1720 and the end effector body extension 1725. The fluid source flow 1732 may be directed through the fluid flow space 1730 and exit via fluid discharge ports 1750 disposed in the end effector body 1720.

FIG. 18 depicts fluid flow through the proximal end of the shaft assembly 1404. In FIG. 18, a portion of the aspiration tube 1760 is depicted as disposed within a fluid adapter 1810. As depicted in FIG. 18, the aspiration tube 1760 is disposed within the end effector body extension 1725, and the end effector body extension 1725 is disposed within the shaft 1710, forming a set of nested tubular components. The aspiration flow 1767 continues through the interior of the aspiration tube 1760 in a proximal direction. The fluid source flow 1732 enters the fluid adapter 1810 via an adapter source port 1812. The adapter source port 1812 may receive the fluid that is regulated by the fluid flow control 1414.

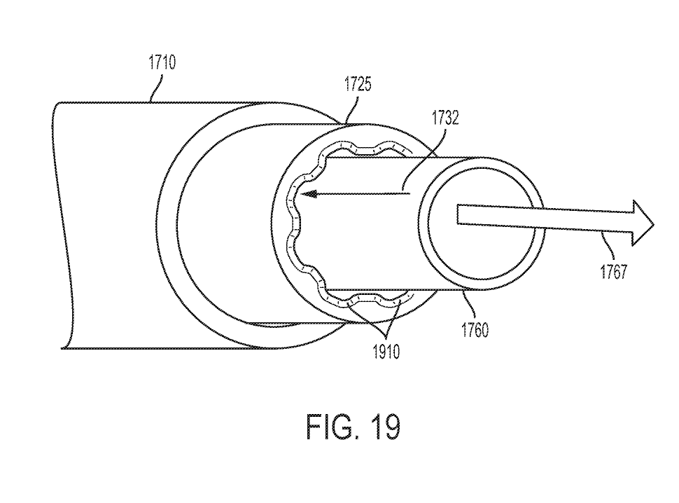

FIG. 19 depicts an expanded view of the proximal end of the shaft assembly and depicts in detail the aspiration flow 1767 and the fluid source flow 1732. As disclosed above, the shaft 1710, end effector body extension 1725, and the aspiration tube 1760 are nested within each other. The fluid sourced at the adapter source port 1812 is direct through a series of fluid vents 1910 at the proximal end of the end effector body extension 1725. Such fluid vents 1910 permit access of the fluid to the fluid flow space 1732 disposed between the interior of the end effector body extension 1725 and the exterior of the aspiration tube 1760.

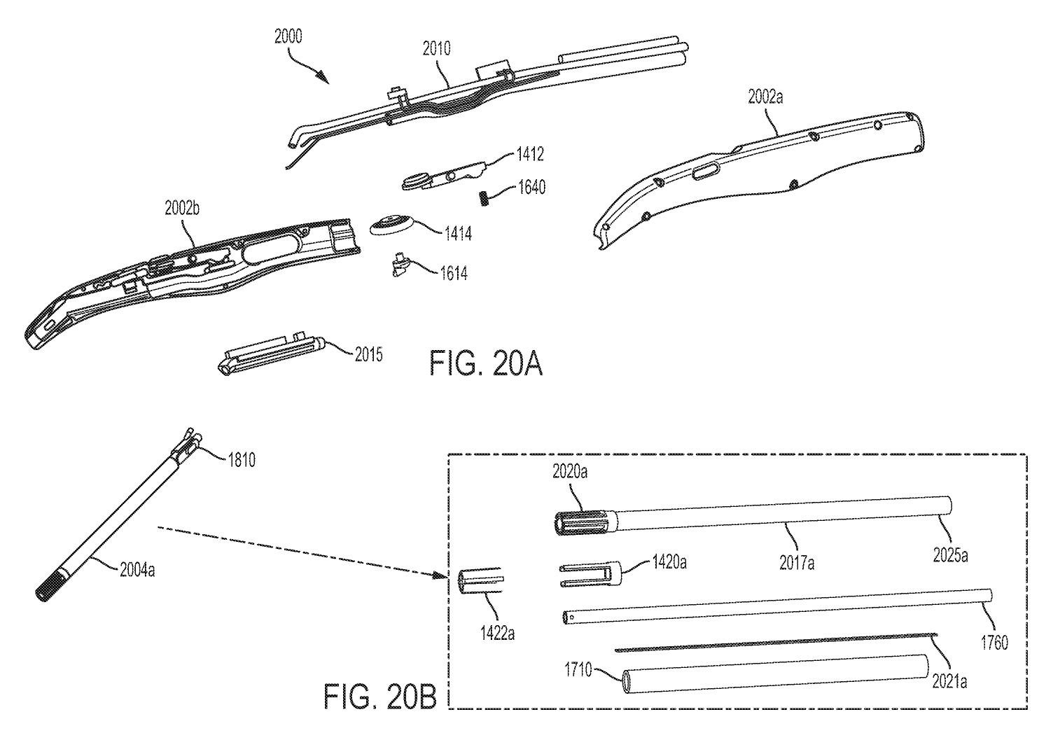

FIGS. 20A,B depict exploded views of the electrosurgical device 1200. FIG. 20A depicts an exploded view of a handle assembly 2000 and a shaft assembly 2004a for a device having an end effector as depicted in FIG. 15B. Many of the components depicted in FIG. 20A have been disclosed and described above. Additional components include a first housing portion 2002a and a second housing portion 2002b that, together, form the housing 1402 of the electrosurgical device 1200. Also depicted are an internal handle assembly 2010, which may be composed of various electrical and fluidic components, as are disclosed below. An aspirator adapter 2015 is also depicted. The aspirator adapter 2015 is configured to fluidically couple the fluid adapter 1810 with a fluid path to a vacuum source to remove material from the surgical site.

FIG. 20B depicts an exploded view of the components of a first shaft assembly 2004a. First shaft assembly 2004a is an aspect of a shaft assembly 1404 and which is configured to be used with an interdigitated end effector 1506a. In addition to the interdigitated electrodes 1420a and 1422a, the first shaft assembly 2004a includes a shaft 1710 and an aspiration tube 1760. Further, the first shaft assembly 2004a is composed an end effector unit 2017a composed of an end effector body 2020a and an end effector body extension 2025a. I may be recognized that the end effector body 2020a and an end effector body extension 2025a may be fabricated together as a single end effector unit 2017a. Alternatively, the end effector body 2020a and the end effector body extension 2025a may be fabricated separately and later joined together to form the end effector unit 2017a. An additional positive electrode conductor 2021a is also depicted.

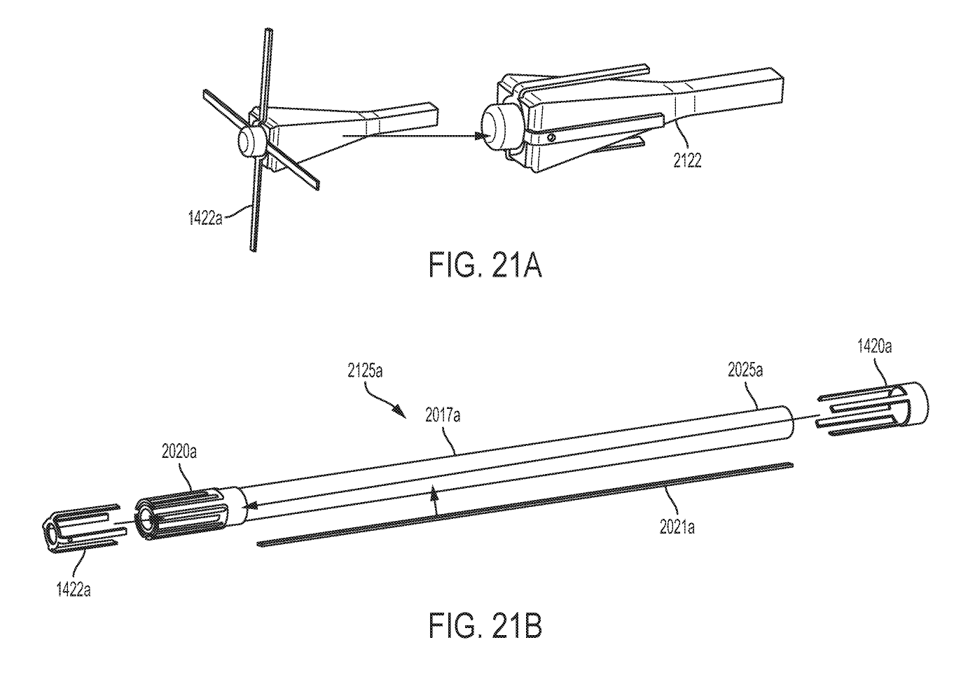

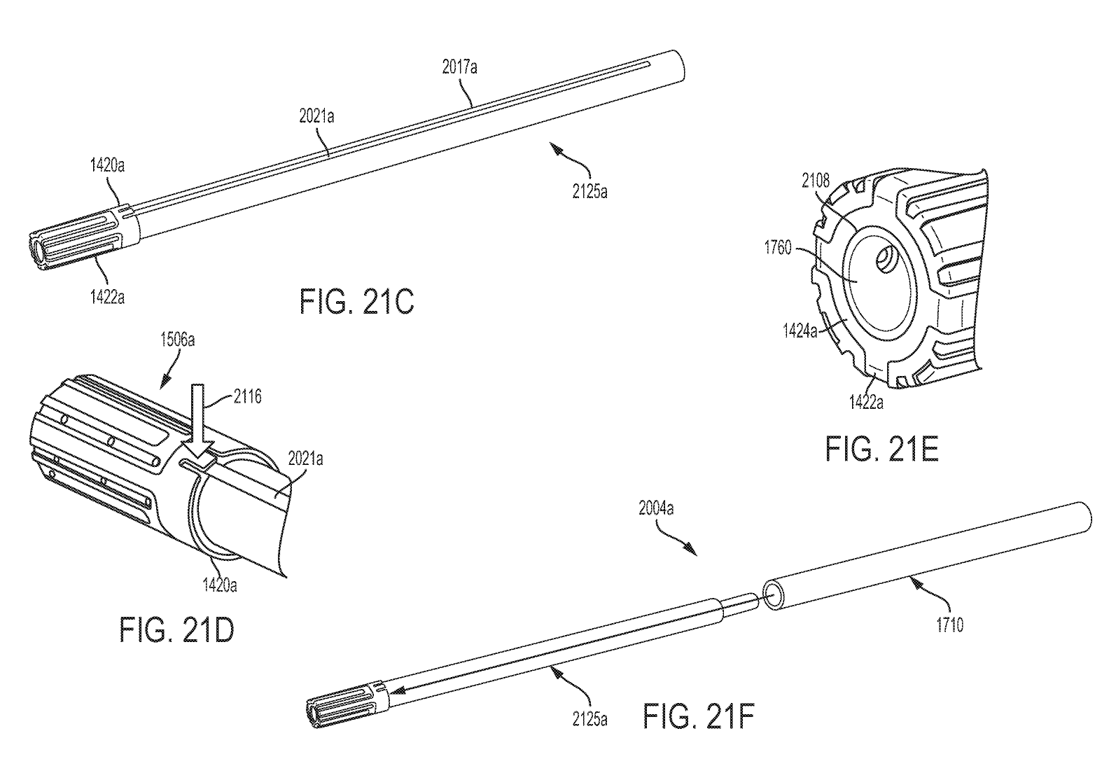

FIGS. 21-25 are assembly drawings directed to one aspect of a method to assemble the electrosurgical device 1200 from components depicted in FIGS. 20A,B.

FIGS. 21A-F are assembly drawings depicting one aspect of a process to fabricate first shaft assembly 2004a. FIG. 21A depicts the formation of a negative voltage (or ground) electrode 1422a. A planar form of the negative electrode 1422a may be fabricated, for example from sheet stock. The negative electrode 1422a may include legs that, when assembled, are disposed on a surface of the end effector body 2020a and extend parallel to a longitudinal axis thereof. The negative electrode 1422a may be placed on a mandrel 2122 and the legs of the negative electrode 1422a may be bent in an appropriate manner for assembly. In this manner, the legs of the negative electrode 1422a may be bent so that they may be disposed on an outer surface of the end effector body 2020a and extend parallel to a longitudinal axis thereof upon being fixed to the end effector body 2020a.

The end effector assembly 2125a includes an end effector body 2020a and an end effector body extension 2025a (the end effector body 2020a and the end effector body extension 2025a together forming the end effector unit 2017a). The end effector body 2020a may include one or more fluid discharge ports (unlabeled) that may permit a fluid to flow on the exterior surface of the end effector body 2020a and contact the electrodes 1420a and 1422a. The end effector body 2020a may include recesses or ridges on its outer surface and disposed along a longitudinal axis thereof and further configured to receive and stabilize the positions of the legs of both of the negative electrode 1422a and the positive electrode 1420a. The negative electrode 1422a may slidably engage the end effector body 2020a from its distal end. The positive electrode 1420a may slidably engage the end effector body 2020a via the end effector body extension 2025a, proceeding in a proximal to distal direction. The end effector body extension 2025a may also include a channel on an outer surface and configured to receive the positive electrode conductor 2021a.

FIGS. 21C and 21D depict expanded views of the assembled end effector assembly 2125a including the positive electrode 1420a, the negative electrode 1422a, the end effector unit 2017a, and the positive electrode conductor 2021a. In some aspects, the positive electrode 1420a, the negative electrode 1422a, and the positive electrode conductor 2021a may be glued to the end effector body 2020a and the end effector body extension 2025a, respectively. FIG. 21D particularly depicts the connection 2116 between the positive electrode conductor 2021a and the positive electrode 1420a on the end effector 1506a. This connection 2116 may be made by any appropriate means including, without limitation, welding, soldering, or using an appropriate electrically conducting adhesive.

FIG. 21E depicts the distal end of the end effector 1506a. In particular, FIG. 21E illustrates the distal end of the aspirator tube 1760 disposed within the end effector body 2020a. FIG. 21E also depicts the placement of end ring 1424a on a distal face of the end effector body 2020a. Additionally, it may be seen that the end ring 1424a is electrically connected 2108 to aspirator tube 1760. It may be understood that the aspirator tube 1760, as used in end effector assembly 2125a, is electrically conducting and acts as well to conduct electrical current from the electrodes to a current ground in the power source.

FIG. 21F depicts the final stage of the fabrication of the shaft assembly 2004a, in which the end effector assembly 2125a slidably engages the shaft 1710.

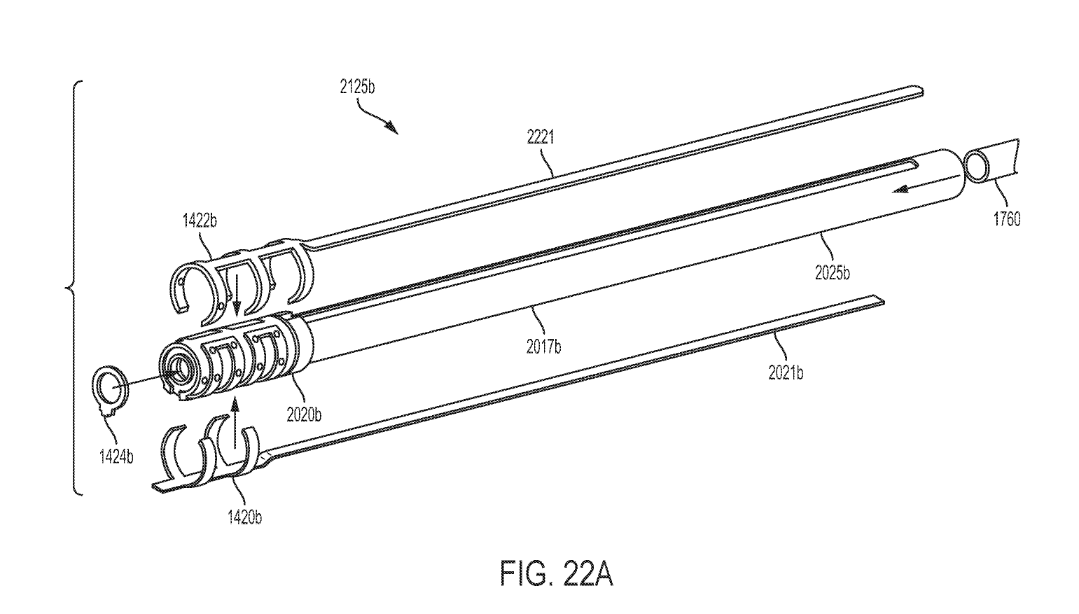

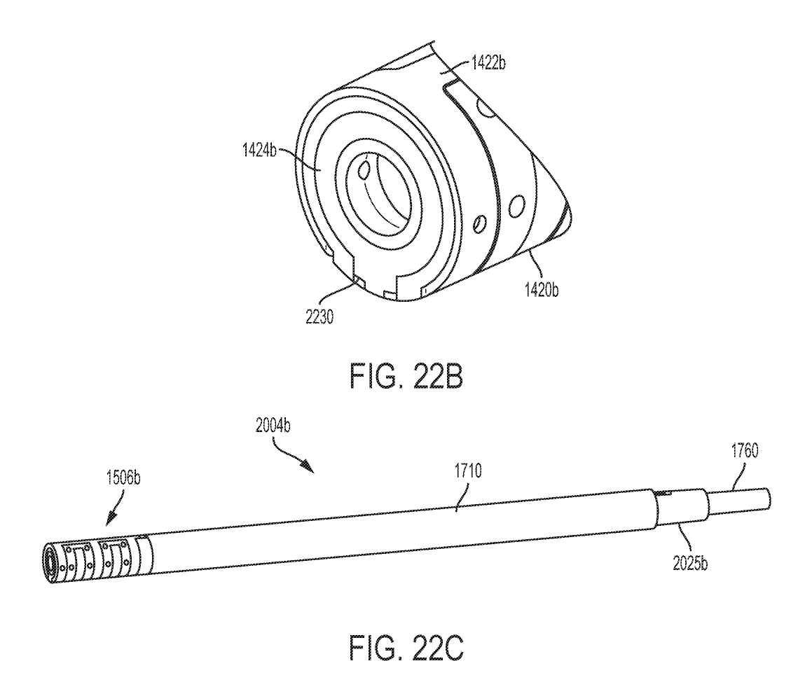

FIGS. 22A-C depict an aspect of the fabrication of the second shaft assembly 2004b. Second shaft assembly 2004b is particularly designed for the fabrication of an electrosurgical device 1200 having a ring end effector 1506b.

The end effector assembly 2125b includes an end effector body 2020b and an end effector body extension 2025b (the end effector body 2020b and the end effector body extension 2025b together forming the end effector unit 2017b). The end effector body 2020b may include one or more fluid discharge ports (unlabeled) that may permit a fluid to flow on the exterior surface of the end effector body 2020b and contact the electrodes 1420b and 1422b. The end effector body 2020b may include recesses or ridges on its outer surface and disposed along a longitudinal axis thereof and further configured to receive and stabilize the positions of the legs of both of the negative electrode 1422b and the positive electrode 1420b. It may be recognized that the legs of negative electrode 1422b and the legs of the positive electrode 1420b are curved to engage the circumferential surface of the end effector body 2020b. The legs of the negative electrode 1422a may sufficiently pliable to be snap-fit onto the outer surface of the end effector body 2020b. Similarly, the legs of the positive electrode 1420b may be sufficiently pliable to be snap-fit onto the outer surface of the end effector body 2020b.

Positive electrode 1420b may be affixed to a positive electrode conductor 2021b that may extend along an outer length of end effector body extension 2025b. Similarly, negative electrode 1422b may be affixed to a negative electrode conductor 2221 that may extend along an outer length of end effector extension 2025b. In some aspects, the positive electrode 1420b and positive electrode conductor 2021b may be fabricated as a single unit. In alternative aspects, the positive electrode 1420b and positive electrode conductor 2021b may be fabricated as separate components and may be connected by any appropriate means, including, without limitation, welding, soldering, or using an appropriate electrically conducting adhesive. In some aspects, the negative electrode 1422b and negative electrode conductor 2221 may be fabricated as a single unit. In alternative aspects, the negative electrode 1422b and negative electrode conductor 2221 may be fabricated as separate components and may be connected by any appropriate means, including, without limitation, welding, soldering, or using an appropriate electrically conducting adhesive. The end effector body extension 2025a may also include a first channel on an outer surface and configured to receive the positive electrode conductor 2021b. The end effector body extension 2025a may also include a second channel on an outer surface and configured to receive the negative electrode conductor 2221. Aspiration tube 1760 may be inserted into the interior of the end effector unit 2017b.

In some aspects, the positive electrode 1420b, the negative electrode 1422b, the positive electrode conductor 2021b, and the negative electrode conductor 2221 may be glued to the end effector body 2020b and the end effector body extension 2025b, respectively.

FIG. 22B depicts the distal end of the end effector 1506b. In contrast to the depiction of the distal end of end effector 1506a in FIG. 21E, the distal end of the aspirator tube 1760 does not extend to the distal end of end effector 1506b. Instead, a non-conducting portion of the end effector body 2020b forms the evacuation port 1765. FIG. 22B also depicts the placement of end ring 1424b on a distal face of the end effector body 2020b. In this aspect, the end ring 1424b is electrically connected 2230 to the positive electrode 1420b. It may be recognized that such a connection may permit the distal face of the end effector 1506b to apply a cauterizing power to a tissue.

FIG. 22C depicts a perspective view of the assembled shaft assembly 2004b including the end effector 1506b, the shaft 1710, a proximal extension of the end effector body extension 2025b, and the distal end of the aspiration tube 1760. It may be understood that, as similarly depicted in FIG. 21F, the assembled end effector assembly 2125b may be slidably received by the shaft 1710.

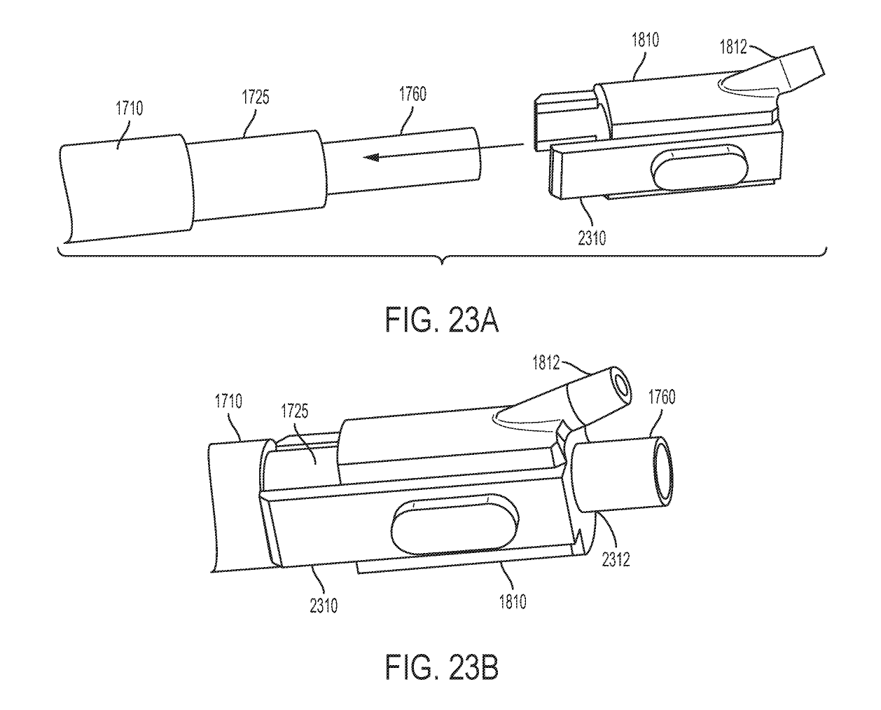

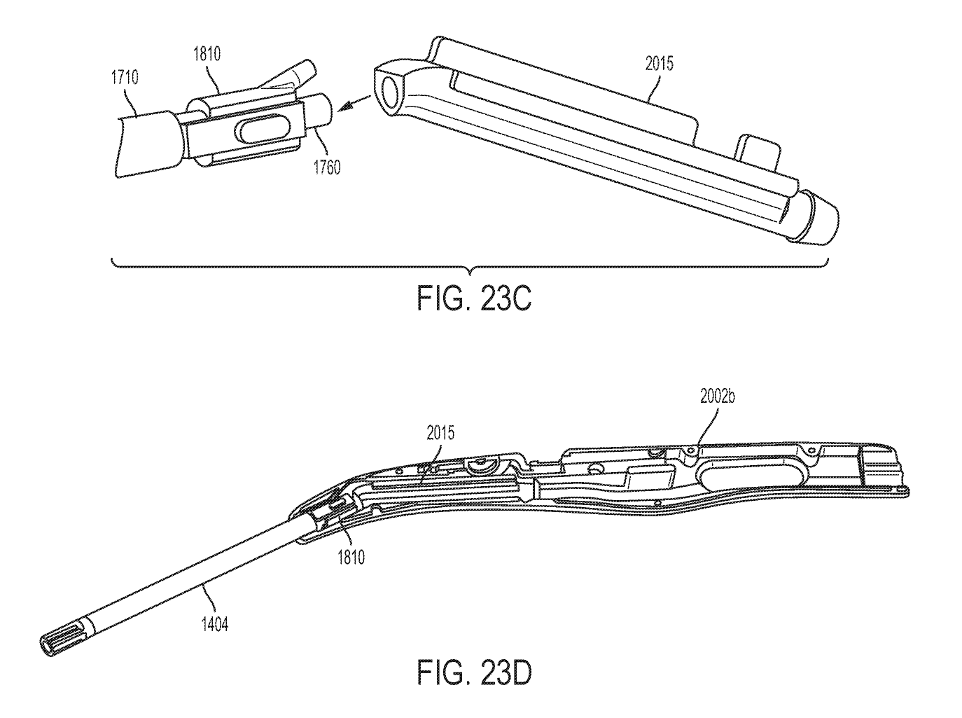

FIGS. 23A-D depict an aspect of an assembly procedure of components related to fluid flow in the electrosurgical device 1200. In some aspects, an adhesive material may be placed around an outer surface at a proximal end of the end effector body extension 1725. The fluid adapter 1810 may be slidably moved in a distal direction over both a proximal end of the aspiration tube 1760 and the proximal end of the end effector body extension 1725. The fluid adapter 1810 may be positioned so that the fluid adapter tabs 2310 abut a proximal edge of the shaft 1710. In this manner an interior space of the fluid adapter 1810 may permit a fluid to flow in a distal direction through the adapter source ports 1812 and enter the fluid vents 1910 of the end effector body extension 1725. The fluid adapter 1810 may be positioned so that at least a portion of the aspiration tube 1760 extends in a proximal direction beyond the body of the fluid adapter 1810 (see FIG. 23B). Additional adhesive may be applied (FIG. 23B, arrows) at the proximal edge of the fluid adapter and encircling an outer surface of the aspiration tube 1760. Such an adhesive may result in a fluid-tight seal, thereby preventing fluid flowing through the fluid adapter 1810 from seeping around the exterior surface of the aspiration tube 1760. It may be understood that additional or alternative methods may be used to form a fluid-tight seal at the fluid adapter 1810/aspiration tube 1760 junction including, for example, a chemical weld, or an o-ring.

FIGS. 23C,D depict an aspect of a method of affixing a distal portion of an aspiration adapter 2015 to the proximal portion of the aspiration tube 1760. In one aspect, an adhesive may be applied to the outer surface of the aspiration tube 1760 and the aspiration adapter 2015 may slidably engage the adhesive-covered portion of the aspiration tube 1760. In some aspects, the combined unit composed of the shaft assembly 1404, fluid adapter 1810, and aspiration adapter 2015 may be placed into an interior space of the second housing portion 2002b while the adhesive applied to the outer surface of the aspiration tube 1760 remains in an uncured state. Such a method may result in the components of the combined unit being properly aligned with each other, as well as to assure an effective fit of the combined unit within the second housing portion 2002b.

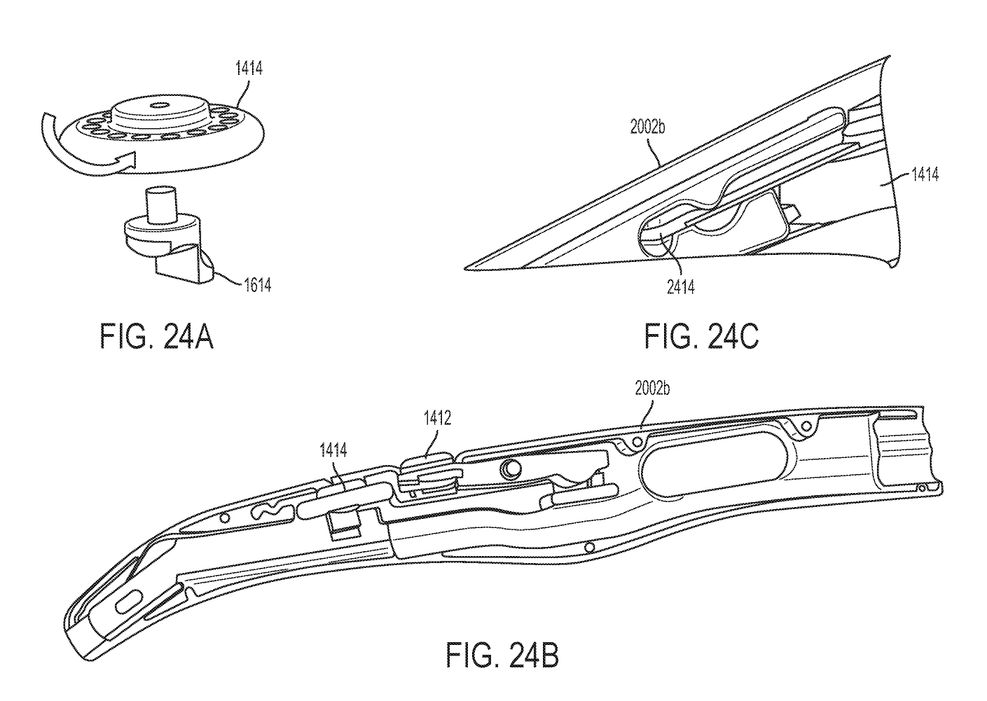

FIGS. 24A-C depict as aspect of a method of inserting the fluid flow control 1414 into the second housing portion 2002b of the electrosurgical device 1200. The fluid flow control 1414 may be placed onto or screwed onto a top portion of the fluid flow control mount 1614. In some aspects, the fluid flow control mount 1614 may form a pinch valve configured to compress a flexible tube that may conduct the fluid in a distal manner from the fluid source pinch valve 1413. The combination of the fluid flow control 1414 with the fluid flow control mount 1614 may be inserted into a bracket space within the interior space of the second housing portion 2002b. A retaining leaf spring 2414 may be inserted into the bracket space and may be used to provide a bias force against the fluid flow control 1414.

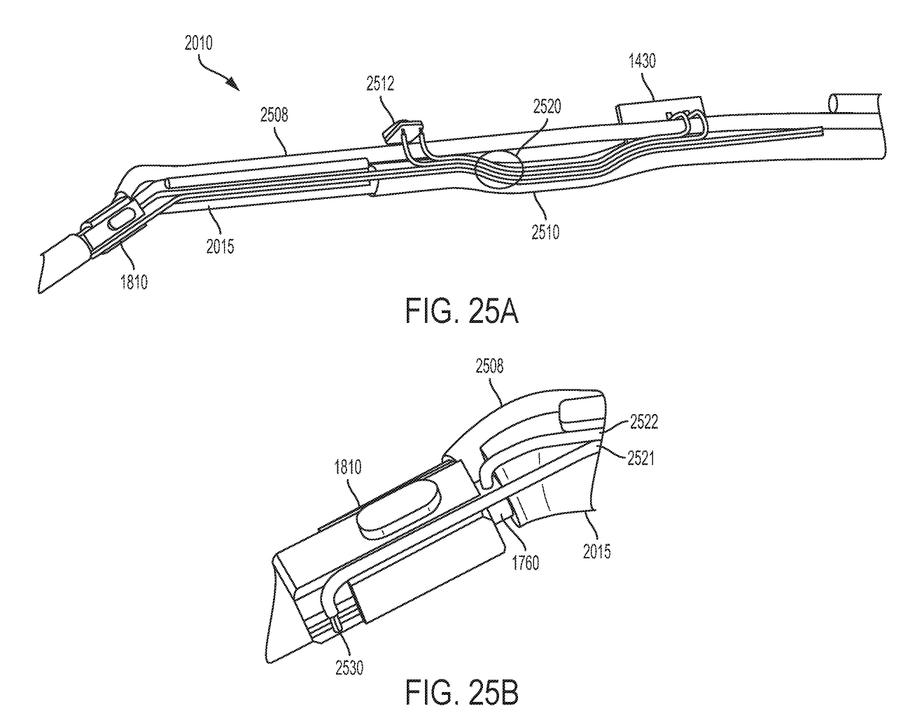

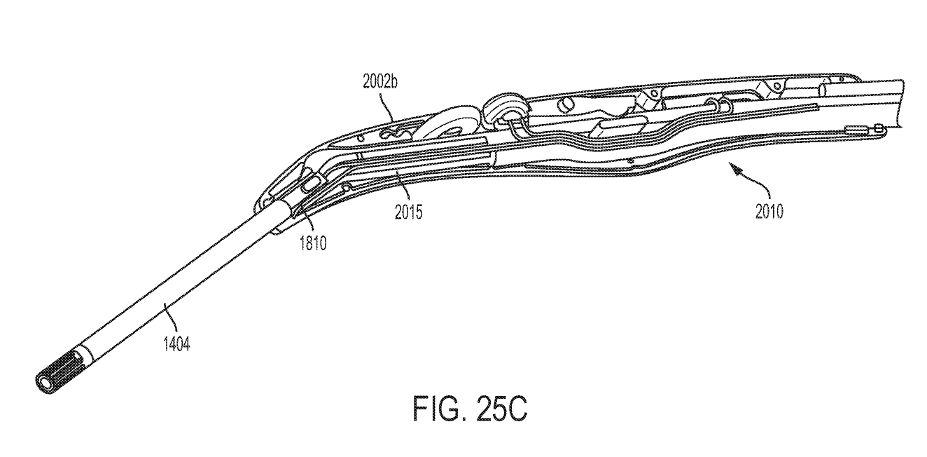

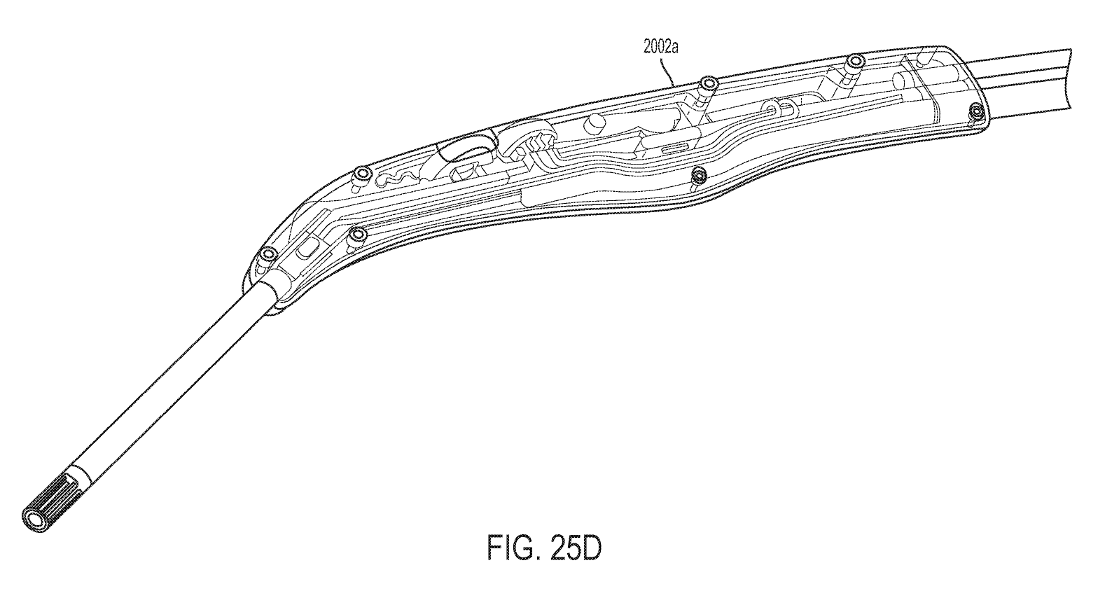

FIGS. 25A-D depict an aspect of some methods for completing the assembly of the electrosurgical device 1200.

Internal handle assembly 2010 may be composed of both fluidic and electrical components. The fluidic components may include a fluid source path 2508 and a fluid evacuation path 2510. The electrical components may include an electrical conductor bundle 2520. The electrical conductor bundle 2520 may include electrical conductors that may contact a power control switch 2512. The power control switch 2512 may be activated when a user depresses the button portion of the combination control 1412. The electrical conductor bundle 2520 may also include electrical conductors that may contact the device identification chip 1430. Additional electrical conductors may provide an electrical path to permit information stored on the device identification chip 1430 to be received by the device control system. Further electrical conductors may supply RF power and provide an RF power return to the electrodes.

The fluid source path 2508 may be fluidically coupled to the adapter source port 1812 of the fluid adapter 1810. The fluid evacuation path 2510 may also be coupled to a proximal coupling of the aspiration adapter 2015. Fluidical coupling may be accomplished by any appropriate means including chemical welding or the application of an adhesive.

A positive RF power conductor 2521 may be electrically coupled to the positive electrode of the end effector. A negative RF power conductor 2522 may be electrically coupled to the negative electrode of the end effector. FIG. 25B specifically depicts an example of such electrical coupling for an electrosurgical device 1200 having an interdigitating end effector 1506a. As disclosed above with respect to FIGS. 21A-F, the positive electrode 1420a of the interdigitating end effector 1506a is electrically coupled to a positive electrode conductor 2021a that spans a length of the end effector body extension 2025a from the distal end (at the end effector 1506a) to the proximal end. The positive RF power conductor 2521 may be electrically coupled to the positive electrode conductor 2021a at a positive RF contact point 2530. The positive RF contact point 2530 may be fabricated according to any appropriate method including, for example, welding. FIG. 25B also depicts that the negative RF power conductor 2522 may be electrically coupled to the aspiration tube 1760. It may be recalled with respect to the disclosure of the interdigitating end effector 1506a that the negative electrode 2022a is electrically coupled to the aspiration tube 1760. FIG. 25B illustrated that a negative or ground power connection may be made between the aspiration tube 1760 and the negative RF power conductor 2522.

As disclosed above with respect to FIG. 23D, a combined unit composed of the shaft assembly 1404, fluid adapter 1810, and aspiration adapter 2015 may be placed into an interior space of the second housing portion 2002b while the adhesive applied to the outer surface of the aspiration tube 1760 remains in an uncured state. Such a method may result in the components of the combined unit being properly aligned with each other, as well as to assure an effective fit of the combined unit within the second housing portion 2002b. It may be recognized that once the adhesives applied to the components of the combined unit have cured, the combined unit may be removed from the second housing portion 2002b in order to assemble the components of the internal handle assembly 2010 therewith. The completed assembly, composed of the shaft assembly 1404, fluid adapter 1810, aspiration adapter 2015, and the internal handle assembly 2010 may be returned to the interior space of the second housing portion 2002b. FIG. 25D depicts the final assembly in which the first housing portion 2002a (in transparent perspective) is then attached to the second housing portion 2002b.

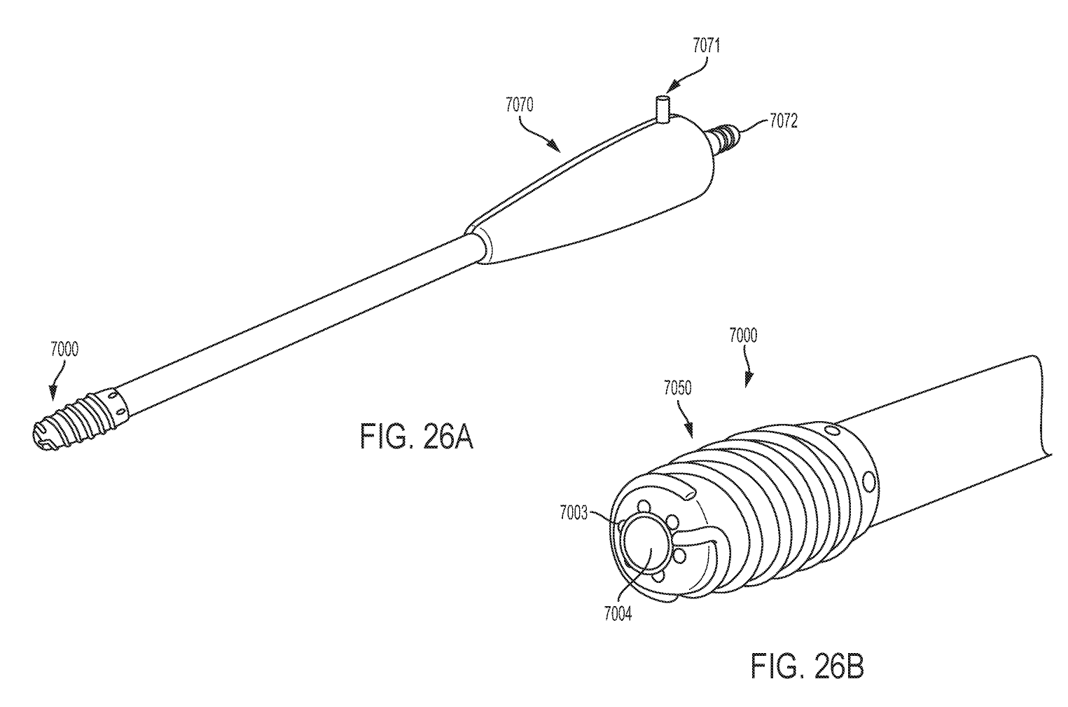

In another aspect, FIG. 26A depicts a surgical device 7070 having an end effector 7000 composed of bipolar electrodes arranged in a helical configuration following the contour of the tip 7050 of the end effector 7000. An irrigation fluid inlet 7071 and a suction outlet 7072 are provided at the proximal end of the surgical device 7070. The control buttons, such as the power or coagulation button, the irrigation button, and the suction button are not illustrated. FIG. 26B is a closer view of the end effector 7000. As shown in FIG. 26B, irrigation lumens 7003 may be provided at the distal face of the tip 7050. In some aspects, the irrigation lumens 7003 may be one or more fluid outlets surrounding a suction lumen 7004.

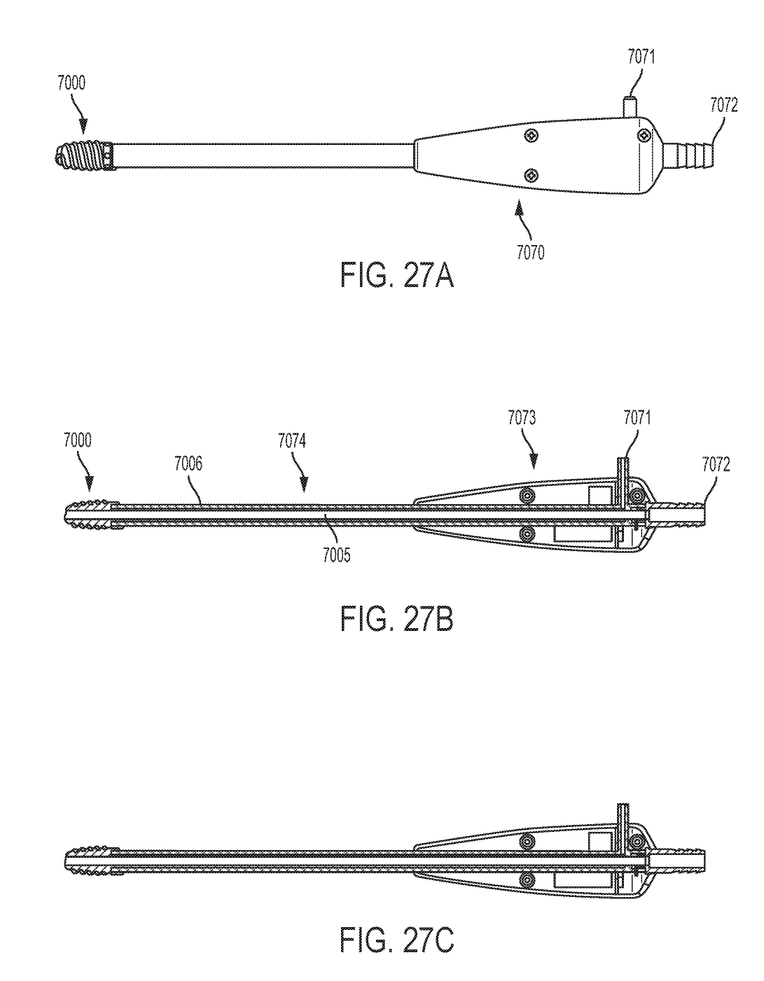

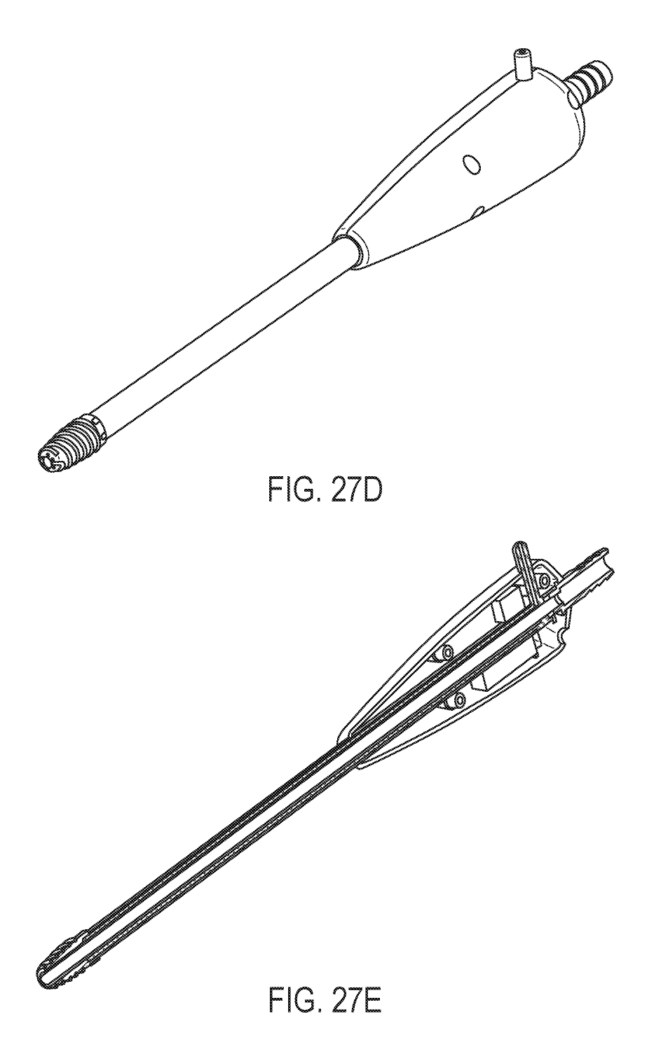

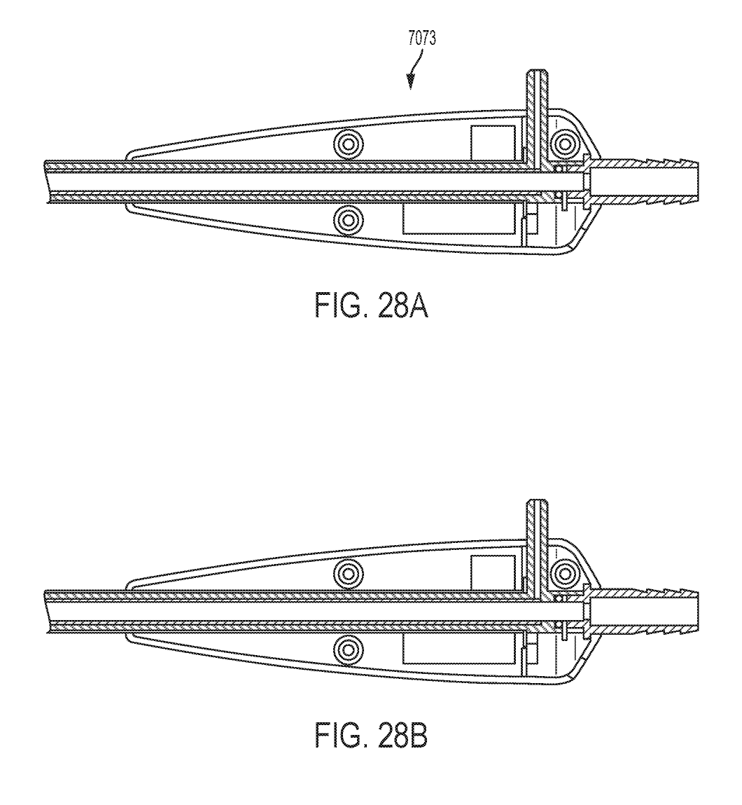

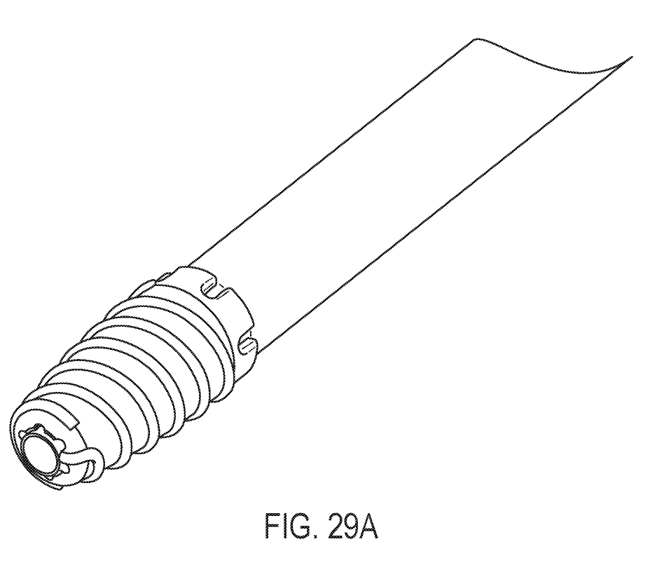

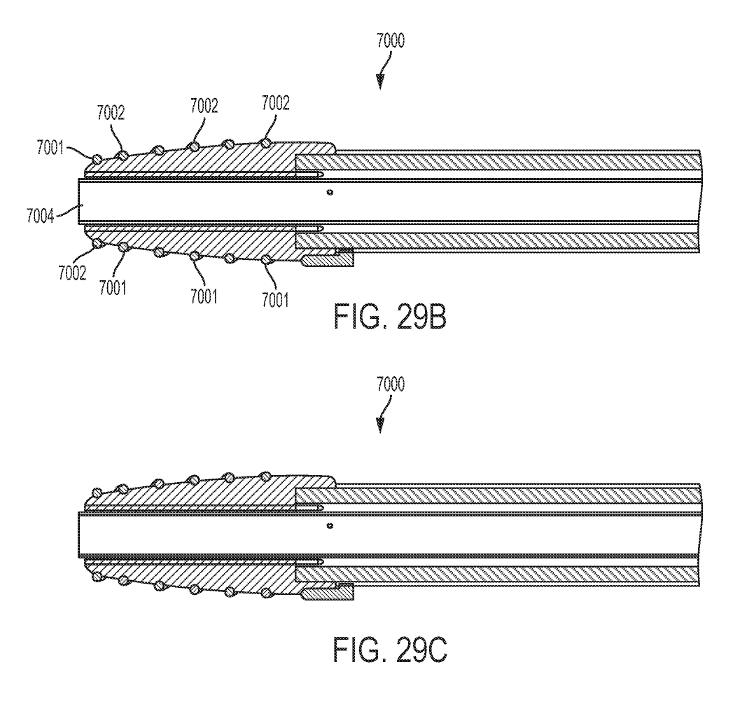

FIG. 27A illustrates another view of the surgical device 7070 shown in FIG. 26A. FIG. 27B illustrates a cross-section of the surgical device 7070 shown in FIG. 27A. As shown in FIG. 27B, a suction tube 7005 extends from the suction outlet 7072 through the handle 7073 and the elongate member 7074 to the suction lumen 7004. The irrigation tube 7006 extends from the irrigation fluid inlet 7071 through the handle 7073 and the elongate member 7074 to the tip 7050. The interior space of the tip 7050 may be hollow, and the irrigation fluid may exit the irrigation lumens 7003 from the hollow interior space of the tip 7050. The irrigation tube 7006 may be the hollow space around or a tube surrounding the suction tube 7005 in the interior of the elongate member 7074 and the handle 7073. FIG. 27C is another illustration of the cross-section of the surgical device 7070 as shown in FIG. 27B. FIG. 27D shows another view of the surgical device 7070 as shown in FIG. 27A FIG. 27E shows a cross-section of the surgical device 7070 as shown in FIG. 27D. FIG. 28A is a closer view of the handle 7073 of the cross-section of the surgical device 7070 as shown in FIG. 27B. FIG. 28B is another illustration of the handle 7073 as shown in FIG. 28A. FIG. 29A is a closer view of the end effector 7000 of the surgical device 7070 as shown in FIG. 27D. FIG. 29B is a closer view of the end effector 7000 of the cross-section of the surgical device 7070 as shown in FIG. 27B. FIG. 29C is another illustration of the end effector 7000 as shown in FIG. 29B.

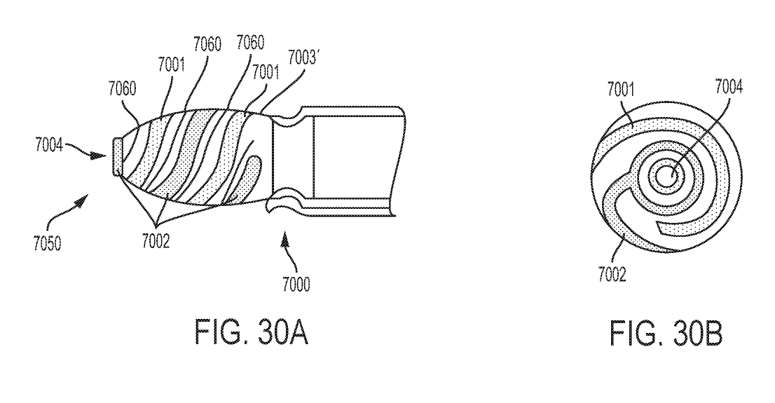

FIG. 30A depicts a closer view of one aspect of an end effector 7000 that may be part of medical device 7000. The bipolar electrodes 7001 and 7002 have a helical configuration following the contour of the tip 7050 of the end effector 7000. The helical electrodes 7001 and 7002 may be plated, wired, etc. Flow directing ridges 7060 may be provided on the tip 7050 between the electrodes 7001 and 7002 to guide the flow of an irrigation fluid. In some aspects, irrigation lumens 7003′ may be provided at the proximal end of the tip 7050. The suction lumen 7004 may be provided at the distal face of the tip 7050. FIG. 30B shows the tip 7050 of the end effector 7000 from the distal end of the end effector 7000.

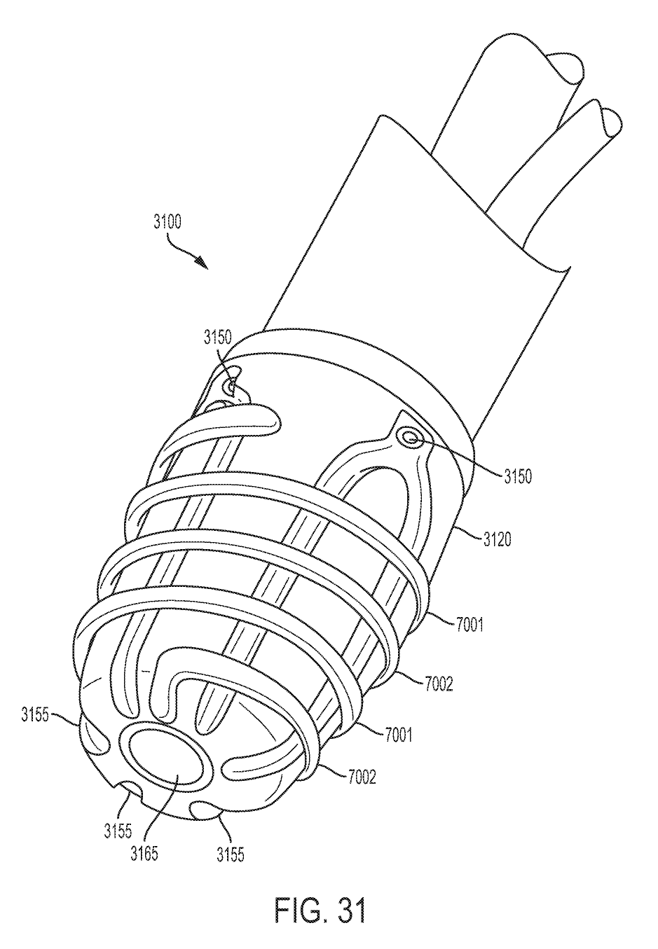

FIG. 31 depicts an alternative aspect of an end effector 3100 that incorporates helically wound electrodes similar to that of end effector 7000. The end effector 7000 includes flow directing ridges 7060 that may protrude from a surface of the end effector 7000. Such flow directing ridges 7060 may be configured to direct a flow of irrigation fluid across the surface of the end effector 7000.

End effector 3100 is composed of an end effector body 3120 having an exterior surface upon which are wound a positive electrode 7001 and a negative electrode 7002 in a helical manner. An evacuation port 3165 is disposed at the distal end of the end effector body 3120 and is configured to receive fluids including medical debris from the surgical site. Fluid discharge ports 3150 are disposed on the exterior surface of the end effector body 3120, for example at a proximal end. The fluid discharge ports 3150 are configured to deliver an irrigation fluid to the positive electrode 7001 and negative electrode 7002 as well as to the surgical site. While the irrigation fluid flow is directed by one or more flow directing ridges 7060 in the end effector 7000, the irrigation fluid flow is directed through a series of fluid flow channels 3155 that are disposed in the surface of the end effector body 3120. Such fluid flow channels 3155 are fluidically coupled to the fluid discharge ports 3150. As depicted in FIG. 31, such fluid flow channels 3155 are formed by a series of linear recesses that may be parallel to a longitudinal axis of the end effector body 3120.

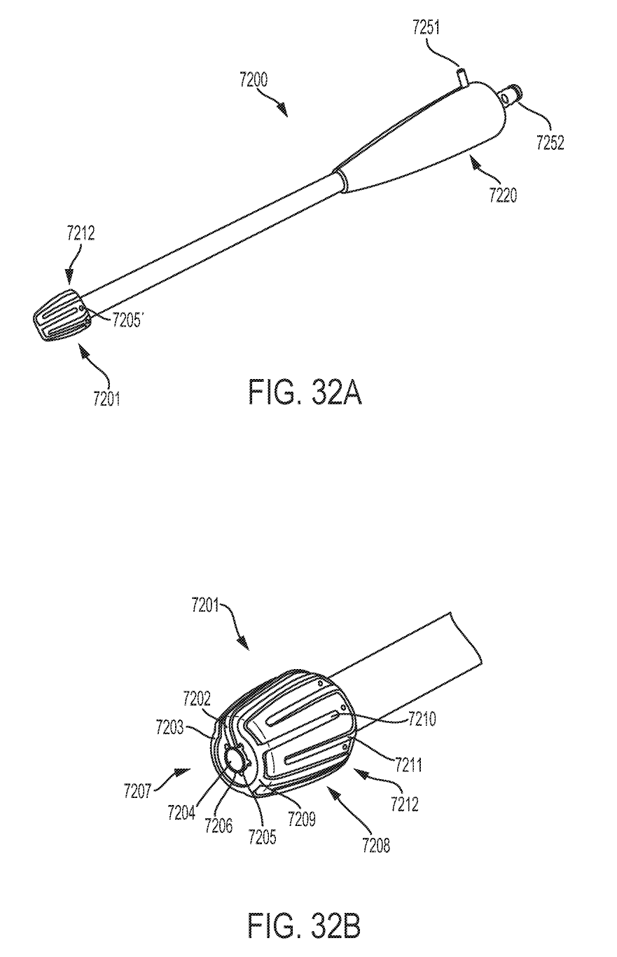

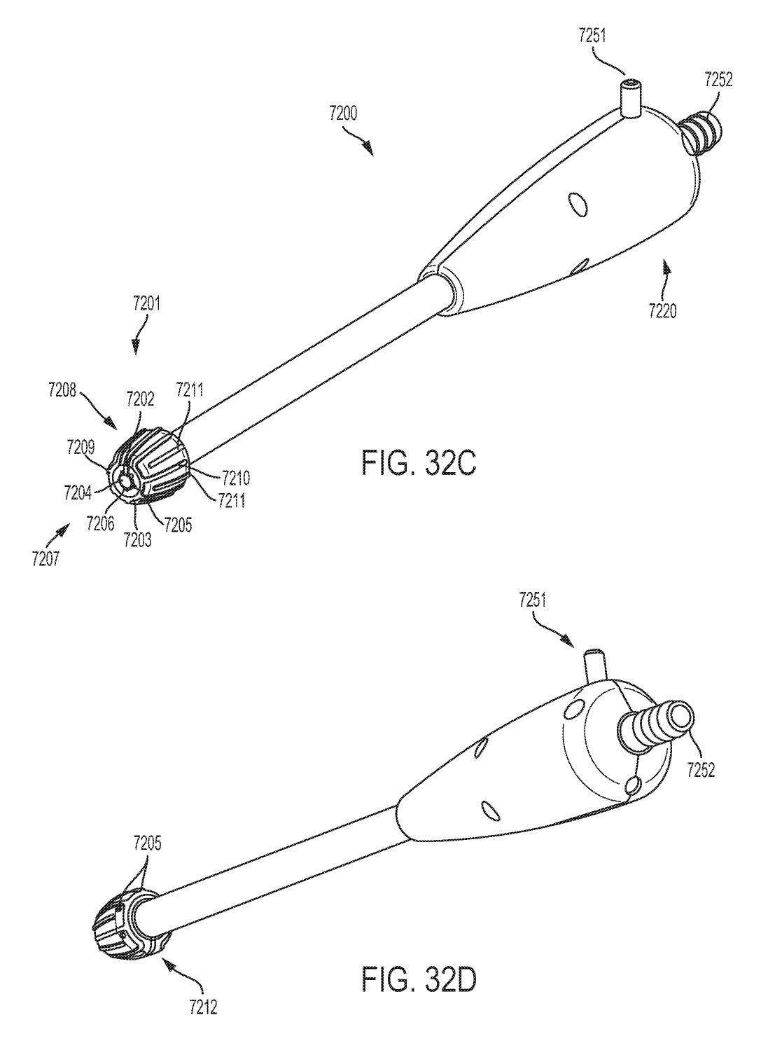

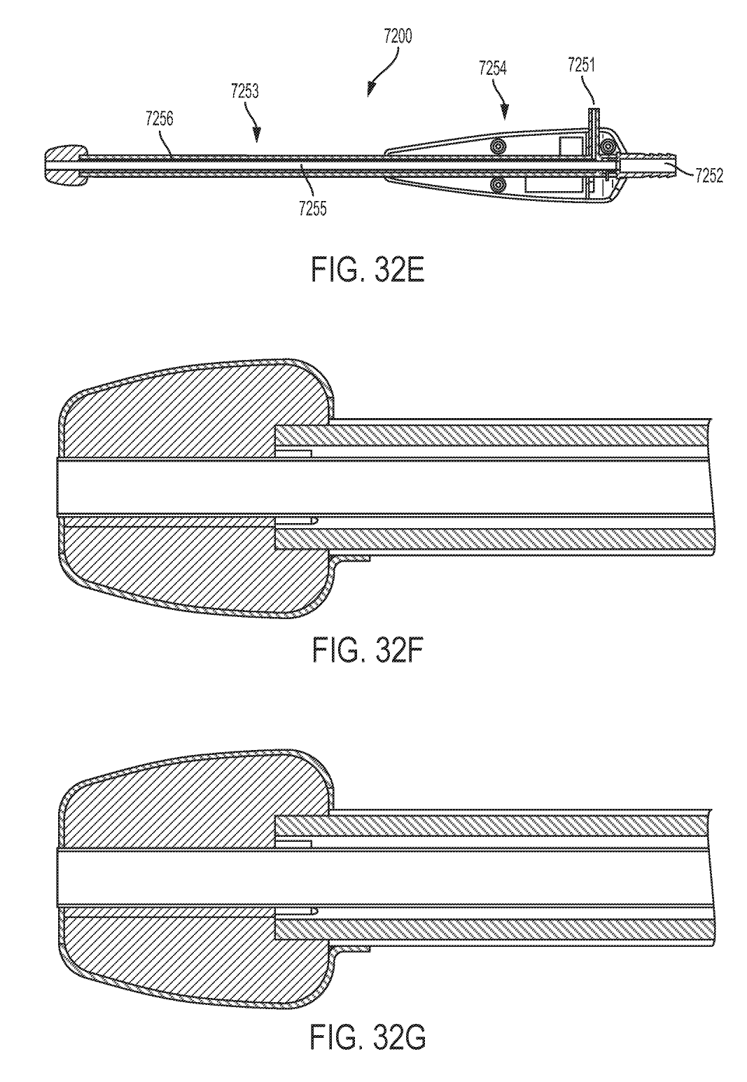

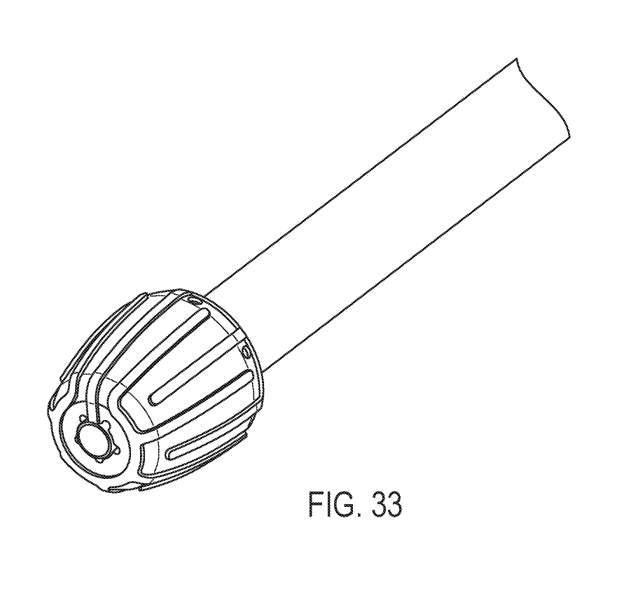

FIGS. 32A-G illustrate another aspect of an electrosurgical device composed of an end effector 7201 having an electrode configuration that differs from that depicted in FIGS. 26B, 30A,B, and 31. While FIGS. 26B, 30A,B, and 31 depict an electrosurgical device having an end effector composed of helically wound electrodes, FIGS. 32A-G depict an electrosurgical device having an end effector composed of interdigitated electrodes. The electrodes 7202 and 7203 may be plated or wired etc. A suction lumen 7204 is provided at a distal face 7207 of the end effector 7201 surrounded by a circle 7206 formed by the electrode 7202. The electrode 7202 extends from the circle 7206 from the distal face 7207 along the side surface 7208 of the end effector 7201. The electrode 7202 may include additional legs 7211 extending from the proximal end 7212 of the end effector 7201 in a distal direction. In some aspects, the legs 7211 may be linearly disposed on the outer surface of the end effector 7201 and may be parallel to a longitudinal axis of the end effector 7201. The other electrode 7203 of the opposite polarity forms an open circle 7209 with legs 7210 extending proximally from the open circle 7209 along the side surface 7208 of the end effector 7201. The electrode 7203 may include additional legs 7210 extending from the distal face 7207 of the end effector 7201 in a proximal direction. In some aspects, the legs 7210 may be linearly disposed on the outer surface of the end effector 7201 and may be parallel to a longitudinal axis of the end effector 7201. In one aspect, one or more irrigation lumens 7205 may be provided around the suction lumen 7204. FIG. 32A depicts a surgical device 7200 with the end effector 7201 as shown in FIG. 32B. In some aspects, one or more irrigation lumens 7205′ may also be provided at the proximal end 7212 of the end effector 7201. An irrigation fluid inlet 7251 and a suction outlet 7252 may be provided at the proximal end 7220 of the surgical device 7200. FIG. 32C is another illustration of the surgical device 7200 as shown in FIG. 32A. FIG. 32D is another view of the surgical device 7200 as illustrated in FIG. 32C. FIG. 33 is a closer view of the end effector 7201 of the surgical device 7200 as illustrated in FIG. 32C.

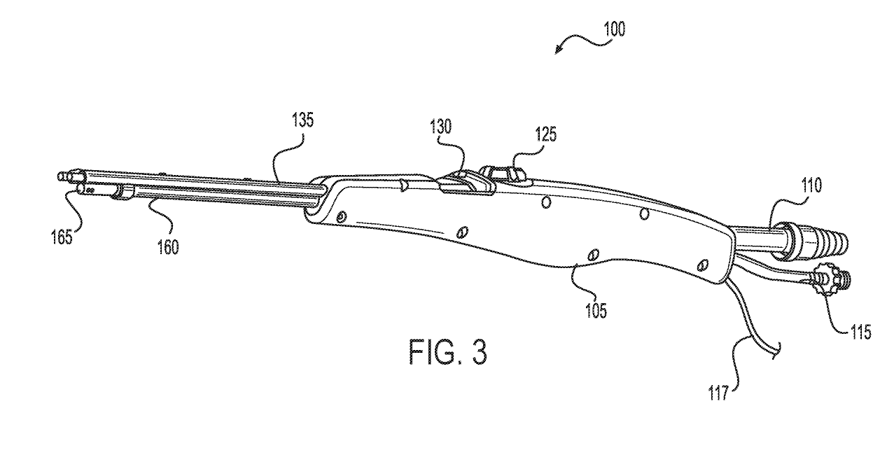

FIG. 32E shows a cross-section of the surgical device 7200 as illustrated in FIG. 32D. As shown in FIG. 32E, the surgical device 7200 includes an elongate member 7253, a handle 7254, the irrigation fluid inlet 7251, the suction outlet 7252, a suction tube 7255, and an irrigation tube 7256 similarly to those of the surgical device 7000 as shown in FIG. 27B. A difference between the surgical device 7000 as shown in FIG. 27B and the surgical device 7200 is the end effector 7201. FIG. 32F is a closer view of the cross-section of the end effector 7201 as shown in FIG. 3E. FIG. 32G is another illustration of the cross-section of the end effector 7201 as shown in FIG. 32F. FIG. 33 is a closer view of the end effector 7201 of the surgical device 7200 as illustrated in FIG. 32C.

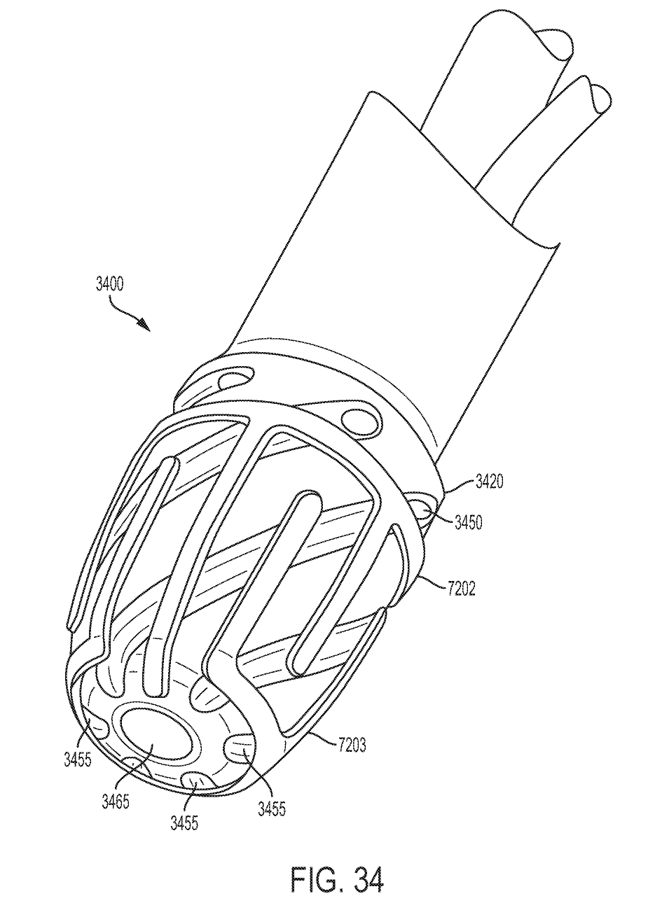

FIG. 34 depicts an alternative aspect of an end effector 3400 that incorporates interdigitated electrodes similar to that of end effector 7201. The end effector 7201 includes one or more irrigation lumens 7205 may be provided around the suction lumen 7204 disposed at the distal end of the end effector 7201.

End effector 3400 is composed of an end effector body 3420 having an exterior surface upon which are disposed interdigitating electrodes 7202 and 7203 in a manner similar to those depicted in FIG. 32B. An evacuation port 3465 is disposed at the distal end of the end effector body 3420 and is configured to receive fluids including medical debris from the surgical site. Fluid discharge ports 3450 are disposed on the exterior surface of the end effector body 3420, for example at a proximal end. The fluid discharge ports 3450 are configured to deliver an irrigation fluid to the interdigitating electrodes 7202 and 7203 as well as to the surgical site. Similar to the aspect of end effector 3100, irrigation fluid flow is directed through a series of fluid flow channels 3455 that are disposed in the surface of the end effector body 3420. Such fluid flow channels 3455 are fluidically coupled to the fluid discharge ports 3450. As depicted in FIG. 34, such fluid flow channels 3455 are formed by a series of helically fabricated recesses in the surface of the end effector body 3120 and may have helical axes parallel to a longitudinal axis of the end effector body 3420.

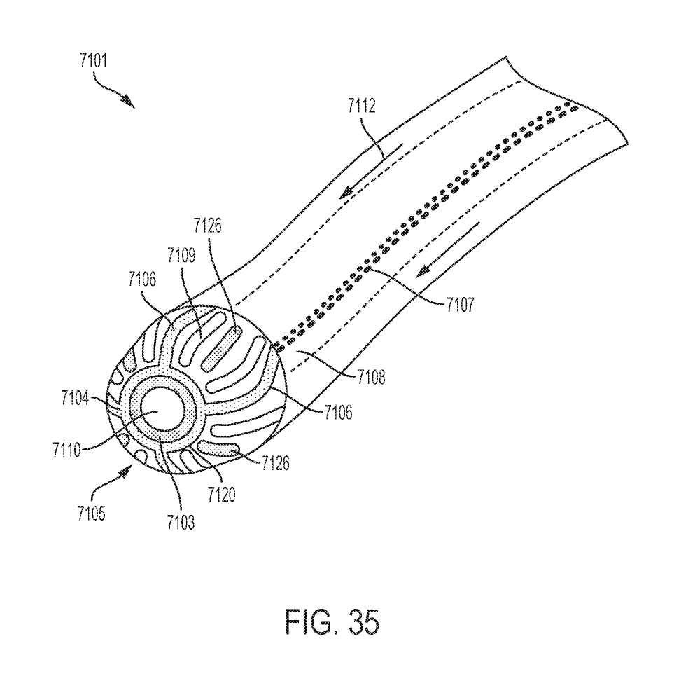

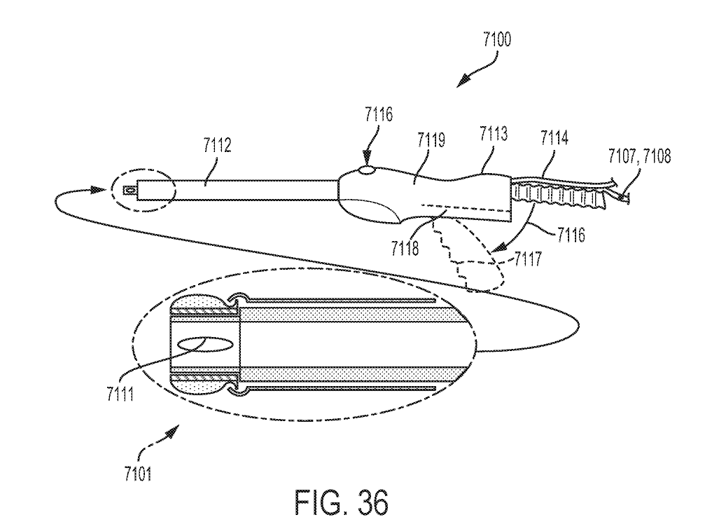

Referring to FIG. 35, another embodiment of the end effector 7101 is illustrated. The end effector 7101 can be constructed from insulating materials and electrodes. The insulating materials may be ceramics, polymers, elastomers, etc. The end effector 7101 can be overmolded, plated, etc. The bipolar electrodes 7120 and 7102 each include a circle 7103/7104 on the distal face 7105 of the end effector 7101. The bipolar electrodes 7120 and 7102 can be double helix, plated traces, wires, sheet metal, flex circuit, etc. Electrode legs 7106 extend from the circle 7104. The electrode legs 7106 and electrode legs of 7126 the opposite polarity to the power cables 7107 and 7108 supplying energy of opposite polarities that extend through the elongate member 7112 of the surgical device 7100 as shown in FIG. 36. The elongate member 7112 may include a sheath. The elongate member 7112 has an electrically insulating exterior and contains in its interior wires, ribbons, or plated traces, such as the power cables 7107 and 7108. The insulating exterior of the elongate member 7112 may be a sheath. The sheath can be encapsulated--both the inner diameter and the outer diameter of the sheath have electrical insulation. The portion between the inner diameter and the outer diameter may be conductive, and the conductive part may be connected to one of the electrodes 7120 and 7102. The sheath can be produced by co-extrusion or film encapsulation processes.

Irrigation channels 7109 may be provided on the side surface of the end effector 7101. The irrigation channels 7109 may also guide the flow of the irrigation fluid exiting the irrigation channels 7109. The end effector may include other flow directing features, such as ridges or bumps. The suction lumen 7110 may be provided at the distal face of the end effector 7101 inside the electrode circles 7103 and 7104. The fluid circuit is formed between the irrigation channels 7109 and the suction lumen 7110. The suction tube may be an inner tube in the center of the elongate member 7112. The inner tube may be made of a rigid metal, such as Al, etc. The inner tube may also be malleable. The inner tube may have an exterior surface coated or insulated such as with shrink tube, parylene, etc. The inner tube may be connected to the other electrode with the opposite polarity to the electrode that is connected to the conductive portion of the sheath. The inner diameter of the inner tube may be used as the suction tube. The irrigation tube may be an annulus between the inner tube and the sheath.

FIG. 36 includes a cross-section view of the end effector 7101 as shown in FIG. 35. As shown in FIG. 36, side suction lumen slots 7111 may also be provided at a side surface of the end effector 7101. FIG. 36 also illustrates the elongate member 7112 and the handle 7113 of the surgical device 7100. As shown by the line with an arrow, the end effector is provided at the distal end of the elongate member 7112. The elongate member 7112 extends from the end effector 7101 at the distal end to the handle 7113 at the proximal end. From the proximal end of the handle 7113, the irrigation tube 7114, the power cables 7107 and 7108, and the suction tube 7115 extend to their respective devices exterior to the surgical device 7100. The handle 7113 may include an activation switch 7116 that activates the power and irrigation. The switch 7116 may have a first position that may turn on a pump that supplies the irrigation fluid to the end effector 7101. The switch 7116 may be mechanically or electrically tied to a valve that allows a pressurized reservoir to empty and therefore, supply the irrigation fluid to the end effector. Alternatively, an in-line valve, separate from the handle, fitting with an ON/OFF or graduated control, e.g., a stop lock lever, may be provided to control the irrigation. This in-line valve or a valve/fitting on handle may provide in-field ON/OFF control of the irrigation. An irrigation button and a suction button may also be provided on the handle 7113 and are not shown here. The handle 7113 may also include a grip 7117 as shown by the dotted lines extending from the body 7119 of the handle 7113. The handle 7113 may also include a slot 7118 as shown by the dotted line in the body 7119 of the handle 7113, and the irrigation tube 7114, the power cables 7107 and 7108, and the suction tube 7115 may be pulled out from the slot 7118.

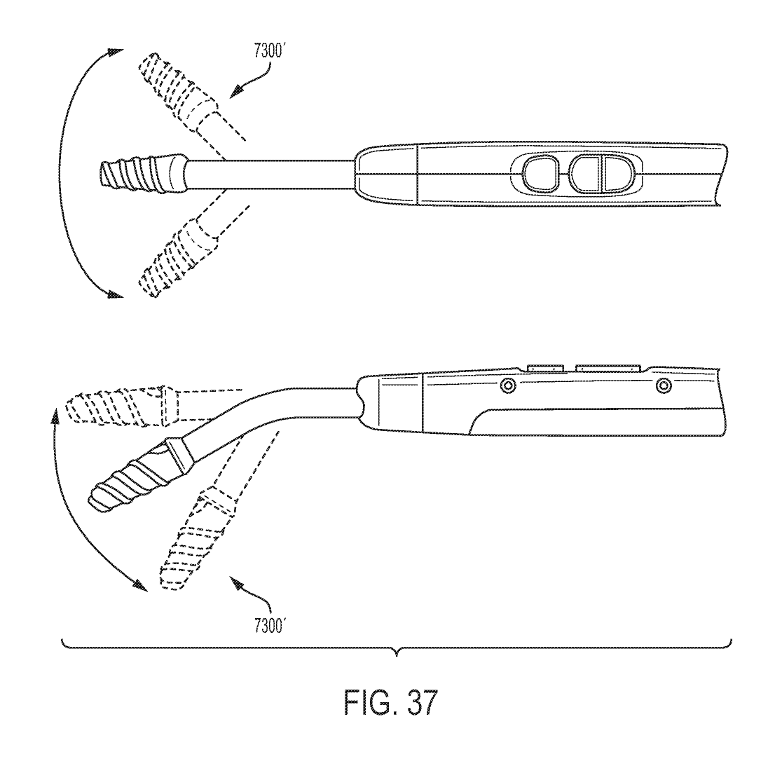

FIG. 37 depicts an end effector 7300′ having a flexible neck 7312′. The neck 7312′ may be a bendable spring tube. As depicted in FIG. 37, the neck 7312′ may be bent in any direction. It may be understood that such an end effector may be placed at a distal end of an end effector neck or of a shaft of an electrosurgical device. In some aspects, the end effector neck or device shaft may be flexible. Thus, although FIG. 35 is more specifically directed to depicting end effector 7101, it may be observed that the elongate member 7112 (or shaft) associated with the end effector 7101 is bendable or flexible. While FIGS. 35 and 37 are directed to specific examples of a flexible elongate member (or shaft) or of an end effector neck, it may be recognized that a shaft portion of an electrosurgical device having any configuration of end effector may exhibit a flexible aspect. For example, and without limitation, electrosurgical devices depicted in any one or more of FIGS. 26-36, may include a shaft, shaft portion, elongate member, or neck that is flexible. In some aspects, an electrosurgical device may have a flexible shaft, shaft portion, elongate member, or neck configured to bend in response to the end effector being placed proximate to a solid tissue, such as muscle or bone. Such a flexible shaft, shaft portion, elongate member, or neck may be configured to return to an elongated and unbent or un-flexed state once the end effector no longer contacts the solid tissue. In an alternative aspect, the shaft, shaft portion, elongate member, or neck may be configured so that a user is required to apply a manual force to the shaft, shaft portion, elongate member, or neck thereby imparting curvature to the shaft, shaft portion, elongate member, or neck. Once the curvature has been manually applied to the shaft, shaft portion, elongate member, or neck, the shaft, shaft portion, elongate member, or neck may retain the curvature throughout use until the user applies an opposing force to the shaft, shaft portion, elongate member, or neck thereby restoring the shaft, shaft portion, elongate member, or neck to its original elongated and unbent or un-flexed state.

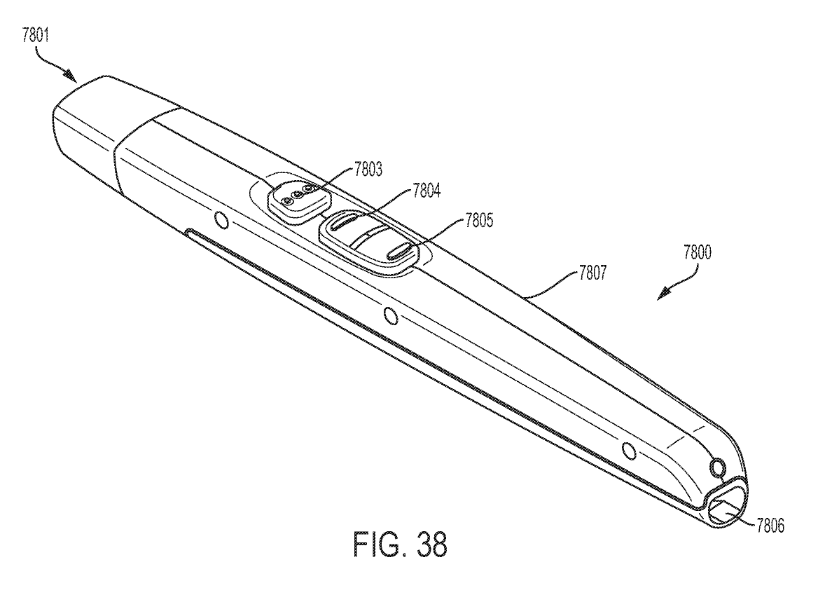

FIGS. 38-43 depict a variety of modular medical devices which may be composed of components that can be readily assembled and/or disassembled by a user to suit a variety of surgical requirements. For example, such components may be readily replaced if damaged, or discarded to reduce the possibility of cross-contamination between patients. FIGS. 44-62 depict a variety of end effectors that may be used with such modular medical devices.

Referring now to FIG. 38, an aspect of a modular handle 7800 is illustrated. The handle 7800 has a distal end 7801 where a modular probe can be installed. The handle 7800 also has a proximal end 7802 where an outlet 7806 is provided. The irrigation and suction tubes can be connected to the handle 7800 though the outlet 7806. On a surface 7807 of the handle 7800, a power button 7803, an irrigation button 7804, and a suction button 7805 may be provided. The buttons may function as described above. Modular end effectors and shaft assemblies or modular probes, depicted in FIGS. 44-61, can be connected to the handle 7800.

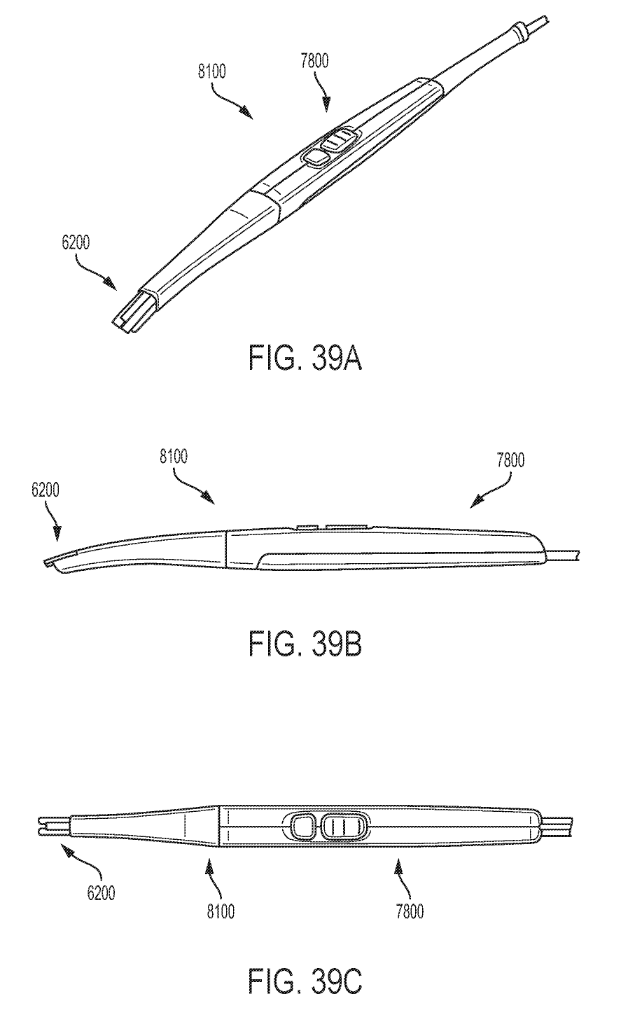

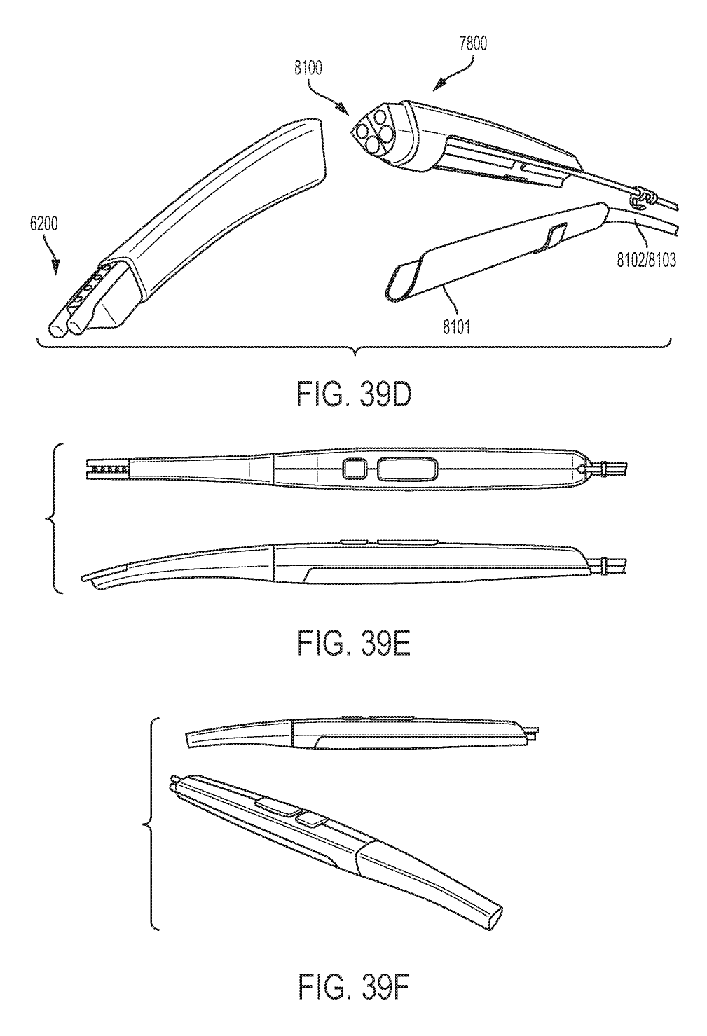

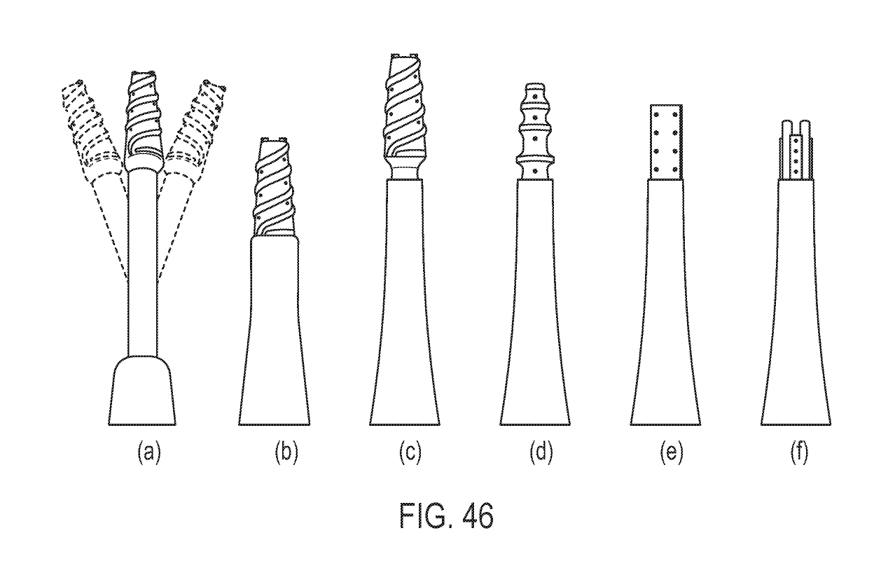

FIGS. 39A-F illustrate an aspect of a modular surgical device 8100 assembled by the handle 7800 as shown in FIG. 38 and along with an exemplary modular probe (f) in FIG. 46 with the end effector 6200. As shown in FIGS. 39B,C, in some embodiments, the handle 7800 may have a longitudinal length of about 170 mm, a thickness of about 23 mm, and a width of about 22.5 mm. FIG. 39D illustrates the surgical device 8100 when it is dissembled into pieces, including the handle 7800, which is reusable, the interchangeable end effector 6200, and a disposable tubing cartridge 8101 that includes the suction and irrigation tubes 8102 and 8103. As shown in FIG. 39D, the handle 7800 includes an interface 8104 configured to connect the handle 7800 to the end effector 6200. FIG. 39E is another illustration of the surgical device 8100 as shown in FIGS. 39A-C. A difference is the buttons may have a different color and/or design. FIG. 39F shows two other views of the surgical device 8100 as shown in FIG. 39E.

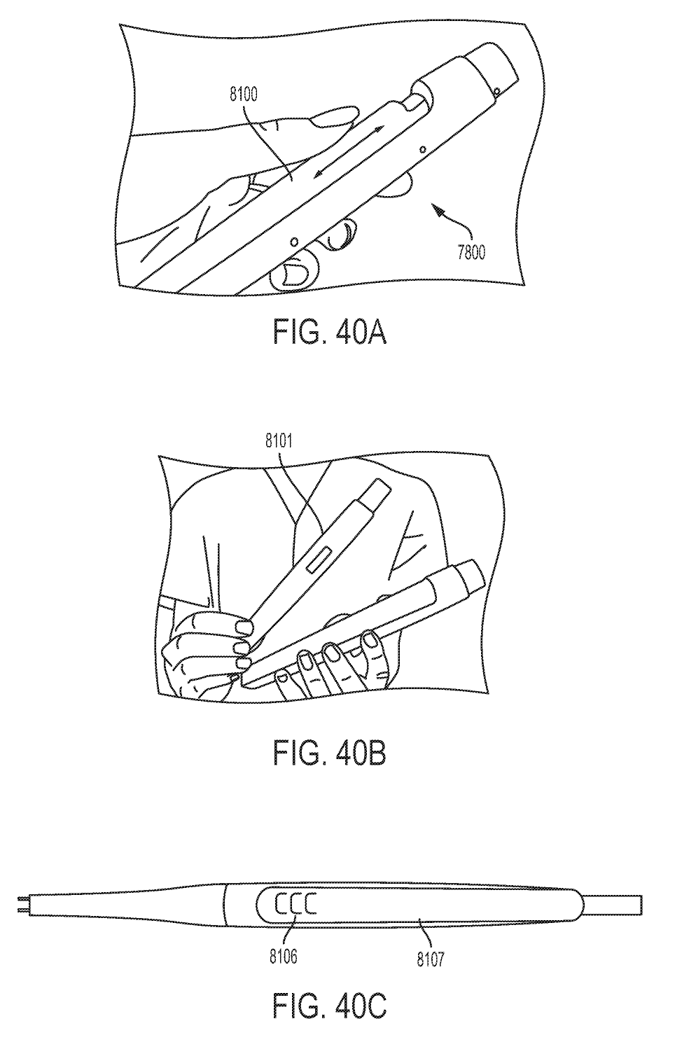

FIGS. 40A,B depict that the tubing cartridge 8101 may be dissembled from and assembled onto the handle 7800 by sliding the cartridge 8101 along a longitudinal direction of the handle 7800. FIG. 40C is another illustration of the surgical device 8100 showing a bottom side view of the device 8100. As shown here, a plurality of ribs 8106 may be provided on an exterior surface 8107 of the tubing cartridge 8101. The ribs 8106 provide more friction at the surface 8107 so that the user can slide the cartridge 8101 onto and off the handle 7800 more easily.

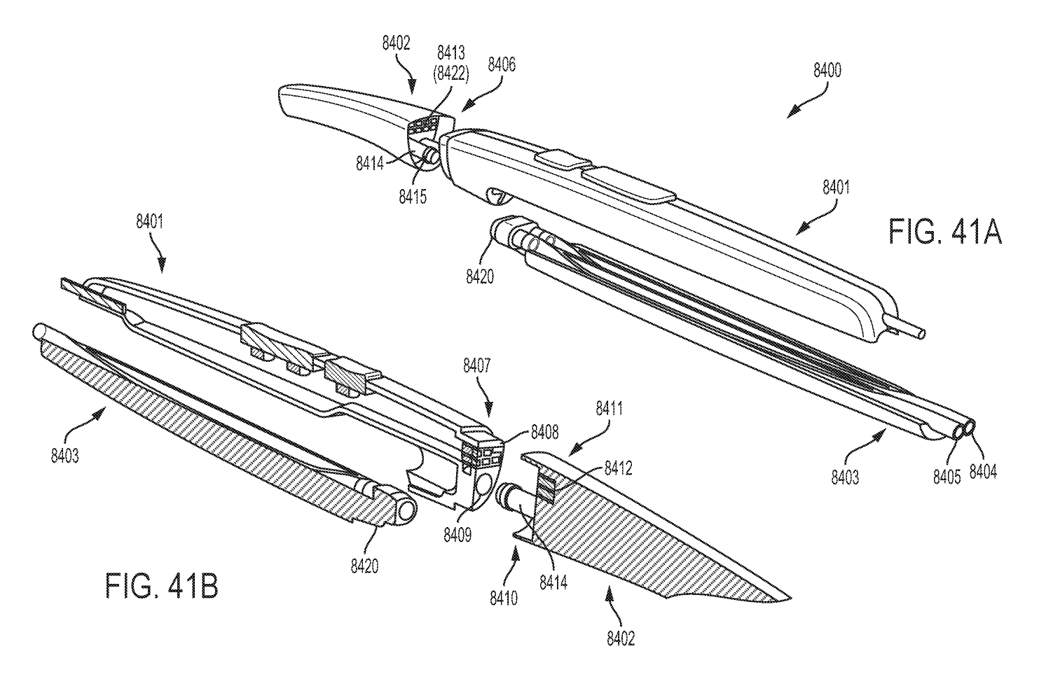

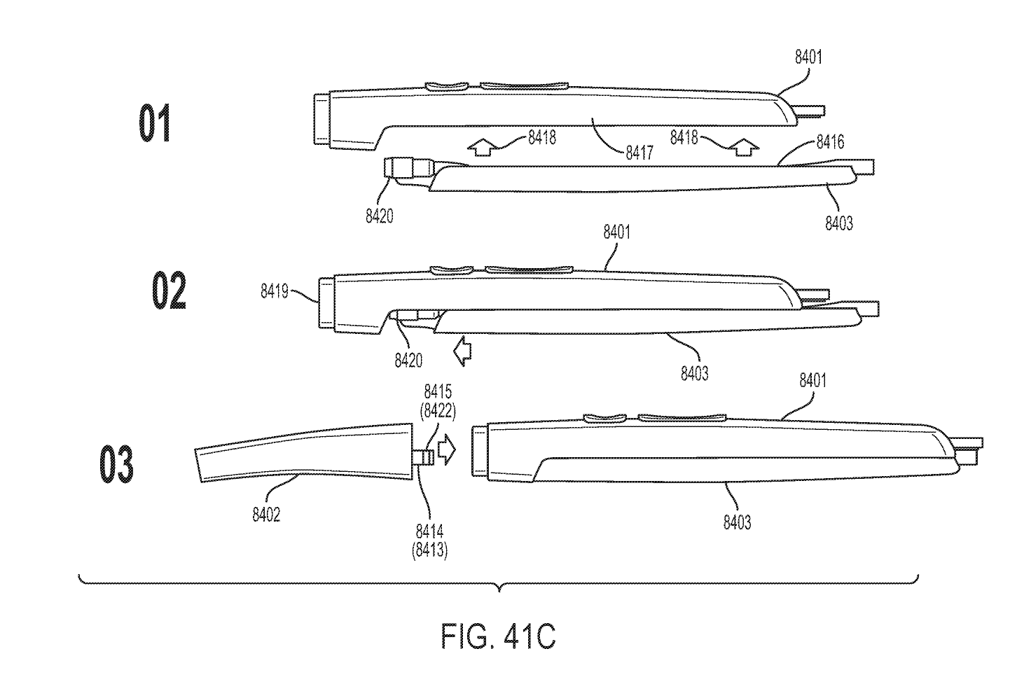

Another modular medical device is depicted in FIGS. 41A-C. FIG. 41A illustrates a surgical device assembly 8400. FIG. 41B shows a cross-section view of the surgical device assembly 8400 from another angle. It is noted that only basic structures of the assembly 8400 are shown in FIG. 41B to illustrate how the assembly 8400 are assembled. The detailed inner structures, such as the electric circuits and connections, are not shown here. As shown in FIGS. 41-C, the assembly 8400 may includes the handle 8401, the end effector and shaft assembly 8402, and the lumen cartridge 8403. The irrigation lumen 8404 and the suction lumen 8405 are fixated at the bottom of the lumen cartridge 8403. The handle 8401 includes an interface 8407 at the distal end 8406 of the handle 8401, and the interface 8407 may include connectors 8408 and an aperture 8409 for lumen connection. The connectors 8408 may be electrically conductive. The lumen cartridge 8403 may be disconnectably installed at the bottom of the handle 8401. The end effector and shaft assembly 8402 has an interface 8410 at its proximal end 8411. This interface 8410 corresponds to and connects the interface 8407 of the handle 8401. The interface 8410 may include connectors 8412 corresponding to the connectors 8408 on the interface 8407 of the handle 8401. The irrigation lumen 8413 and the suction lumen 8414 in the end effector and shaft assembly 8402 each have a lumen connection, such as the suction lumen connection 8415 and the irrigation lumen connection 8422, which connects the corresponding lumen in the lumen cartridge 8402 when the cartridge 8402 is installed in the handle 8401. Only one lumen 8414 is shown in FIG. 41B since this figure shows a cross-section view, and therefore, only half of the device assembly 8400 is illustrated. It is understood that the other lumen 8413 is included in the other half of the device assembly 8400 not shown in FIG. 41B.

The handle 8401, the end effector and shaft assembly 8402, and the lumen cartridge 8403 may be assembled as shown in FIG. 41C. In Step 01, the longitudinal edges 8416 of the lumen cartridge 8403 may be fitted to the longitudinal edges 8417 at the bottom of the handle 8401 in the direction shown by the arrows 8418 to form an assembly 8419 of the handle 8401 and the cartridge 8403 as shown in Step 02. Then, the lumen cartridge 8403 may be slid along the longitudinal edges 8417 towards the distal end 8406 of the handle 8401 until the lumen connection 8420 of the cartridge 8403 reaches an inner surface 8421 of the aperture 8409, and the cartridge 8403 is securely fitted and connected to the handle 8401. Then in Step 03, the end effector and shaft assembly 8402 may be assembled to the handle and cartridge assembly 8419 by fitting the suction lumen connection 8415 and the irrigation lumen connection 8422 into the aperture 8409 and then into the lumen connection 8420 of the cartridge 8403

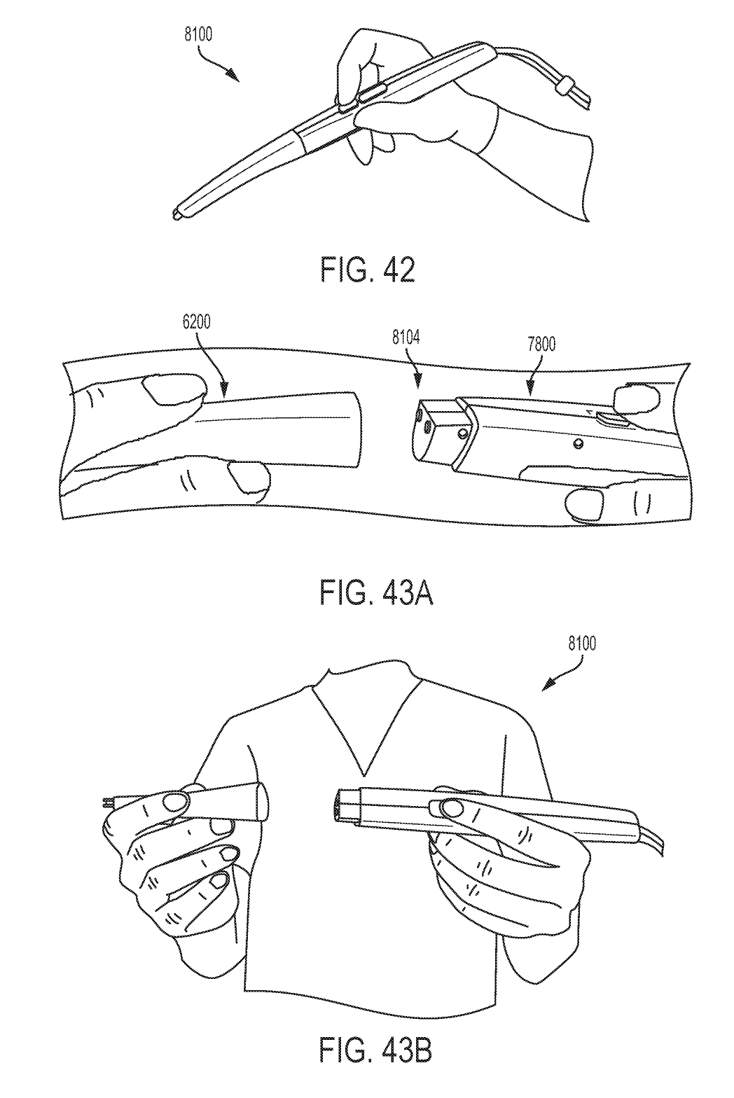

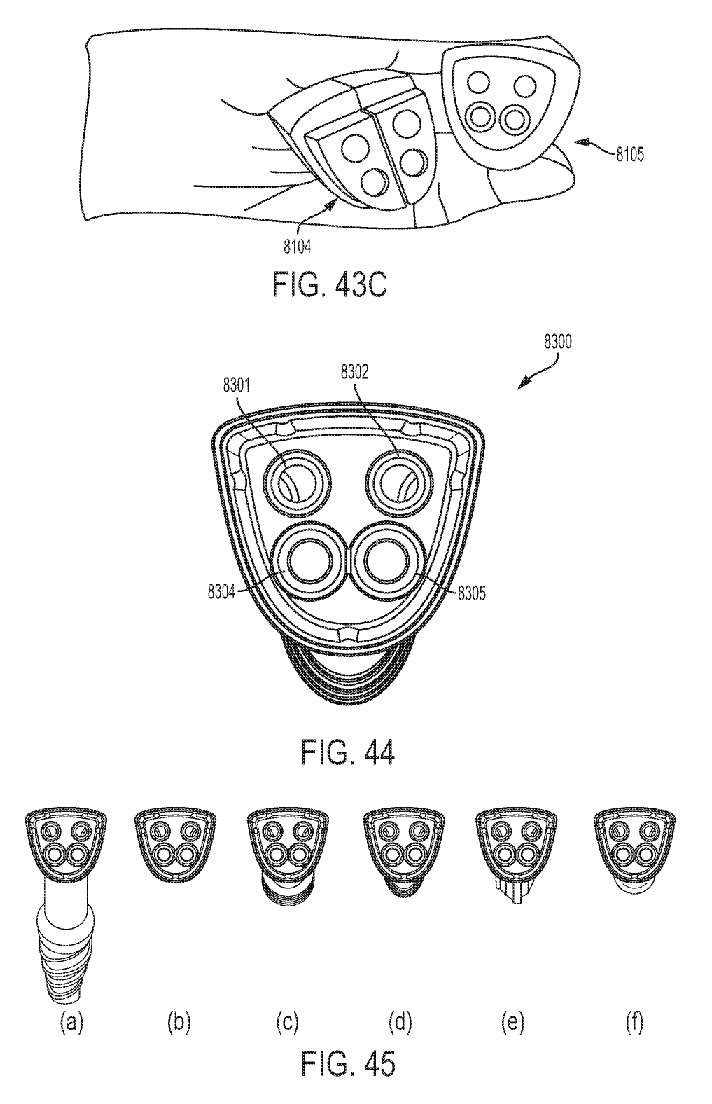

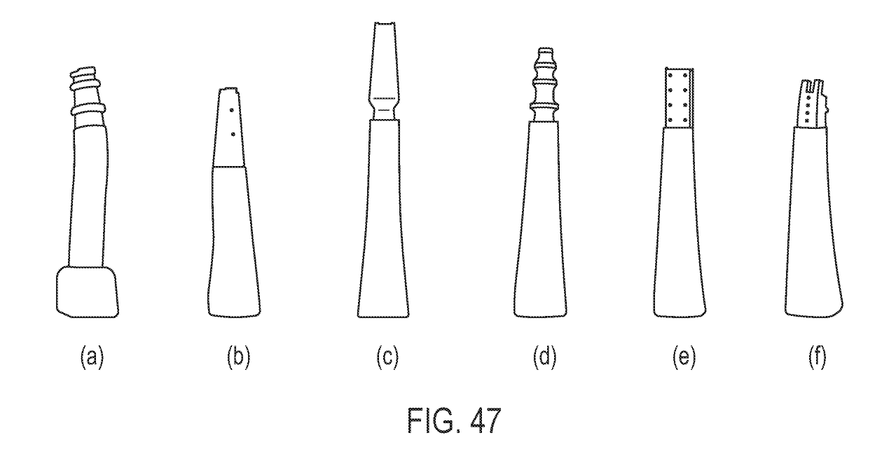

FIG. 42 is another illustration of the surgical device 8100 held by a hand of the user. FIG. 43A illustrates that the device 8100 can be dissembled between the end effector 6200 and the handle 7800 at the handle interface 8104. FIG. 43B is farther view of FIG. 43A. FIG. 43C shows the handle interface 8104 and the end effector interface 8105. As shown here, two magnets are used on the two interfaces 8104 and 8105 for connection thereof. An example interface 8300 is shown in FIG. 44. This interface 8300 connects to the interface 8104 of the handle as shown in FIG. 39D. The interface 8300 includes two conductors 8301 and 8302, a suction lumen hole 8304, and an irrigation lumen hole 8305. Snap o-rings may be provided at the two holes 8304 and 8305 to secure the suction tube and irrigation tube. The modular probe, i.e., the end effector and shaft assembly, with this interface 8300 may be held to the handle, which may be cleanable/sterilizable and reused, by way of magnets, a latch, a friction fit, etc. If magnets are used for retention of the end effector and shaft assembly to the handle, these magnets may be placed at the conductors 8301 and 8302 and may be used, at least in part, to carry electrical energy from conductors in the handle to the tissue effecting electrodes or other electrical components such as, for example, an identification/authentication means such as an electronic storage device, e.g. EEPROM, ASIC, general electric circuit, etc. [00254] Referring to FIG. 45, the example modular probes (a)-(f) as shown, for example, in FIGS. 46 and 47, are illustrated from the proximal ends. As shown here, each probe has an interface at the proximal end.

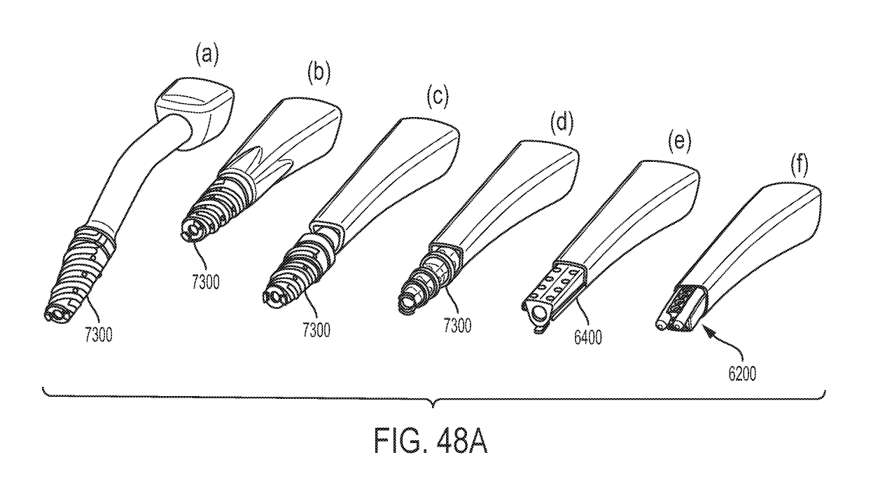

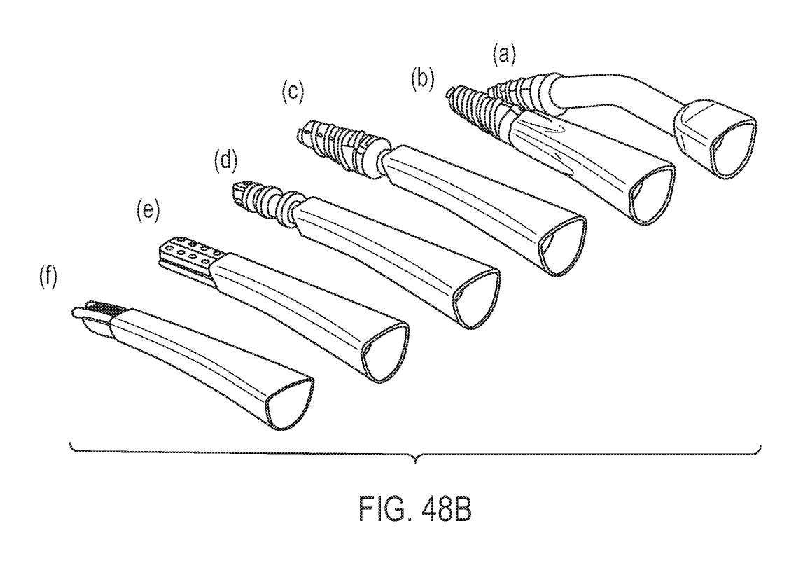

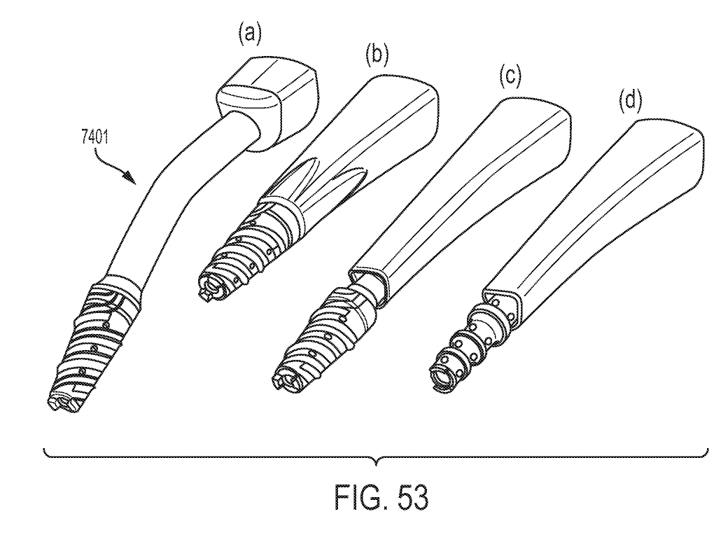

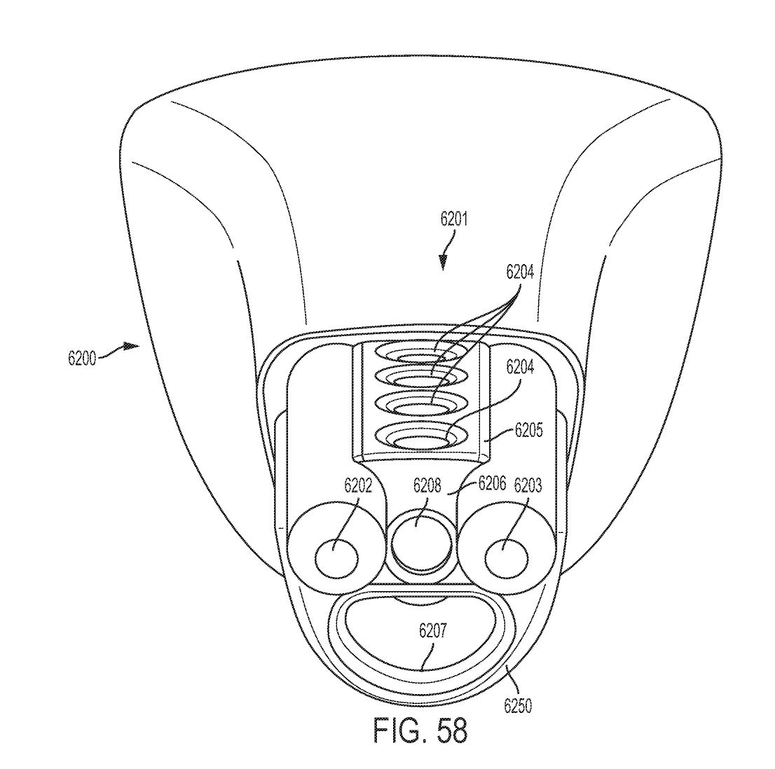

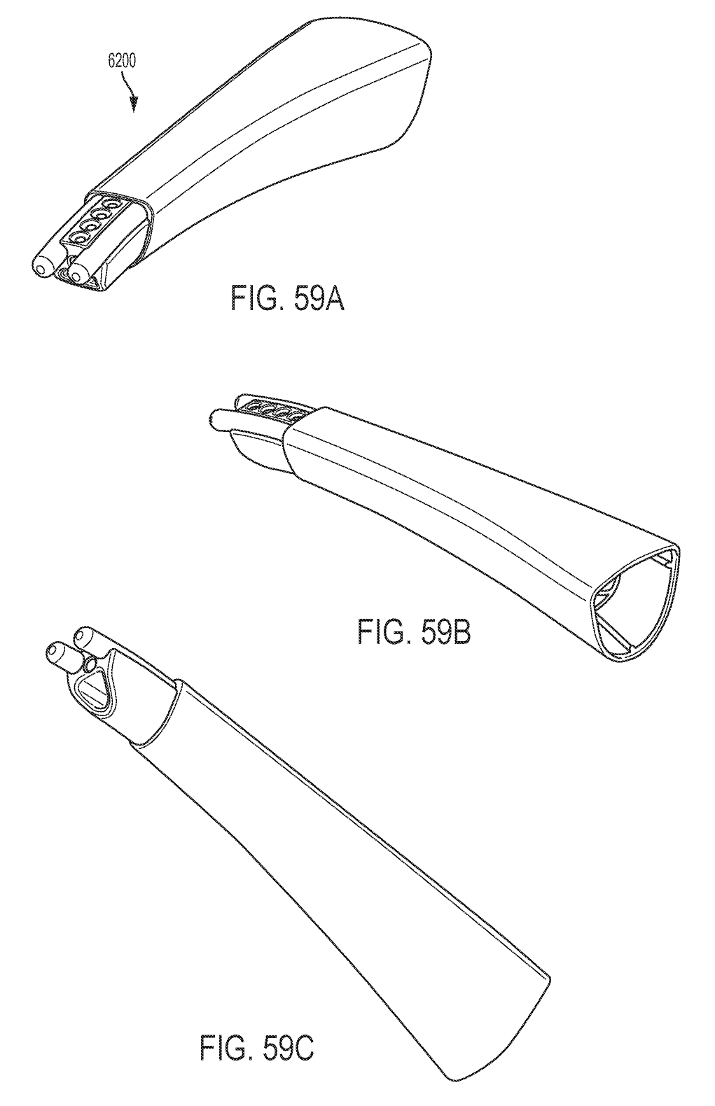

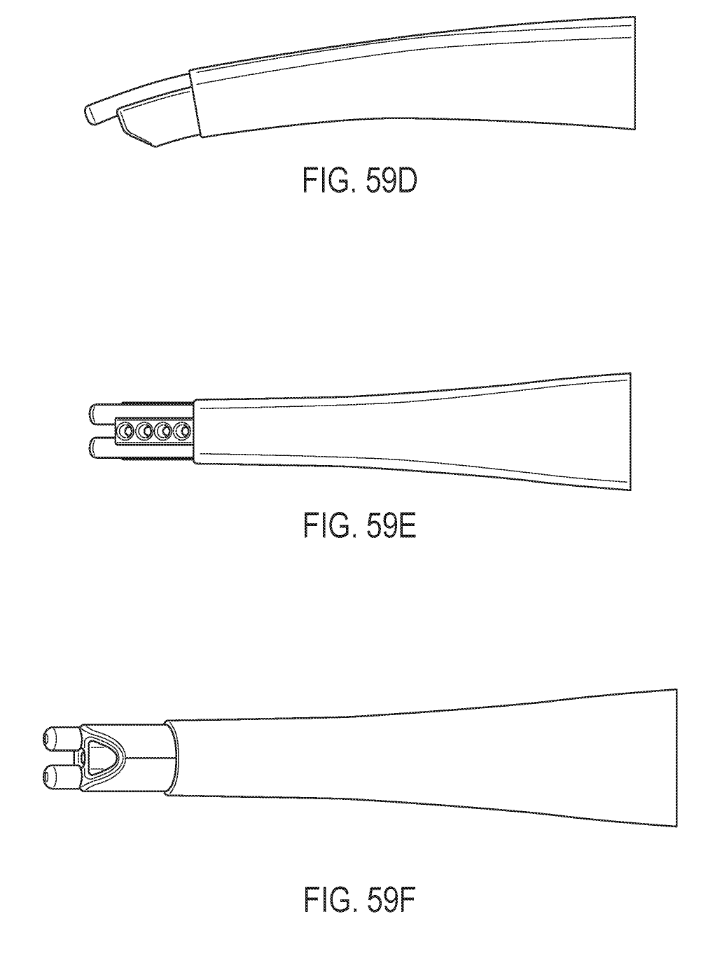

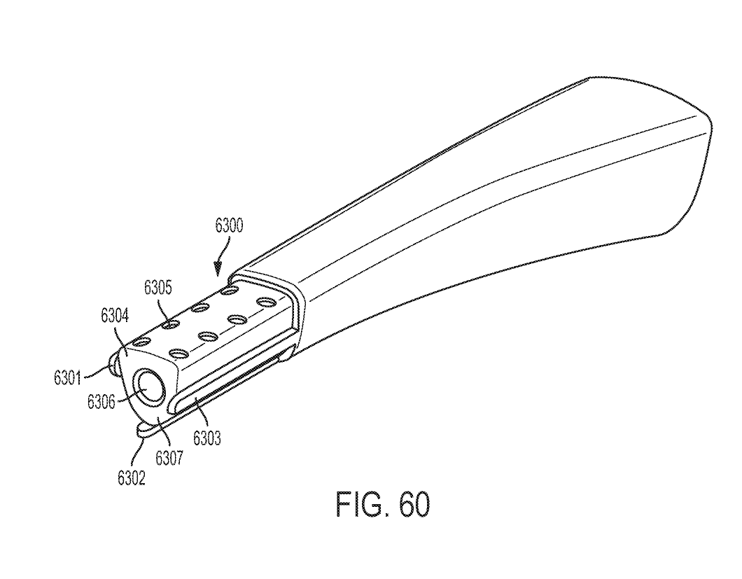

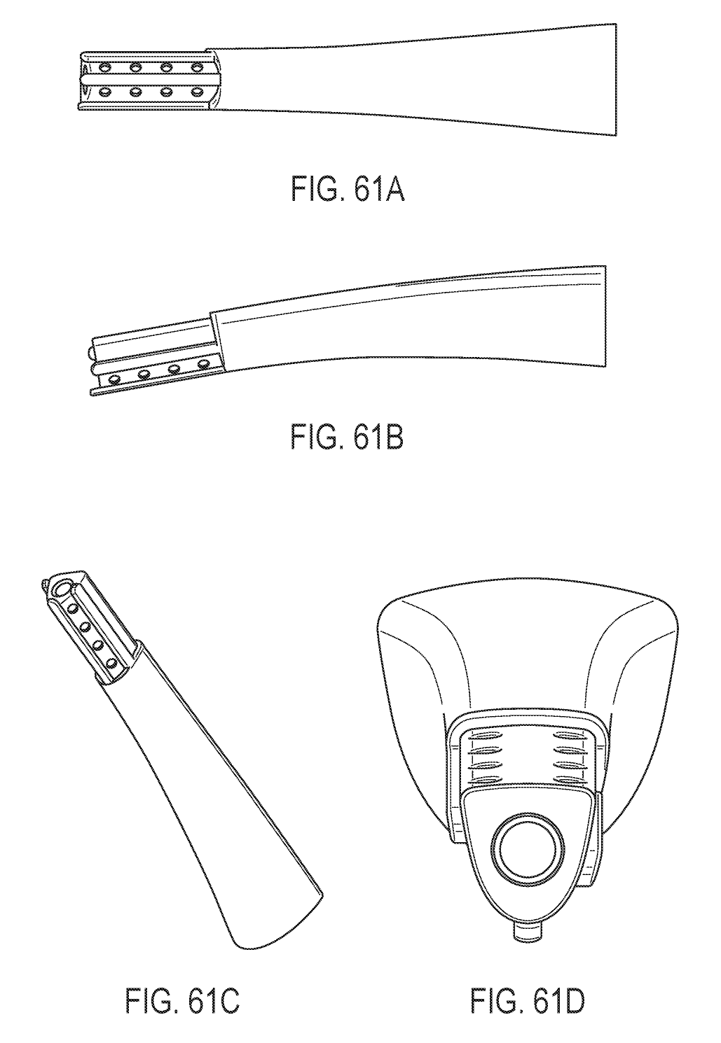

FIGS. 46-61 depict a number of examples of modular probes. For example, FIG. 46 is an illustration of aspects of modular probes (a)-(f) further depicted in FIG. 48A. FIG. 47 is another illustration of the example modular probes (a)-(f) as shown in FIG. 46. [00250] FIGS. 48A,B depict some aspects of end effectors and shaft assemblies or modular probes (a)-(f). The probes (b)-(d) are also shown among those depicted in FIG. 53, (a)-(c), respectively. The probe (e) has the end effector 6400. Various views of probe (e) are further depicted in FIGS. 60 and 61. The probe (f) has the end effector 6200. Various views of probe (f) are further depicted in FIGS. 58 and 59.

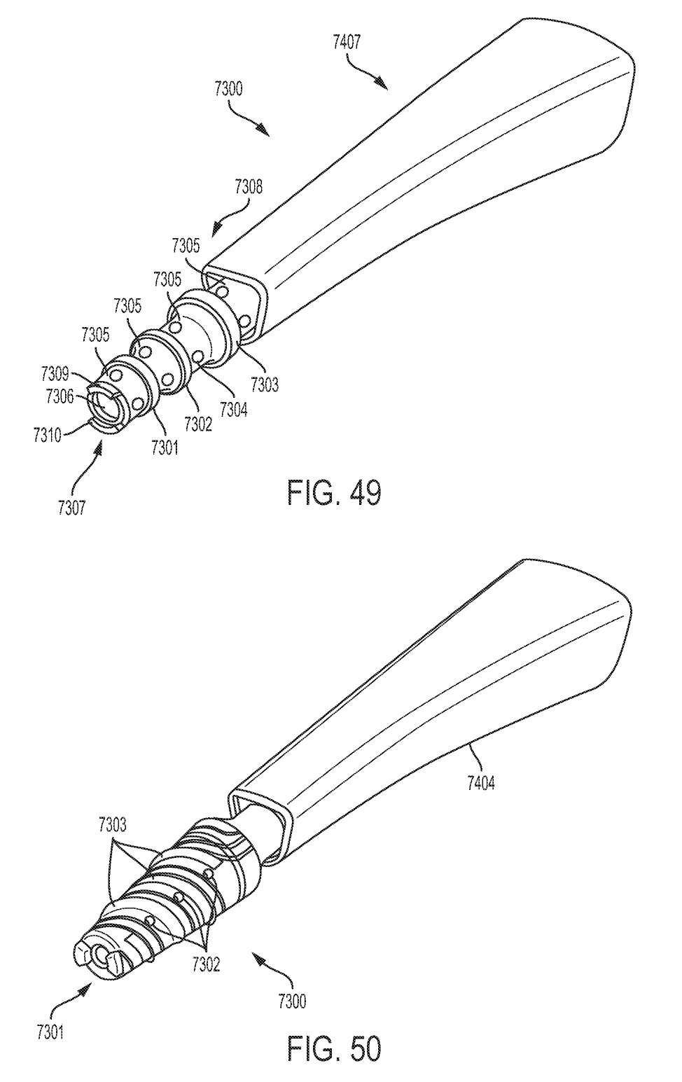

FIG. 49 shows another example embodiment of the end effector 7300 with circular electrodes 7301-7303. Irrigation fluid outlets 7304 are provided on the surfaces 7305 between the electrodes 7301-7303 and between the electrode 7203 and the proximal end 7308 of the end effector 7300. Every two of the electrodes 7301-7303 next to each other may have the same or opposite polarities. The suction lumen 7306 is provided at the distal face 7307 of the end effector 7300. The two tips 7309 and 7310 at the distal face 7307 at the edge of the suction lumen 7306 may be conducting and have two opposite polarities, so that coagulation can be performed between the two tips 7309 and 7310 at the distal face 7307 of the end effector 7300.

FIG. 50 illustrates another example embodiment of the spiral end effector 7300′ with helical electrodes 7201 and 7202. A difference between the end effector 7300′ as shown here and the end effector 7000 as shown in FIGS. 30A,B is that the distal face 7301′ of the end effector 7300′ has a configuration similar to the distal face 7207 of the end effector 7200 as shown in FIGS. 32A,B. Another difference is the irrigation lumen 7302′ is provided on the surfaces 7303′ between the helixes of the electrodes 7201 and 7202.

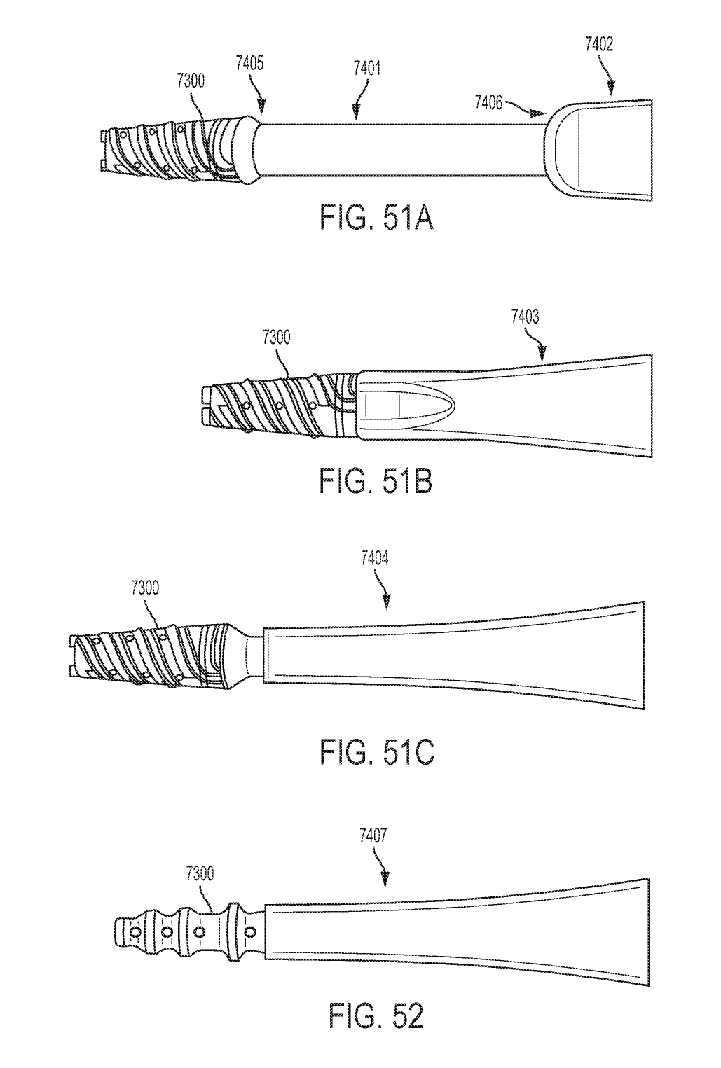

FIGS. 51A-C and 52 illustrates different embodiments of the end effector with the elongate member of the surgical device. In FIG. 51A, the end effector 7300′ is connected to a shaft 7401 at the distal end 7405 of the shaft 7401. The proximal end 7406 of the shaft 7401 is connected to an interface body 7402 used to connect to the handle of the surgical device. The elongate member 7403 in FIG. 51B has a different configuration from and is shorter than that of the elongate member 7404 in FIG. 51C. FIG. 52 has an elongate member 7407, but has the end effector 7300 as shown in FIG. 51A. FIG. 51C is yet another view of the end effector 7300′, and FIG. 52 is another view of the end effector 7300. FIGS. 53A-D are views of the end effectors with the elongate members as shown in FIGS. 51A-C and 52, respectively, from another angle. As shown in FIG. 53A, the shaft 7401 may be bendable.

As illustrated in FIGS. 51A-C, 52, and 53, the end effector with at least a portion of the elongate member can be separated from the handle or the other portion of the elongate member. Modular suction/irrigation/bipolar probes including different end effectors, such as the ones shown in FIGS. 51A-C, 52, and 53, may be provided separately depending on the surgeries and/or preference of the user.

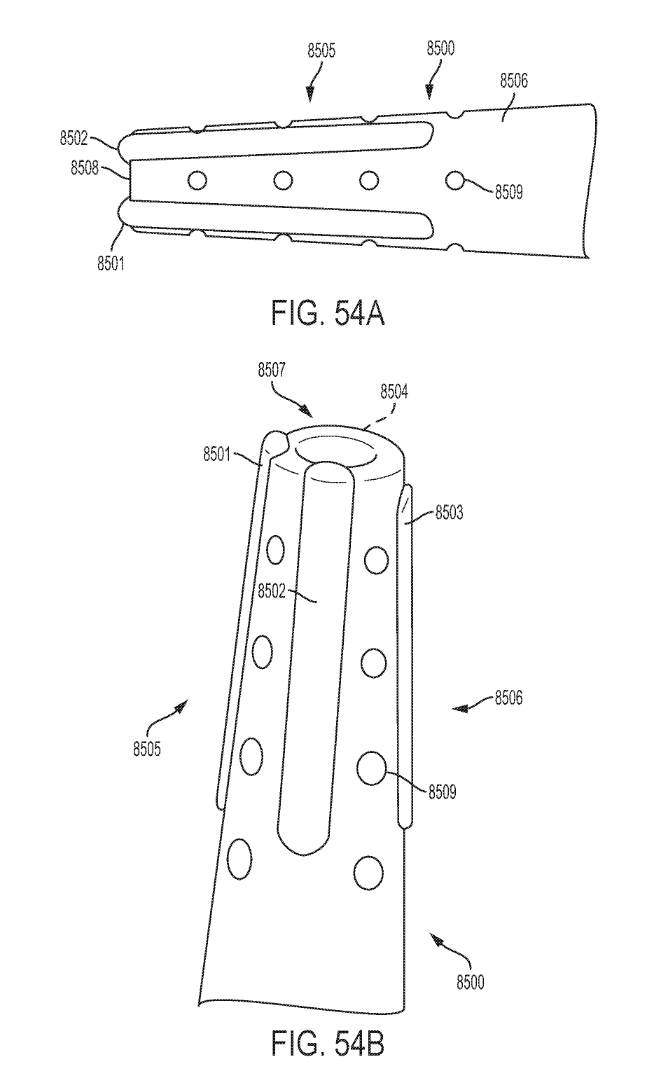

Now referring to FIGS. 54A,B, another embodiment of the end effector 8500 is illustrated here. The end effector 8500 has 4 in-line electrodes 8501-8504 provided at the distal end 8505 and on the side surface 8506 of the end effector 8500. The electrodes 8501-8504 may have a longitudinal configuration and extend along a longitudinal direction of the end effector 8500. The 4 electrodes 8501-8504 may include 2 pairs of electrodes of opposite polarities. The 4 electrodes 8501-8504 may be provided at the end effector 8500 such that each electrode is next to an electrode of the opposite polarity, as shown in the figure. The 2 positive electrodes 8501 and 8503 and the 2 negative electrodes 8502 and 8504 may be provided at the end effector 8500 alternatively. Of the 4 electrodes 8501-8504, 1 positive electrode 8501 and 1 negative electrode 8502 may extend to the tip 8507 of the end effector 8500 to provide coagulation at the tip 8507. The other positive electrode 8503 and the other negative electrode 8504 do not extend to the tip 8507 of the end effector 8500 and therefore, do not provide coagulation at the tip 8507. Therefore, the end effector 8500 may provide coagulation both at the tip 8507 and on the side surface 8506. The irrigation lumen 8509 may be provided on the side surface 8506, and the suction lumen 8508 may be provided at the tip 8507 of the end effector 8500.

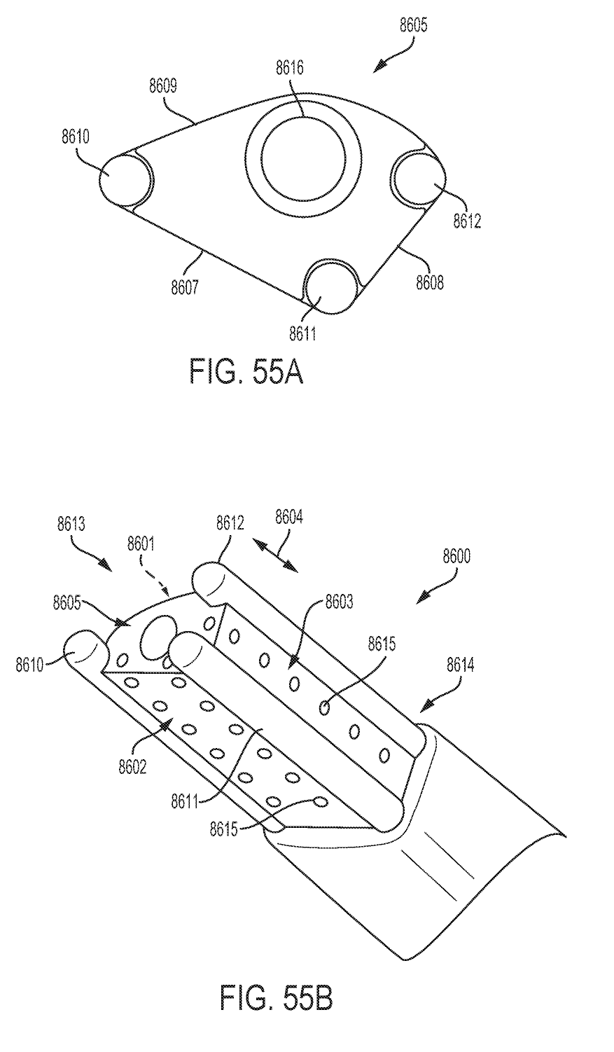

FIGS. 55A,B illustrate another example embodiment of the end effector 8600. As shown here, the end effector 8600 has three side surfaces 8601-8603 with the same length in a longitudinal direction 8604 of the end effector 8600, but different widths and surface configuration. The end effector 8600 may have two flat surfaces 8602 and 8603 and a curved surface 8601. One flat surface 8602 has a larger width than the other 8603. As shown in FIG. 55A, the distal face 8605 of the end effector 8600 has a long edge 8607, a short edge 8608, and a curved edge 8609. The electrodes 8610-8612 extend along the intersections of the three side surfaces 8601-8603 from the tip 8613 of the end effector 8600 to the proximal end 8614 of the end effector 8600. One electrode 8611 is of one polarity (e.g., positive or negative), and the other two 8610 and 8612 are of the opposite polarity (e.g., negative or positive, depending on the electrode 8611). The end effector 8600 can provide larger coagulation at the wider side surface 8602 and longer edge 8607 at the tip 8613 and smaller coagulation at the narrower side surface 8603 and the shorter edge 8608 at the tip 8613. The irrigation lumen 8615 may be provided on the side surfaces 8601-8603. The irrigation lumen 8615 may also be provided on the distal face 8605. The suction lumen 8616 may be provided on the distal face 8605.

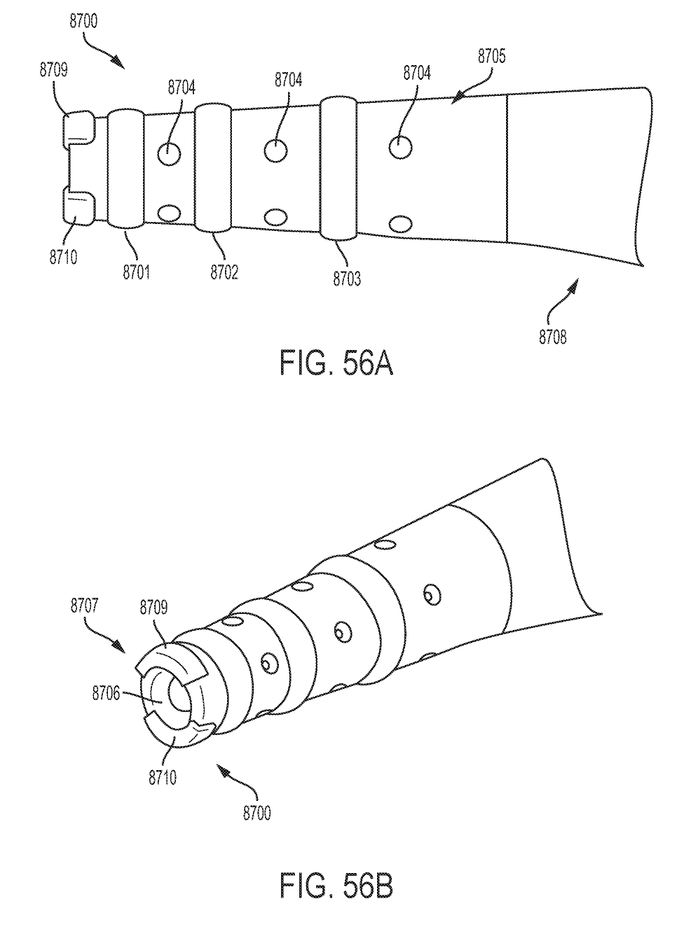

Now referring to FIGS. 56A,B, illustrated is an embodiment of the end effector 8700. The end effector 8700 also has circular electrodes 8701-8703. Irrigation fluid outlets 8704 are provided on the side surfaces 8705 between the electrodes 8701-8703 and between the electrode 8703 and the proximal end 8708 of the end effector 8700. Every two of the electrodes 8701-8703 next to each other may have the same or opposite polarities. For example, the electrode 8701 may be a positive electrode, and the electrodes 8702 and 8703 may be negative. The distance between the electrodes 8701 and 8702 may be smaller than the distance between the electrodes 8702 and 8703, so that the end effector 8700 can provide coagulation for areas of different sizes depending on the needs. The suction lumen 8706 is provided on the distal face 8707 of the end effector 8700. The two tips 8709 and 8710 at the distal face 8707 at the edge of the suction lumen 8706 may be conducting and be of two opposite polarities, so that coagulation can also be provided at the distal face 8707 of the end effector 8700.

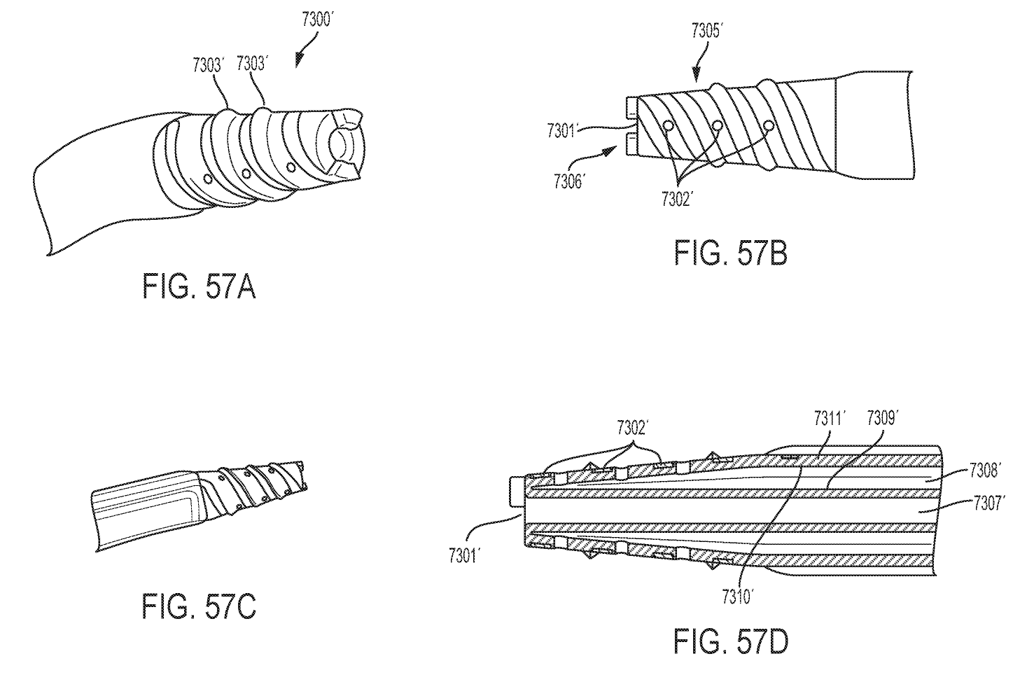

FIGS. 57A-D are illustrations of the spiral end effector. FIGS. 57A-D illustrate the end effector 7300′ from different angles. As shown here, the irrigation lumen 7302′ may be provided on the side surface 7305′ of the end effector 7300′. The suction lumen 7301′ may be provided at the distal face 7306′ of the end effector 7300′. Flow directing ridges or ribs 7303′ may be provided on the side surface to guide the flow of the irrigation fluid exiting the irrigation lumen 7302. FIG. 57C illustrates the electrodes extend from the end effector 7300′ into the elongate member that connects the end effector 7300′ to a handle of a surgical device. The handle and the surgical device are described above, but not shown here. FIG. 57D shows a cross-section of the end effector 7300′. As shown here, a suction tube 7307′ with the suction lumen 7301′ may be provided in the interior of the end effector 7300′. The interior space 7308′ of the end effector 7300′ between the outer surface 7309′ of the suction tube 7307′ and the inner surface 7310′ of the housing 7311′ of the end effector 7300′ can be used as the irrigation “tube”7308′ that surrounds the suction tube 7307′ and provides “all around” irrigation. The suction tube 7307′ and the suction lumen 7301′ may be manufactured separately from the other parts of the end effector 7300′ and then assembled into the end effector 7300′.

FIGS. 58 and 59A-F illustrate various views of yet another embodiment of the end effector 6200. As shown in these figures, the center fluid lumen may be provided on a molded non-conductive elastomeric (e.g. silicone) or plastic (e.g. high temperature polymers, ceramic injection molded, etc.) manifold component 6250 between the two probes 6202 and 6203. The manifold component 6250 may be compliant. One or more irrigation outlets 6204 and 6208 may be provided on a top surface 6205 and/or a distal surface 6206 of the component 6250. One or more suction outlets 6207 may be also provided on the distal surface 6206 of the component 6250 and below the two probes 6202 and 6203. Only one irrigation outlet 6208 and one suction outlet 6206 are shown in these figures. The one or more irrigation outlets 6204 and the one or more suction outlets 6207 may be formed from a single molded component 6250. The lateral irrigation outlets 6204 and the distal irrigation outlet 6208 are above the suction outlet 6207 because of the gravity of the fluid in most use cases. This embodiment provides a compact design of the end effector.

FIGS. 60 and 61A-D illustrate different views of an end effector 6400 with a triangular geometry and three electrodes 6301-6303. The electrodes 6301-6303 may be configured such that the electrode (one of 6301, 6302, and 6303) positioned on one of the edges of the triangle 6304 is one pole and the other two electrodes (the other two of 6301, 6302, and 6303) are the other pole of the bipolar electrosurgical end effector 6400. The electrodes 6301-6303 may be formed by plating a polymer (e.g. high temperature thermoplastic) with metal (e.g. nickel, gold, etc.) or by bonding or insert molding wire/sheet forms. As shown in these figures, the irrigation outlets 6305 are all on the same side of the end effector 6400, and suction is through a central lumen 6306 on the distal face 6307. The electrodes 6301-6303 are shown here to extend past the distal face 6307 of the suction lumen 6306 termination slightly. The electrodes 6301-6303 may pass the distal face 6307 as much as about 5 mm, but preferably less than about 2 mm.

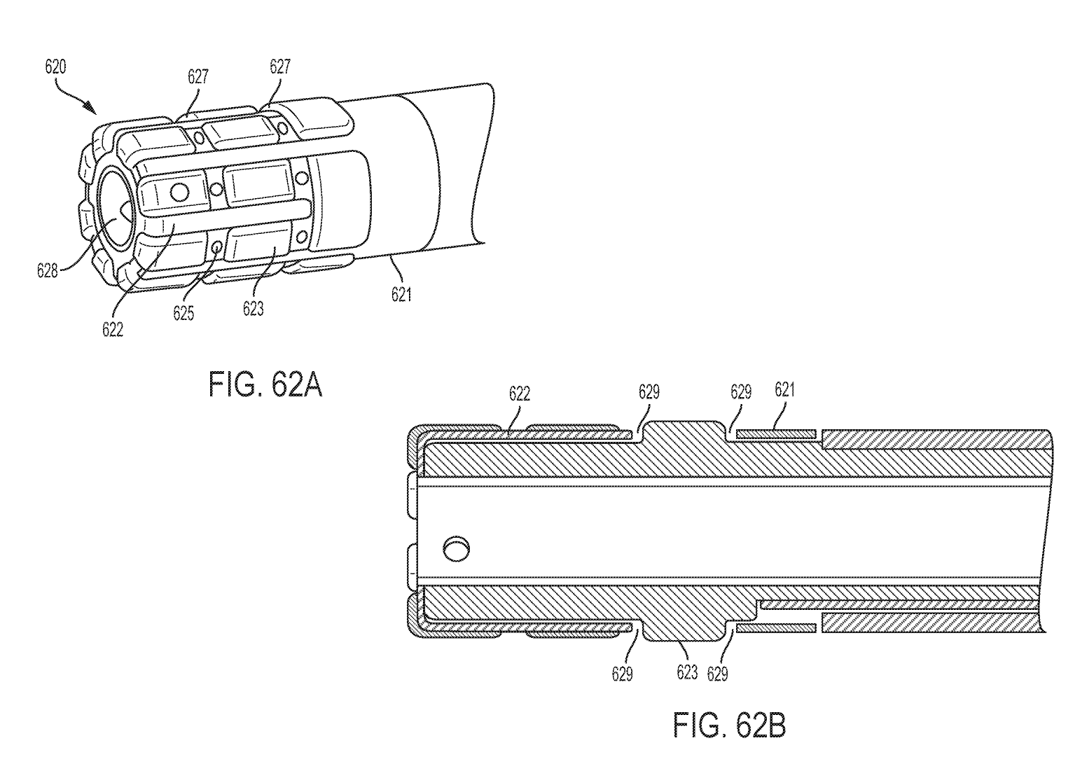

FIGS. 62A depicts another aspect of an end effector 620. It may be observed that end effector 620 is similar to that of end effector 1506a as depicted in FIG. 15B. End effector 620 includes an end effector body 623 having an outer surface and electrodes 621 and 622. Electrodes 621 and 622 are interdigitated electrodes having legs dispose parallel to a longitudinal axis of the end effector body 623. The end effector 620 also includes a fluid aspiration port 628 disposed on a distal face of the end effector body 623. End effector 620 also includes multiple fluid discharge ports 625 configured to deliver a fluid to the surface of the end effector body 623. In some aspects, the end effector body 623 may include one or more fluid channels 627 fabricated in the end effector body surface to direct a flow of the fluid from the fluid discharge ports 625, for example to permit the fluid to contact the electrodes 621 and 622.

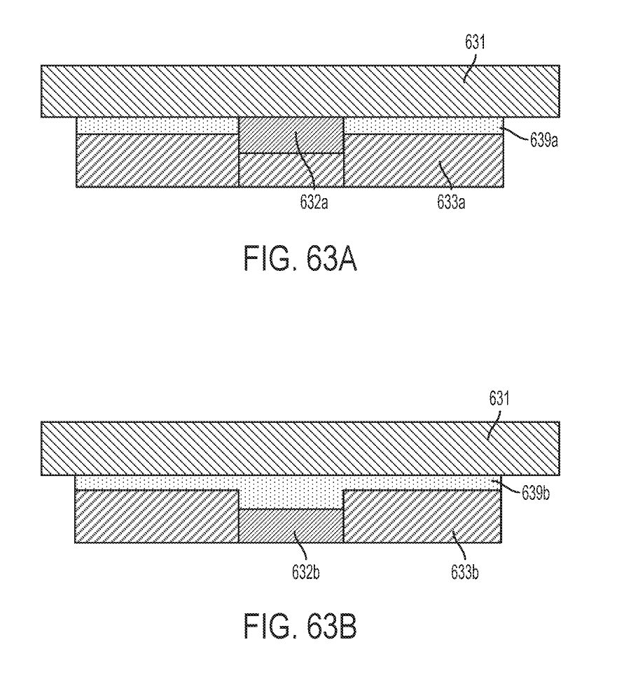

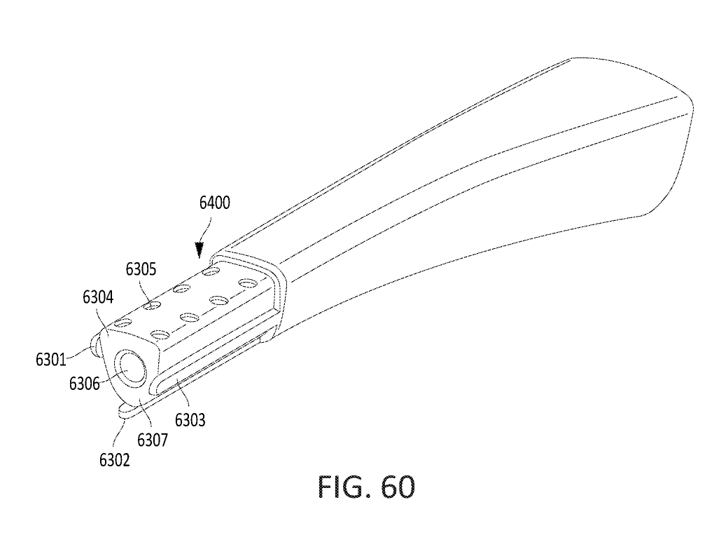

FIG. 62B depicts a cross sectional view of end effector 620. It may be particularly observed that each of electrode 621 and electrode 622 is not disposed on an exterior surface of the end effector body 623. Rather, electrode 621 and 622 is disposed within one or more recesses 629 fabricated in the surface of the end effector body. FIGS. 63A,B compare one aspect that may be associated with electrodes 621 and 622 as disposed within the recesses 629 with an end effector having electrodes disposed on an exterior surface of the end effector.

FIG. 63A depicts an end effector body 633a having an electrode 632a that possesses a surface that protrudes above the surface of the end effector body 633a. It may be observed that the application of the end effector body 633a to a tissue 631 may result in a direct contact of a surface of the electrode 632a to the tissue 631. Such direct contact may result in burning of the tissue 631 because a flow of fluid 639a on the surface of the end effector body 633a does not properly coat and/or cover the surface of the electrode 632a.

FIG. 63B depicts an end effector body 633b having an electrode 632b that is disposed within a recess in the end effector body 633b, similar to the configuration depicted in FIGS. 62A,B. It may be observed that the application of the end effector body 633b to a tissue 631 may not result in a direct contact of a surface of the electrode 632b to the tissue 631. Instead, the fluid flow 639b coats the surface of the electrode 632b preventing direct contact with the tissue 631, thereby reducing the possibility of tissue burns.

Several examples of end effectors for an electrosurgical device are depicted and disclosed herein. In some aspects, such electrosurgical devices may include end effectors having one or more electrodes that may extend from an interior portion of the end effector. Examples of such end effectors are depicted above in FIGS. 1-12. Alternative end effectors are depicted in FIGS. 13-63. Such alternative end effectors may be generally characterized as having an end effector body on which the electrodes are disposed. In some examples, the electrodes of such end effectors may be disposed on an exterior surface of the end effector bodies. In other examples, the electrodes may be disposed on one or more protuberance that form extensions of the exterior surface of the end effector bodies. In other examples, the electrodes may be disposed in one or more recesses formed in the exterior surface of the end effector bodies. The electrodes may include, without limitation, linear electrodes, interdigitated electrodes, or helical electrodes, as examples. Such examples of electrode shapes and/or dispositions are not limited to the examples depicted and/or disclosed herein.

Additionally, such end effectors may include one or more ports configured to discharge a fluid onto a surface of the end effector body. The end effectors may also include one or more channels configured to direct a flow the fluid on a surface of the end effector body. Alternatively, the end effectors may include one or more protuberances such as ridges that may also be configured to direct a flow the fluid on the surface of the end effector body. The geometries of such channels or such protuberances are not limited to the geometries depicted and disclosed herein. Such end effectors may also include one or more ports configured to aspirate a fluid external to the end effector and conduct the aspirated fluid to a retention reservoir, for example by means of a vacuum system. The one or more discharge ports and/or one or more aspiration ports may be disposed on any appropriate surface of the end effector body.

It may be understood that any of such end effectors as depicted in FIGS. 13-63 or similarly designed end effectors may be modular end effectors configured to be releasably attached to a modular electrosurgical device. Alternatively, any of such end effectors as depicted in FIGS. 13-63 may be fabricated as part of an electrosurgical device and may be configured to remain affixed to the electrosurgical device during normal use.

It will be appreciated that the terms “proximal” and “distal” are used throughout the specification with reference to a clinician manipulating one end of an instrument used to treat a patient. The term “proximal” refers to the portion of the instrument closest to the clinician and the term “distal” refers to the portion located furthest from the clinician. It will further be appreciated that for conciseness and clarity, spatial terms such as “vertical,” “horizontal,” “up,” or “down” may be used herein with respect to the illustrated embodiments. However, surgical instruments may be used in many orientations and positions, and these terms are not intended to be limiting or absolute.

Various aspects of surgical instruments are described herein. It will be understood by those skilled in the art that the various aspects described herein may be used with the described surgical instruments. The descriptions are provided for example only, and those skilled in the art will understand that the disclosed examples are not limited to only the devices disclosed herein, but may be used with any compatible surgical instrument or robotic surgical system.

Reference throughout the specification to “various aspects,” “some aspects,” “one example,” or “one aspect” means that a particular feature, structure, or characteristic described in connection with the aspect is included in at least one example. Thus, appearances of the phrases “in various aspects,” “in some aspects,” “in one example,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

While various aspects herein have been illustrated by description of several aspects and while the illustrative embodiments have been described in considerable detail, it is not the intention of the applicant to restrict or in any way limit the scope of the appended claims to such detail. Additional advantages and modifications may readily appear to those skilled in the art. For example, it is generally accepted that endoscopic procedures are more common than laparoscopic procedures. Accordingly, the present invention has been discussed in terms of endoscopic procedures and apparatus. However, use herein of terms such as “endoscopic”, should not be construed to limit the present invention to an instrument for use only in conjunction with an endoscopic tube (e.g., trocar). On the contrary, it is believed that the present invention may find use in any procedure where access is limited to a small incision, including but not limited to laparoscopic procedures, as well as open procedures.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

While several aspects have been described, it should be apparent, however, that various modifications, alterations and adaptations to those embodiments may occur to persons skilled in the art with the attainment of some or all of the advantages of the disclosure. For example, according to various aspects, a single component may be replaced by multiple components, and multiple components may be replaced by a single component, to perform a given function or functions. This application is therefore intended to cover all such modifications, alterations and adaptations without departing from the scope and spirit of the disclosure as defined by the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Various aspects of the subject matter described herein are set out in the following numbered examples:

### EXAMPLE 1

An end effector of an electrosurgical device, the end effector comprising:

an end effector body having a longitudinal body axis;

at least one distal fluid discharge port disposed in the end effector body;

at least one distal fluid aspiration port disposed in the end effector body;

a first electrode disposed on a first portion of a surface of the end effector body; and

a second electrode disposed on a second portion of the surface of the end effector body,

wherein the at least one distal fluid discharge port is configured to discharge a fluid therefrom, and

wherein the end effector body is configured to direct the discharged fluid to contact a surface of the first electrode and a surface of the second electrode.

### EXAMPLE 2

The end effector of example 1, wherein the first electrode comprises at least one first electrode component disposed parallel to the longitudinal body axis and the second electrode comprises at least one second electrode component disposed parallel to the longitudinal body axis.

### EXAMPLE 3

The end effector of any one or more of example 1 through example 2, wherein the first electrode comprises at least one first electrode component disposed helically along the longitudinal body axis and the second electrode comprises at least one second electrode component disposed helically along the longitudinal body axis.

### EXAMPLE 4

The end effector of any one or more of example 1 through example 3, wherein the first electrode comprises at least one circular first electrode component disposed orthogonal to the longitudinal body axis and the second electrode comprises at least one circular second electrode component disposed orthogonal to the longitudinal body axis.

### EXAMPLE 5

The end effector of any one or more of example 1 through example 4, wherein the end effector body comprises one or more body features.

### EXAMPLE 6

The end effector of example 5, wherein the one or more body features are configured to direct a flow of the discharged fluid about the surface of the end effector body.

### EXAMPLE 7

The end effector of any one or more of example 5 through example 6, wherein the body features comprise one or more protruding features from the surface of the end effector body.

### EXAMPLE 8

The end effector of example 7, wherein the one or more protruding features comprise one more protruding features helically disposed about the end effector body and oriented along the longitudinal body axis.

### EXAMPLE 9

The end effector of example 7, wherein the one or more protruding features comprise one or more raised rings from the surface of the end effector body.

### EXAMPLE 10

The end effector of any one or more of example 7 through example 9, wherein at least a portion of the first electrode is disposed on a surface of the one or more protruding features.

### EXAMPLE 11

The end effector of any one or more of example 7 through example 10, wherein at least a portion of the second electrode is disposed on a surface of the one or more protruding features.

### EXAMPLE 12

The end effector of example 5, wherein the body features comprise one or more channels in the surface of the end effector body.

### EXAMPLE 13

The end effector of example 12, wherein the one or more channels comprise one more channels helically disposed within the surface of the end effector body and oriented along the longitudinal body axis.

### EXAMPLE 14

The end effector of example 12, wherein the one or more channels comprise one or more channels disposed parallel to the longitudinal body axis of the end effector body.

### EXAMPLE 15

The end effector of any one or more of example 12 through example 14, wherein at least a portion of the first electrode is disposed within the one or more channels.

### EXAMPLE 16

The end effector of any one or more of example 12 through example 15, wherein at least a portion of the second electrode is disposed within the one or more channels.

### EXAMPLE 17

The end effector of any one or more of example 1 through example 16, wherein the at least one distal fluid aspiration port is disposed at a distal end of the end effector body.

### EXAMPLE 18

The end effector of any one or more of example 1 through example 16, wherein at least a portion of the end effector body is tapered towards a distal end of the end effector body.

### EXAMPLE 19

An electrosurgical device comprising:

an end effector comprising:
an end effector body;
a first electrode disposed on a first portion of a surface of the end effector body;
a second electrode disposed on a second portion of the surface of the end effector body;
at least one fluid discharge port disposed on a surface of the end effector body; and
at least one fluid aspiration port disposed at a distal end of the end effector body;

a shaft having a longitudinal shaft axis, wherein a distal shaft end is in mechanical communication with a proximal end of the end effector body; and

a housing having a longitudinal housing axis, the housing comprising:
a fluid source port configured to receive a first fluid from a first fluid source and fluidically coupled to the at least one distal fluid discharge port;
a fluid evacuation port configured to deliver a second fluid to a vacuum source and fluidically coupled to the at least one distal fluid aspiration port;
a first fluid control fluidically coupled to the at least one fluid discharge port; and
a combination control, configured to regulate a flow of the first fluid to the first fluid control and to regulate an amount of power delivered to the first electrode and the second electrode from a power source.

### EXAMPLE 20

The electrosurgical device of example 19, wherein a distal end of the housing is in mechanical communication with a proximal end of the shaft end configured so that the longitudinal housing axis forms an acute angle with respect to the longitudinal shaft axis.

### EXAMPLE 21

The electrosurgical device of any one or more of example 19 through example 20 further comprising an end effector unit comprising the end effector body and an end effector body extension mechanically coupled to a proximal portion of the end effector body.

### EXAMPLE 22

The electrosurgical device of example 21 wherein the end effector unit is disposed within an interior space of the shaft and at least a portion of an evacuation tube is disposed within an interior space of the end effector unit.

### EXAMPLE 23

The electrosurgical device of example 22, wherein the end effector unit comprises one or more fluid vents disposed proximate to an outer surface of the at least portion of the evacuation tube thereby creating a fluid space defined by the outer surface of the evacuation tube and an inner surface of the end effector body extension.

### EXAMPLE 24

The electrosurgical device of example 23, wherein the fluid space is fluidically coupled to the at least one fluid discharge port.

### EXAMPLE 25

The electrosurgical device of any one or more of example 22 through example 24, wherein the evacuation tube is fludically coupled to the fluid evacuation port.

### EXAMPLE 26

The electrosurgical device of any one or more of example 22 through example 25, wherein the evacuation tube is electrically conducting and the first electrode is electrically coupled to the evacuation tube.

### EXAMPLE 27

The electrosurgical device of any one or more of example 19 through example 26 wherein the end effector body comprises one or more channels configured to receive the first electrode and the second electrode.

### EXAMPLE 28

An electrosurgical device comprising:

an end effector comprising:
an end effector body;
a first electrode disposed on a first portion of a surface of the end effector body;
a second electrode disposed on a second portion of the surface of the end effector body;
at least one fluid discharge port disposed at a distal end of the end effector body; and
at least one fluid aspiration port disposed at the distal end of the end effector body;

a shaft having a longitudinal shaft axis, wherein a distal shaft end is in mechanical communication with a proximal end of the end effector body, and wherein the shaft is configured to assume a bent configuration upon receiving an application of a first force orthogonal to a longitudinal axis of the shaft; and

a housing comprising:
a fluid source port configured to receive a first fluid from a first fluid source and fluidically coupled to the at least one distal fluid discharge port; and
a fluid evacuation port configured to deliver a second fluid to a vacuum source and fluidically coupled to the at least one distal fluid aspiration port.

### EXAMPLE 29

The electrosurgical device of example 28, wherein the shaft is configured to remain in the bent configuration after the removal of the first force applied to the shaft.

### EXAMPLE 30

The electrosurgical device of any one or more of example 28 through example 29, wherein the shaft is configured to assume an unbent configuration and upon receiving an application of a second force to the shaft, wherein the second force is an opposing force to the first force.

### EXAMPLE 31

The electrosurgical device of any one or more of example 28 through example 30, wherein the first electrode disposed on the first portion of the surface of the end effector body is helically wound about a longitudinal axis of the end effector body, and

wherein the second electrode disposed on the second portion of the surface of the end effector body is helically wound about the longitudinal axis of the end effector body.

### EXAMPLE 32

The electrosurgical device of any one or more of example 28 through example 31, wherein the first electrode disposed on the first portion of the surface of the end effector body comprises a first plurality of legs disposed on the surface of the end effector body and parallel to a longitudinal axis of the end effector body, and

wherein the second electrode disposed on the second portion of the surface of the end effector body comprises a second plurality of legs disposed on the surface of the end effector body and parallel to the longitudinal axis of the end effector body.

What is claimed is:

1. An end effector of an electrosurgical device, the end effector comprising:
an end effector body having a longitudinal body axis;
at least one distal fluid discharge port disposed in the end effector body;
at least one distal fluid aspiration port disposed in the end effector body;
a first electrode disposed on a first portion of a surface of the end effector body; and
a second electrode disposed on a second portion of the surface of the end effector body,
wherein the at least one distal fluid discharge port is configured to discharge a fluid therefrom, and
wherein the end effector body is configured to direct the discharged fluid to contact a surface of the first electrode and a surface of the second electrode.

2. The end effector of claim 1, wherein the first electrode comprises at least one first electrode component disposed parallel to the longitudinal body axis and the second electrode comprises at least one second electrode component disposed parallel to the longitudinal body axis.

3. The end effector of claim 1, wherein the first electrode comprises at least one first electrode component disposed helically along the longitudinal body axis and the second electrode comprises at least one second electrode component disposed helically along the longitudinal body axis.

4. The end effector of claim 1, wherein the first electrode comprises at least one circular first electrode component disposed orthogonal to the longitudinal body axis and the second electrode comprises at least one circular second electrode component disposed orthogonal to the longitudinal body axis.

5. The end effector of claim 1, wherein the end effector body comprises one or more body features.

6. The end effector of claim 5, wherein the one or more body features are configured to direct a flow of the discharged fluid about the surface of the end effector body.

7. The end effector of claim 5, wherein the body features comprise one or more protruding features from the surface of the end effector body.

8. The end effector of claim 7, wherein the one or more protruding features comprise one more protruding features helically disposed about the end effector body and oriented along the longitudinal body axis.

9. The end effector of claim 7, wherein the one or more protruding features comprise one or more raised rings from the surface of the end effector body.

10. The end effector of claim 7, wherein at least a portion of the first electrode is disposed on a surface of the one or more protruding features.

11. The end effector of claim 7, wherein at least a portion of the second electrode is disposed on a surface of the one or more protruding features.

12. The end effector of claim 5, wherein the body features comprise one or more channels in the surface of the end effector body.

13. The end effector of claim 12, wherein the one or more channels comprise one more channels helically disposed within the surface of the end effector body and oriented along the longitudinal body axis.

14. The end effector of claim 12, wherein the one or more channels comprise one or more channels disposed parallel to the longitudinal body axis of the end effector body.

15. The end effector of claim 12, wherein at least a portion of the first electrode is disposed within the one or more channels.

16. The end effector of claim 12, wherein at least a portion of the second electrode is disposed within the one or more channels.

17. The end effector of claim 1, wherein the at least one distal fluid aspiration port is disposed at a distal end of the end effector body.

18. The end effector of claim 1, wherein at least a portion of the end effector body is tapered towards a distal end of the end effector body.

19. An electrosurgical device comprising:
an end effector comprising:
an end effector body;
a first electrode disposed on a first portion of a surface of the end effector body;
a second electrode disposed on a second portion of the surface of the end effector body;
at least one fluid discharge port disposed on a surface of the end effector body; and
at least one fluid aspiration port disposed at a distal end of the end effector body;
a shaft having a longitudinal shaft axis, wherein a distal shaft end is in mechanical communication with a proximal end of the end effector body; and
a housing having a longitudinal housing axis, the housing comprising:
a fluid source port configured to receive a first fluid from a first fluid source and fluidically coupled to the at least one distal fluid discharge port;
a fluid evacuation port configured to deliver a second fluid to a vacuum source and fluidically coupled to the at least one distal fluid aspiration port;
a first fluid control fluidically coupled to the at least one fluid discharge port; and
a combination control, configured to regulate a flow of the first fluid to the first fluid control and to regulate an amount of power delivered to the first electrode and the second electrode from a power source.

20. The electrosurgical device of claim 19, wherein a distal end of the housing is in mechanical communication with a proximal end of the shaft end configured so that the longitudinal housing axis forms an acute angle with respect to the longitudinal shaft axis.

21. The electrosurgical device of claim 19 further comprising an end effector unit comprising the end effector body and an end effector body extension mechanically coupled to a proximal portion of the end effector body.

22. The electrosurgical device of claim 21 wherein the end effector unit is disposed within an interior space of the shaft and at least a portion of an evacuation tube is disposed within an interior space of the end effector unit.

23. The electrosurgical device of claim 22, wherein the end effector unit comprises one or more fluid vents disposed proximate to an outer surface of the at least portion of the evacuation tube thereby creating a fluid space defined by the outer surface of the evacuation tube and an inner surface of the end effector body extension.

24. The electrosurgical device of claim 23, wherein the fluid space is fluidically coupled to the at least one fluid discharge port.

25. The electrosurgical device of claim 22, wherein the evacuation tube is fludically coupled to the fluid evacuation port.

26. The electrosurgical device of claim 22, wherein the evacuation tube is electrically conducting and the first electrode is electrically coupled to the evacuation tube.

27. The electrosurgical device of claim 19 wherein the end effector body comprises one or more channels configured to receive the first electrode and the second electrode.

28. An electrosurgical device comprising:
an end effector comprising:
an end effector body;
a first electrode disposed on a first portion of a surface of the end effector body;
a second electrode disposed on a second portion of the surface of the end effector body;
at least one fluid discharge port disposed at a distal end of the end effector body; and
at least one fluid aspiration port disposed at the distal end of the end effector body;
a shaft having a longitudinal shaft axis, wherein a distal shaft end is in mechanical communication with a proximal end of the end effector body, and wherein the shaft is configured to assume a bent configuration upon receiving an application of a first force orthogonal to a longitudinal axis of the shaft; and
a housing comprising:
a fluid source port configured to receive a first fluid from a first fluid source and fluidically coupled to the at least one distal fluid discharge port; and
a fluid evacuation port configured to deliver a second fluid to a vacuum source and fluidically coupled to the at least one distal fluid aspiration port.

29. The electrosurgical device of claim 28, wherein the shaft is configured to remain in the bent configuration after the removal of the first force applied to the shaft.

30. The electrosurgical device of claim 28, wherein the shaft is configured to assume an unbent configuration and upon receiving an application of a second force to the shaft, wherein the second force is an opposing force to the first force.

31. The electrosurgical device of claim 28, wherein the first electrode disposed on the first portion of the surface of the end effector body is helically wound about a longitudinal axis of the end effector body, and wherein the second electrode disposed on the second portion of the surface of the end effector body is helically wound about the longitudinal axis of the end effector body.

32. The electrosurgical device of claim 28, wherein the first electrode disposed on the first portion of the surface of the end effector body comprises a first plurality of legs disposed on the surface of the end effector body and parallel to a longitudinal axis of the end effector body, and
wherein the second electrode disposed on the second portion of the surface of the end effector body comprises a second plurality of legs disposed on the surface of the end effector body and parallel to the longitudinal axis of the end effector body.
